(19) United States
(12) Patent Application Publication
(10) Pub. No.: US 2026/0250351 A1
(43) Pub. Date: Aug. 27, 2026

(54) TRANSCRIPTIONAL THERAPY BASED-LIPID NANOPARTICLES AND MRNA FOR THE TREATMENT OF END-STAGE LIVER DISEASE

(71) Applicants: University of Pittsburgh - Of the Commonwealth System of Higher Education, Pittsburgh, PA (US); The Trustees of the University of Pennsylvania, Philadelphia, PA (US)

(72) Inventors: Alejandro Soto-Gutierrez, Pittsburgh, PA (US); Aaron W. Bell, West Mifflin, PA (US); Alina Krystyna Ostrowska, McKees Rocks, PA (US); Edgar Naoe Tafaleng, Pittsburgh, PA (US); Ira Jacob Fox, Pittsburgh, PA (US); Lanuza Alaby Pinheiro Faccioli, Pittsburgh, PA (US); Nils Haep, Berlin (DE); Ricardo Diaz-Aragon, Pittsburgh, PA (US); Rodrigo Machado Florentino, Pittsburgh, PA (US); Takashi Motomura, Pittsburgh, PA (US); Drew Weissman, Philadelphia, PA (US); Mohamad-Gabriel Alameh, Philadelphia, PA (US)

(73) Assignees: University of Pittsburgh - Of the Commonwealth System of Higher Education, Pittsburgh, PA (US); The Trustees of the University of Pennsylvania, Philadelphia, PA (US)

(21) Appl. No.: 18/726,767

(22) PCT Filed: Jan. 6, 2023
(86) PCT No.: PCT/US2023/060211

§ 371 Date: Jul. 3, 2024

Related U.S. Application Data
(60) Provisional application No. 63/297,188, filed on Jan. 6, 2022.
(60) Provisional application No. 63/420,464, filed on Oct. 28, 2022.

(51) Int. Cl. C07K 14/705; A61K 38/17; A61K 48/00; A61P 1/16; C12N 15/88

(52) U.S. Cl. CPC C07K 14/70567; A61K 38/1783; A61K 48/0033; A61K 48/005; A61P 1/16; C12N 15/88

(57) ABSTRACT

Methods are disclosed for treating end-stage liver failure in a subject that include administering to the subject a therapeutically effective amount of a recombinant mRNA encoding HNF4α isoform 2. The methods can include administering a lipid nanoparticle including the recombinant mRNA to the subject. Also disclosed are recombinant mRNA encoding an HNF4α isoform 1 or HNF4α isoform 2, and lipid nanoparticles include the recombinant mRNA. Methods are also for treating liver disease or end-stage liver failure in a subject that include administering to the subject a therapeutically effective amount of a recombinant mRNA encoding HNF4α isoform 1.

### CROSS REFERENCE TO RELATED APPLICATIONS

This application claims the benefit under 35 U.S.C. § 119(e) and the Paris Convention of U.S. Provisional Application No. 63/297,188, filed Jan. 6, 2022; and U.S. Provisional Application No. 63/420,464, filed Oct. 28, 2022, each of which are incorporated herein by reference in their entireties.

### FIELD OF THE DISCLOSURE

This relates to the treatment of liver failure, specifically to the use of mRNA encoding hepatocyte nuclear factor 4 alpha (HNF4α) isoform 1 (HNF4α1) and isoform 2 (HNF4α2) to treat end-stage liver disease.

### REFERENCE TO AN ELECTRONIC SEQUENCE LISTING

The contents of the electronic sequence listing (8123-107342-03 SL.xml; Size: 52,944 bytes; and Date of Creation: Jan. 5, 2023) is herein incorporated by reference in its entirety.

### BACKGROUND

Chronic liver injury results in cirrhosis and end-stage liver disease (ESLD) which represents a leading cause of death worldwide (roughly 1 million deaths annually), affecting people in their most productive years of life. Medical therapy such as optimizing fluid and electrolyte balance, portal hypertension control interventions, treatment for coagulopathy and measures to control hepatic encephalopathy can extend life, but the only definitive treatment is liver transplantation (LT). However, LT remains limited by access to quality donor organs and 75% of patients having complications in the long-term (5-20 years post-transplant) such as infection, malignancy, and renal insufficiency. The degeneration of healthy-functioning liver to cirrhosis and ESLD involves a dynamic process of hepatocyte damage, diminished hepatic function and altered hepatic matrix with portal hypertension. Thus, a therapy that can substitute for organ transplantation is highly desirable.

### SUMMARY

In some aspects, disclosed in a recombinant mRNA encoding an HNF4α1 or HNF4α2. In additional aspects, disclosed is a codon-optimized mRNA encoding HNF4α1 or HNF4α2. In more aspects, the recombinant mRNA comprises:
i) a nucleic acid sequence at least 95% identical to one of
   a) SEQ ID NO: 2 [HNF4α2],
   b) SEQ ID NO: 22 [HNFα1]; or
   c) SEQ ID NO: 30 [HNFα1];
   and
ii) optionally comprises one or more of:
   d) a 5′ capping structure,
   e) a promoter,
   f) a nucleic acid molecule encoding a signal peptide,
   g) a 5″ untranslated region (UTR),
   h) a 3′ UTR, and
   i) a polyA tail.

In some aspects, the recombinant mRNA incudes one of: a) a nucleic acid sequence at least 95% identical to SEQ ID NO: 2 or a nucleic acid sequence at least 95% identical to SEQ ID NO: 20. In further aspects, the recombinant mRNA optionally comprises one or more of: d) nucleic acid molecule encoding a signal peptide; e) a 5″ untranslated region (UTR), and f) a 3′ UTR. In one aspect, the equivalents of codon optimized polynucleotides retain one or more nucleotide alterations of the codon optimized sequence as compared to wild-type coding mRNA. Compositions and polynucleotides encoding the mRNA are further provided herein.

In further aspects, methods are disclosed for treating liver disease or end-stage liver failure in a subject. These methods include administering to the subject a therapeutically effective amount of a recombinant mRNA encoding HNF4α1, thereby treating the liver disease or end-stage liver failure in the subject

Methods are disclosed herein for treating end-stage liver failure in a subject. These methods include administering to the subject a therapeutically effective amount of a recombinant mRNA encoding HNF4α2, thereby treating the end-stage liver failure in the subject.

The foregoing and other features and advantages of the disclosed subject matter will become more apparent from the following detailed description of several aspects which proceeds with reference to the accompanying figures.

### BRIEF DESCRIPTION OF THE FIGURES

FIGS. 1A-1C. Evaluation of the lipid Nanoparticle (LNP) receptors, low density lipoprotein (LDLR) and liver related protein (LRP1), in normal and cirrhotic livers. (A) quantitative polymerase chain reaction (qPCR) for LDLR and LRP1 in normal isolated hepatocytes (n=7) and cirrhotic human hepatocytes classified as Child-Pugh B (n=7) and Child-Pugh C (=7) showing no difference at the mRNA levels of these receptors among the groups. (B) Immunostaining of LDLR and LRP1 in normal and cirrhotic livers. The diseased livers, classified as Child Pugh B and C, have a slight reduction in the expression of these proteins, but still expression in the hepatocyte. (C) A quantitative analysis of the protein level of LDLR and LRP1 in normal human isolate hepatocyte (n=9) and cirrhotic human hepatocytes classified as Child-Pugh B (n=8) and Child-Pugh C (=8). LDLR expression was decrease in both cirrhotic hepatocyte cohort in comparison with the normal hepatocytes (p=0.0116). A reduction of LRP1 expression was observed between normal hepatocytes and cirrhotic hepatocytes in the most advanced stage of the end-stage liver disease, Child-Pugh C (p<0.0001).

FIG. 2. LNP FM-1520A shows a high delivery efficiency in cirrhotic human hepatocytes. LNP FM-1520A was used to deliver green fluorescent protein (GFP) into primary human hepatocytes from a cirrhotic liver. Different doses of LPNs FM1020A (0.1 μg-4 μg) were evaluated over 72 hours under the presence and absence of FBS. The highest number of GFP positive hepatocytes could be observed at 48 and 72 hours (90%). The presence of 5% FBS showed a higher delivery efficiency (90%) compared to 0% and 10% FBS (65% and 80%). Immunofluorescence micrographs show representative images of the GFP kinetic when delivered by LNP FM-1520A (Igg).

FIG. 3. Evaluation of mRNA-HNF4a bioactivity in primary human Hepatocytes isolated from explanted cirrhotic liver with end-stage liver disease. Primary human hepatocytes were isolated from a cirrhotic liver from a 67 year-old female patient with non-alcoholic steatohepatitis (NASH) and End-Stage Liver Disease undergoing liver transplantation. Different concentrations of mRNA-HNF4α2 (0.1 μg-0.5 μg) were transfected into primary human hepatocytes from a cirrhotic liver using lipofectamine. The post-translational levels and the functionality of HNF4a were evaluated over 120 hour (h) by Western Blot. mRNA-HNF4α2 can restore HNF4a protein expression and function in cirrhotic human hepatocytes. The primary human hepatocytes from a cirrhotic liver were infected with 0.1 μg, 0.25 μg or 0.5 μg mRNA-HNF4α2. Post-translational HNF4a protein levels were evaluated by Western Blot at 6 h, 12, 24 h, 48h, 72h, 96 h and 120h. The highest HNF4a protein levels could be observed at 12 hours after transfection. HNF4a protein was detectable for 96 h after transfection with mRNA HNF4α. Without transfection of mRNA-HNF4a the HNF4a protein levels were undetectably low before (0h) and during our culture (6 h-120h) as compared to a healthy human hepatocyte.

FIG. 4. Schematic representation of the various steps involved in testing the effectiveness of a lipid nanoparticle including HNF4α2 mRNA (LNP-mRNA-HNF4α2) to reprogram hepatic gene circuits in hepatocytes isolated from the livers of patients who underwent liver transplantation for end-stage liver disease. Tissue specimens are taken from explanted livers of patients who underwent transplant for end-stage irreversibly decompensated liver failure due to NASH and alcohol-consumption. The tissue specimens are processed to obtain isolated primary end-stage cirrhotic hepatocytes which are then treated with LNP-mRNA-HNF4α2. Changes in the levels of HNF4α2, other liver-enriched transcription factors, and hepatocyte-expressed proteins are evaluated to determine whether LNP-mRNA-HNF4α2 treatment can reprogram hepatic gene circuits.

FIGS. 5A-5C. LNP-mRNA-HNF4α2 treatment of primary human hepatocytes isolated from the liver of a patient (HH113) who underwent liver transplantation for NASH results in increased HNF4α2 expression and subsequent upregulation of downstream hepatocyte-expressed proteins. (A) Western blot analysis of primary end-stage cirrhotic hepatocytes treated with increasing doses of LNP-mRNA-HNF4α2 shows increased HNF4α2 protein levels starting at 12 hours and with peak expression at 24 hours after treatment. (B) Fluorescence imaging of primary end-stage cirrhotic hepatocytes treated with increasing doses of LNP-mRNA-eGFP shows increased percentage of eGFP+ cells with maximal efficiency at a dose of 2 μg. (C) Western blot analysis shows that the levels of Albumin and UGT1A1 in primary end-stage cirrhotic hepatocytes treated with 2 μg of LNP-mRNA-HNF4α2 increases at 48 and 72 hours after treatment. In contrast, the levels of Albumin and UGT1A1 in control untreated cells decline from 48 to 72 hours after treatment.

FIGS. 6A-6C. LNP-mRNA-HNF4α2 treatment of primary human hepatocytes isolated from the liver of a second patient (HH114) who underwent liver transplantation for NASH results in increased HNF4α2 expression and subsequent upregulation of downstream hepatocyte-expressed proteins. (A) Western blot analysis of primary end-stage cirrhotic hepatocytes treated with increasing doses of LNP-mRNA-HNF4α2 shows increased HNF4α2 protein levels starting at 12 hours and with peak expression at 24 hours after treatment. (B) Fluorescence imaging of primary end-stage cirrhotic hepatocytes treated with increasing doses of LNP-mRNA-eGFP shows increased percentage of eGFP+ cells with maximal efficiency at a dose of 2 μg. (C) Western blot analysis shows that the levels of Albumin and UGT1A1 in primary end-stage cirrhotic hepatocytes treated with 2 μg of LNP-mRNA-HNF4α2 increases at 48 and 72 hours after treatment. In contrast, the levels of Albumin and UGT1A1 in control untreated cells decline from 48 to 72 hours after treatment.

FIGS. 7A-7C. LNP-mRNA-HNF4α2 treatment of primary human hepatocytes isolated from the liver of a third patient (HH120) who underwent liver transplantation for Alcoholic cirrhosis results in increased HNF4α2 expression and subsequent upregulation of downstream hepatocyte-expressed proteins. (A) Western blot analysis of primary end-stage cirrhotic hepatocytes treated with increasing doses of LNP-mRNA-HNF4α2 shows increased HNF4α2 protein levels starting at 12 hours and at 24 hours after treatment. (B) Fluorescence imaging of primary end-stage cirrhotic hepatocytes treated with increasing doses of LNP-mRNA-eGFP shows a high percentage of eGFP+ cells with 100% efficiency at a dose between 0.25 μg and 2 μg. (C) Western blot analysis shows that the levels of Albumin and UGT1A1 in primary end-stage cirrhotic hepatocytes treated with 2 ug of LNP-mRNA-HNF4α2 increases at 48 and 72 hours after treatment. In contrast, the levels of Albumin and UGT1A1 in control untreated cells decline from 48 to 72 hours after treatment.

FIGS. 8A-8B. LNP-mRNA-HNF4α2 treatment of primary human hepatocytes isolated from the liver of a patient (HH120) who underwent liver transplantation for alcoholic cirrhosis results in increased coagulation factor VII protein expression and an increase in the number of HNF4α2 positive cells. (A) Western blot analysis of primary end-stage cirrhotic hepatocytes treated with 2 ug LNP-mRNA-HNF4α2 show increased Coagulation factor VII (CFVII) protein levels starting at 12 hours and with peak expression at 24 hours after treatment. (B) Immunofluorescence staining of primary end-stage cirrhotic hepatocytes treated with 2 ug LNP-mRNA-HNF4α2 shows an increased percentage of HNF4α+ cells. About 25% of untreated primary cirrhotic human hepatocytes were positive for HNF4a and around 70% of the LNP-mRNA-HNF4α2 treated hepatocytes were positive for HNF4a 24 hours after treatment. 92.1% of the HNF4a positive hepatocytes showed nuclear and cytoplasmic localization and 7.9% showed both nuclear/cytoplasmic localization.

FIGS. 9A-9C. LNP-mRNA-HNF4α2 treatment of primary human hepatocytes isolated from the liver of a fourth patient (HH121) who underwent liver transplantation for Alcoholic cirrhosis results in increased HNF4α2 expression and subsequent upregulation of downstream hepatocyte-expressed proteins. (A) Western blot analysis of primary end-stage cirrhotic hepatocytes treated with increasing doses of LNP-mRNA-HNF4α2 shows increased HNF4α2 protein levels starting at 12 hours and with peak expression at 24 hours after treatment. (B) Fluorescence imaging of primary end-stage cirrhotic hepatocytes treated with increasing doses of LNP-mRNA-eGFP shows a high percentage of eGFP+ cells with 100% efficiency at a dose between 0.25 μg and 2 μg. (C) Western blot analysis shows that the levels of Albumin and UGT1A1 in primary end-stage cirrhotic hepatocytes treated with 2 ug of LNP-mRNA-HNF4α2 increases at 24, 48 and 72 hours after treatment. In contrast, the levels of Albumin and UGT1A1 in control untreated cells decline from 24, 48 to 72 hours after treatment.

FIGS. 10A-10B. LNP-mRNA-HNF4α2 treatment of primary human hepatocytes isolated from the liver of a patient (HH121) who underwent liver transplantation for Alcoholic cirrhosis results in increased Coagulation factor VII protein expression and an increase in the number of HNF4α2 positive cells. (A) Western blot analysis of primary end-stage cirrhotic hepatocytes treated with 2 g LNP-mRNA-HNF4α2 show increased coagulation factor VII (CFVII) protein levels with most different from the untreated control at 24 hours after treatment. (B) Immunofluorescence staining of primary end-stage cirrhotic hepatocytes treated with 2 μg LNP-mRNA-HNF4α2 shows an increased percentage of HNF4α+ cells. Around 15% of untreated primary cirrhotic human hepatocytes were positive for HNF4a and around 55% of the LNP-mRNA-HNF4α2 treated hepatocytes were positive for HNF4a 24 hours after treatment. 76.1% of the HNF4a positive hepatocytes showed nuclear and cytoplasmic localization and 23.9% showed both nuclear/cytoplasmic localization.

FIGS. 11A-11B. Evaluation of P1 and P2-HNF4a expression in primary human hepatocytes isolated from explanted liver with cirrhosis and end-stage liver disease. Only the P1-HNF4α2 isoform leads to an upregulation of downstream hepatocyte-expressed genes. A) Fluorescence imaging of freshly isolated human hepatocytes from explanted cirrhotic liver with end-stage liver disease who underwent liver transplantation. Isolated hepatocytes were transfected with plasmids (GFP, P1-HNF4α2 and P2-HNF4α7) using lipofectamine. Transfection efficiency was 49%. Only transfection with the P1-HNF4α2 not with the P2-HNF4α7 leads to increased expression of downstream mRNA expression of albumin and CFVII 48 hours after transfection. Statistical analysis was performed by Welch's T test. B) Western blot analysis for either P1-(isoform 2) or P2-(isoform 8) HNF4a of healthy control hepatocytes, cirrhotic human hepatocytes isolated from explanted livers of patients who underwent liver transplantation due to cirrhosis (NASH or Alcohol-induced cirrhosis) and End-Stage Liver Disease (Child Pugh B and C). As shown in previous figures, treatment with LNP-mRNA-HNF4α2 increases specifically the P1-HNF4a isoform expression not the P2-HNF4a isoform expression.

FIGS. 12A-12C. Evaluation of AAV-HNF4α2 transduction and system efficiency in primary human hepatocytes isolated from explanted cirrhotic livers with End-Stage Liver Disease. Primary human hepatocytes from human explanted cirrhotic livers with end-stage liver disease (Child Pugh “C”) (A) were transduced with two different adeno-associated viruses (AAV-10A2 and AAV-LKO3) at an MOI of 10\hat{ }5 and demonstrated that transduction efficiency was 65% for AAV-LK03 and 80% for AAV10A2 when using a GFP tag. (B) Changes in mRNA expression and post-translational HNF4α2 protein levels were evaluated by qPCR and Western Blot at 72h. AAV-10A2 and AAV-LKO3 were able to increase HNF4α2 expression with high inter patient variability. (C) The transduction of HNF4α2 by AAV-10A2 and AAV-LKO3 did not increase efficiently downstream transcription and protein expression of albumin nor UGT1A1. This figure is provided for comparison, and shows that treatment of human hepatocytes with an AAV vector is not effective and highly variable.

FIGS. 13A-13B. Summary comparing the HNF4α2 treatment efficiency between AAV-HNF4α2 and mRNA LNP-mRNA-HNF4α2 treatment of cirrhotic human hepatocytes. Only LNP-mRNA-HNF4α2 treatment significantly increased the activation of downstream genes. A) After transduction of AAV-HNF4α2, post-translational HNF4α2 protein levels were evaluated by Western Blot at peak expression (72h, n=3). AAV-10A2 (SIRION Biotech GmbH) and AAV-LKO3 (Vector Biosystems Inc) were able to increase HNF4α2 expression insignificantly with high inter-patient variability. The AAV transduction of HNF4α2 did not increase the downstream transcription of albumin or UGT1A1 at peak expression (72h, n=2). B). After LNP-mRNA-HNF4α2 treatment, post-translational HNF4α2 protein levels were evaluated Western Blot at peak expression (12h). LNP-mRNA-HNF4α2 was able to increase HNF4α2 expression significantly (*p=0.03, Mann-Whitney test, n=4). The transduction of LNP-mRNA-HNF4α2 did increase downstream transcription of albumin and UGT1A1 significantly (Albumin: *p=0.03, UGT1A1: *p=0.03, Mann-Whitey test, n=4).

FIG. 14 shows an annotated exemplary nucleotide sequence (SEQ ID NO: 18) encoding an HNF4α2 mRNA for use to generate therapeutic mRNA.

FIG. 15 shows an annotated exemplary nucleotide sequence (SEQ ID NO: 23) encoding an HNF4α1 (SEQ ID NO: 21) for use to generate therapeutic mRNA.

FIG. 16 shows an exemplary nucleotide sequence (SEQ ID NO: 22) encoding an HNF4α2 (SEQ ID NO: 1) for use to generate therapeutic mRNA.

FIGS. 17A-17B. Effect of adult (P1)-HNF4α2 and embryonic (P2)— HNF4α8 expression in primary human hepatocytes isolated from explanted liver with cirrhosis and end-stage liver disease. LNP-mRNA-HNF4α2 treatment of cirrhotic human hepatocytes increases the expression of the P1-HNF4a isoform and not the P2-HNF4a isoform. Only the P1-HNF4a isoform leads to an upregulation of downstream hepatocyte-expressed genes. (A) Western blot analysis of healthy control hepatocytes, cirrhotic human hepatocytes, and cirrhotic human hepatocytes isolated from the liver of a patient (HH121) who underwent liver transplantation for alcohol induced cirrhosis. Treatment with LNP-mRNA-HNF4α2 increases specifically the P1-HNF4a isoform expression not the P2-HNF4a isoform expression. (B) Fluorescence imaging of primary end-stage cirrhotic hepatocytes from a patient (HH130) who underwent liver transplantation for alcoholic cirrhosis transfected with GFP shows a transfection efficiency of 49%. Only transfection with the P1-HNF4a isoform not with the P2-HNF4a isoform leads to downstream mRNA expression of Albumin and CFVII 48 hours after transfection.

FIGS. 18A-18B. HNF4α2 expression and bioactivity using gene transfer with adeno associate virus (AAV) vs supplemental mRNA technology using lipid-nano particles (LNP) in human cirrhotic hepatocytes with end-stage liver disease. Summary comparing the HNF4a treatment efficiency between AAV-HNF4α2 and mRNA LNP-mRNA-HNF4α2 treatment of cirrhotic human hepatocytes. Only LNP-mRNA-HNF4α2 treatment significantly increased the activation of downstream genes. (A) After transduction of AAV-HNF4α2, post-translational HNF4α2 protein levels were evaluated by Western Blot at peak expression (72h, n=3). AAV-10A2 and AAV-LKO3 were able to increase HNF4α2 expression insignificantly with high inter-patient variability. The AAV transduction of HNF4α2 did not increase the downstream transcription of albumin or UGT1A1 at peak expression (72h, n=2). (B) After LNP-mRNA-HNF4α2 treatment, post-translational HNF4α2 protein levels were evaluated Western Blot at peak expression (12h). LNP-mRNA-HNF4α2 was able to increase HNF4α2 expression significantly (*p=0.03, Mann-Whitney test, n=4). The transduction of LNP-mRNA-HNF4α2 did increase downstream transcription of albumin and UGT1A1 significantly (Albumin: *p=0.03, UGT1A1: *p=0.03, Mann-Whitey test, n=4).

FIG. 19. Comparative analysis between mRNA technologies. Summary comparing the efficiency on HNF4α2 downstream genes after the treatment of cirrhotic human hepatocytes with two different mRNA-HNFa4 constructs. Western blot analysis of cirrhotic human hepatocytes isolated from the liver of patients who underwent liver transplantation for Alcoholic/NASH cirrhosis treated with HNF4α2 mRNA from either native (n=5) or optimized mRNA-HNF4α2 (SEQ ID NO: 19) (n=4). A greater relative expression of HNF4α2 and downstream proteins (albumin and UGT1A1) were observed using the optimized mRNA.

FIG. 20. Amino acid sequence of Homo sapiens hepatocyte nuclear factor 4 alpha (HNF4α), transcript variant 1 (NCBI Reference Sequence: NM_178849.3) and Homo sapiens hepatocyte nuclear factor 4 alpha (HNF4α), transcript variant 2 (NCBI Reference Sequence: NM_000457.6).

FIG. 21. Native (NCBI Reference Sequence: NM_178849.3) coding sequence for HNF4α1 for use to generate therapeutic mRNA.

FIG. 22. Native (NCBI Reference Sequence: NM_000457.6) coding sequence for HNF4α2 for use to generate therapeutic mRNA. The protein-coding sequence is highlighted in light gray.

### SEQUENCES

The nucleic and amino acid sequences listed are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

SEQ ID NO: 1 is the amino acid sequence of HNF4α2.

SEQ ID NO: 2 is an exemplary codon-optimized mRNA encoding HNF4α2.

SEQ ID NO: 3-7 are exemplary amino acid sequences of signal peptides.

SEQ ID NOs: 8-10 are the nucleic acid sequence (RNA) of exemplary 5′ untranslated regions (UTRs).

SEQ ID NOs: 11-17 are the nucleic acid sequence (RNA) of exemplary 3′ untranslated regions (UTRs).

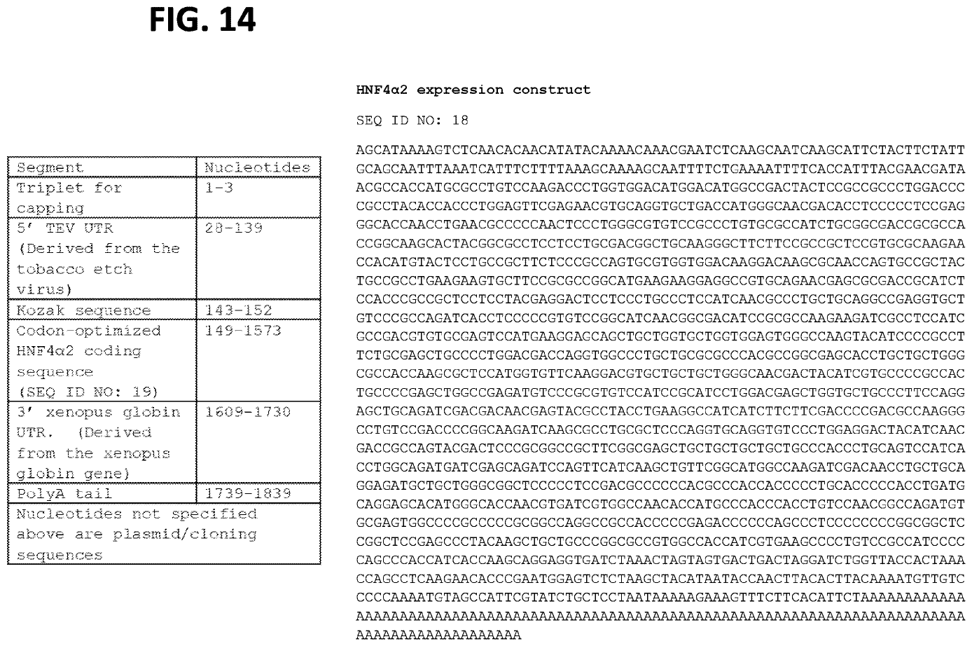

SEQ ID NO: 18 is an exemplary recombinant DNA construct including the HNF4α2 encoding sequence of SEQ ID NO: 19, see also FIG. 14.

SEQ ID NO: 19 is a codon-optimized cDNA encoding HNF4α2 and corresponding to the RNA sequence of SEQ ID NO: 2.

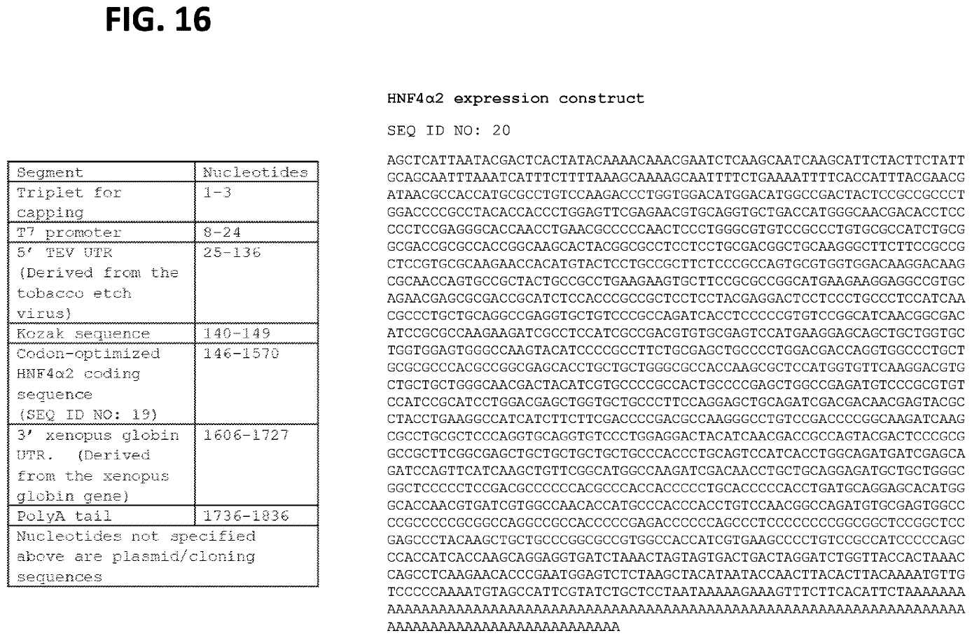

SEQ ID NO: 20 is an exemplary recombinant DNA construct including the HNF4α2 encoding sequence of SEQ ID NO: 19, see also FIG. 16.

SEQ ID NO: 21 is the amino acid sequence of HNF4α1.

SEQ ID NO: 22 is an exemplary native mRNA sequence encoding HNF4α1.

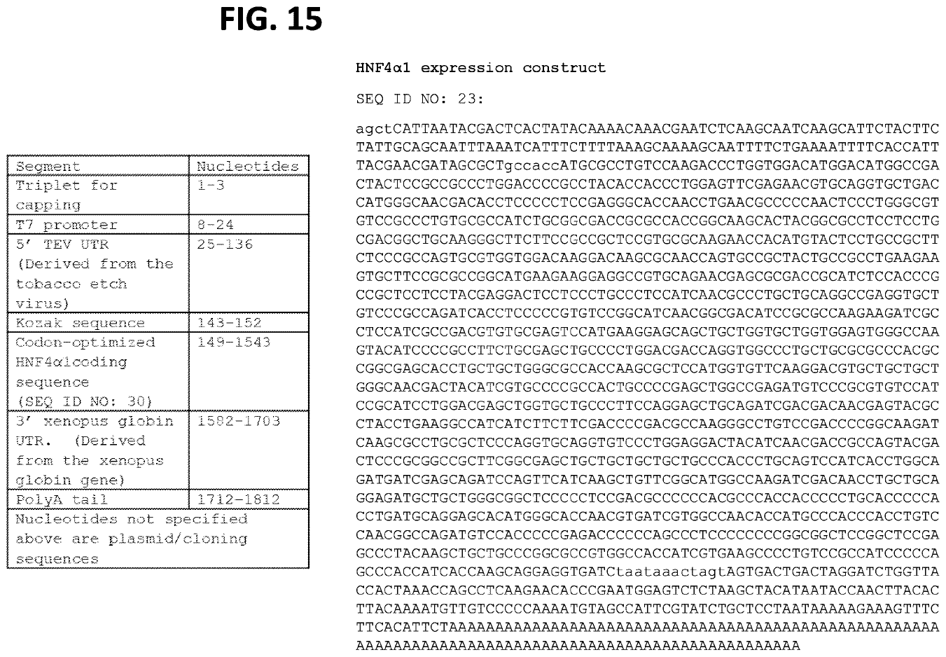

SEQ ID NO: 23 is an exemplary recombinant DNA construct including the HNF4α1 encoding sequence of SEQ ID NO: 30, see also FIG. 15.

SEQ ID NO: 24 is a nucleic acid sequence (mRNA) of an exemplary 5′UTR.

SEQ ID NO: 25 is a nucleic acid sequence (mRNA) of exemplary 3′ UTR.

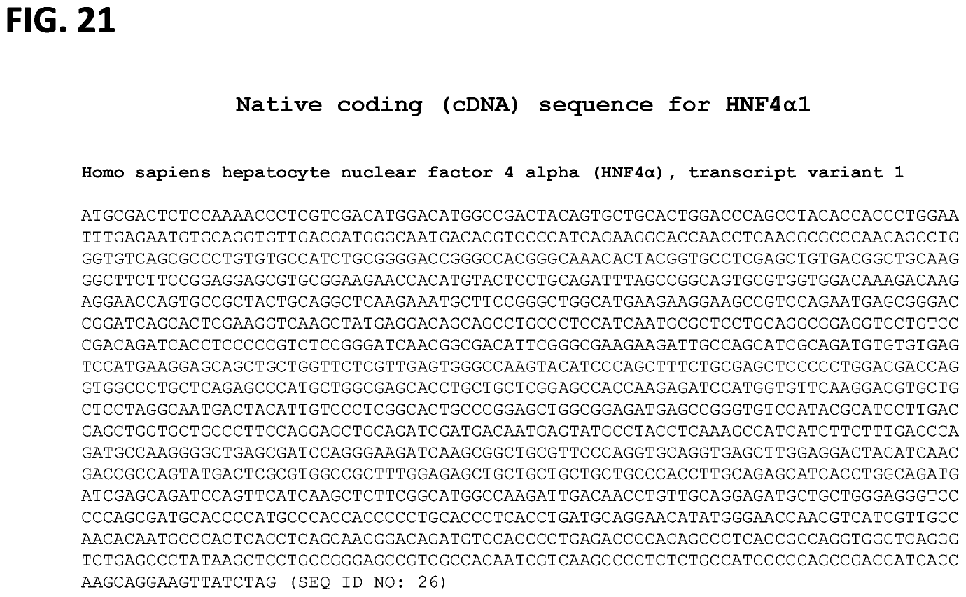

SEQ ID NO: 26 is a cDNA sequence encoding HNF4α1 and corresponding to the RNA sequence of SEQ ID NO: 22, see FIG. 21A, showing native coding sequence.

SEQ ID NO: 27 is an exemplary recombinant RNA including the HNF4α2 encoding sequence of SEQ ID NO: 2 and corresponding to the DNA construct of SEQ ID NO: 18.

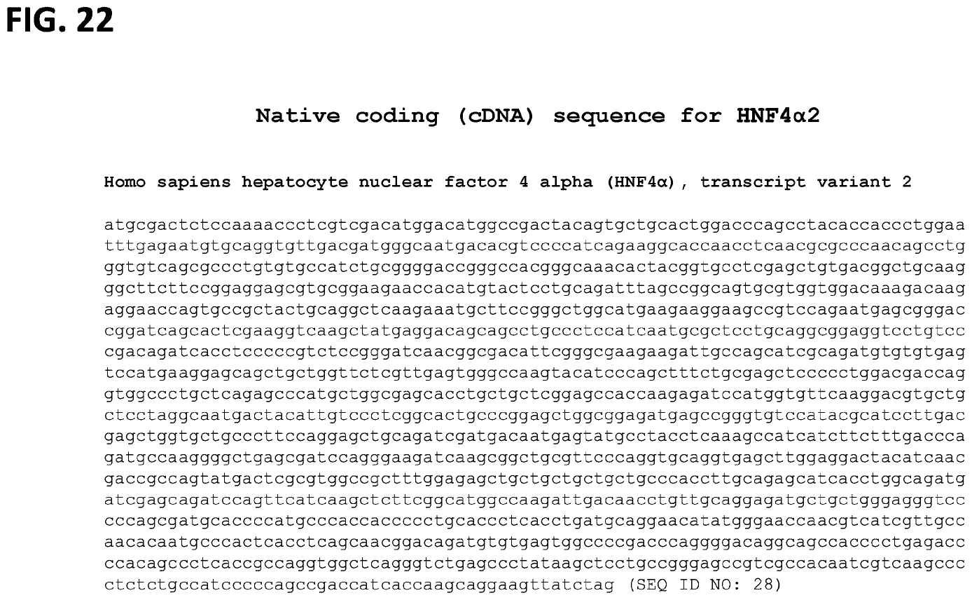

SEQ ID NO: 28 is a nucleic acid sequence encoding HNF4α2, see FIG. 22A showing native coding sequence.

SEQ ID NO: 29 is an exemplary recombinant RNA including the HNF4α2 encoding sequence of SEQ ID NO: 2 and corresponding to the DNA construct of SEQ ID NO: 20.

SEQ ID NO: 30 is an exemplary codon-optimized mRNA encoding HNF4α1.

SEQ ID NO: 31 is a Kozak sequence.

SEQ ID NO: 32 is an exemplary recombinant RNA including the HNF4α1 encoding sequence of SEQ ID NO: 30 and corresponding to the DNA construct of SEQ ID NO: 23.

SEQ ID NO: 33 is a codon-optimized cDNA encoding HNF4α1 and corresponding to the RNA sequence of SEQ ID NO: 30.

### DETAILED DESCRIPTION

In the liver, the transcription factor HNF4a is a key regulator of xenobiotic metabolism, carbohydrate and fatty acid metabolism, bile acid synthesis, blood coagulation, and ureagenesis. As HNF4a is the master transcription factor in the liver, it stabilizes the rest of the hepatic transcriptional network to ensure proper hepatocyte differentiation and function. HNF4a has several isoforms generated through two promoters and alternative splicing. P2-HNF4a isoforms produced from the distal P2 promoter are expressed in fetal liver and are involved in early liver development. P2-HNF4a isoforms are not normally expressed in adult liver, but their aberrant expression has been implicated in the pathogenesis of hepatocellular carcinoma, colorectal carcinoma, and alcoholic hepatitis. P1-HNF4a isoforms produced from the proximal P1 promoter are highly expressed in adult liver and are involved in hepatocyte maturation and function.

The overwhelming majority of HNF4α protein expressed in liver is derived from the P1 promoter which produces the adult HNF4α1, HNF4α2 and to a lesser extent HNF4α3. HNF4α2 is the predominant mRNA isoform in liver. The ratio has been estimated by RT-PCR in rodent hepatocytes (40%-a1:50%-a2:10% α3). Expression of these isoforms is lost along with liver function in ESLD. It is quite difficult to distinguish α1 and α2 isoform proteins by Western blot, as they have only a 10/465 amino acid difference.

Total HNF4α protein expression decreases with chronic liver diseases especially the P1 isoforms (Gunewardena et al., Hepatology 76, 372-386 (2022)). It also is decreased in early stages of liver regeneration after hepatectomy or toxic injury (Huck et al., Hepatology 70, 666-681 (2019)). This is because adult P1 HNF4α is antiproliferative and works against cylinD1 in regulating cell cycle progression (Wu et al., Proc Natl Acad Sci USA 117, 17177-17186 (2020)). HNF4 is also a tumor suppressor for hepato-carcinoma (HCC) (Taniguchi et al., Oncotarget 9, 26144-26156 (2018)) and prostate cancer (Wang et al., Oncogene 39, 1572-1589 (2020)). Adult P1 HNF4α protein expression, or at least activity, must be squelched temporarily so that hepatocytes can proliferate to replace the damaged/injured hepatocytes. In the early stages of chronic liver diseases and in HCC, the P2 promoter is often activated by demethylation due to decreased expression of P1 isoforms which typically repress the P2 promoter (Da et al., Nat Commun 11, 342 (2020)). This is essentially a stress response in the hepatocytes and can facilitate hepatic glucose production with P2 HNF4 isoforms. However, they are ineffective at maintaining mature liver gene expression of CYPs and other metabolic genes.

As disclosed herein, in rodent proof-of-concept studies, upregulating isoform 2 (HNF4α2) in rats with end-stage liver disease completely reversed liver failure and cirrhosis. Moreover, quantitative analysis of gene and protein expression in liver samples from a relatively large cohort of patients with cirrhosis and liver dysfunction demonstrates that loss of HNF4α expression strongly correlates with worsening liver function in humans. An optimized sequence of modified mRNA HNF4α1 was also developed as an alternative therapy.

### I. Terms

Unless otherwise noted, technical terms are used according to conventional usage. Definitions of common terms in molecular biology can be found in Krebs et al (Eds.), Lewin's Genes XII, published by Jones & Bartlett Publishers, 2017; and Meyers et al. (Eds.), The Encyclopedia of Cell Biology and Molecular Medicine, published by Wiley-VCH in 16 volumes, 2008; and other similar references.

As used herein, the singular forms “a,” “an,” and “the” refer to both the singular as well as plural unless the context clearly indicates otherwise. Further, “or” also include “and/or”; thus, “a lentivirus vector or a adenovirus vector” also includes “a lentivirus vector and/or an adenovirus vector,” and compositions of use in the methods herein can be used alone or in combination. Although many methods and materials similar or equivalent to those described herein can be used, particular suitable methods and materials are described below. The term “comprises” means “includes.” As used herein, the transitional phrase consisting essentially of (and grammatical variants) is to be interpreted as encompassing the recited materials or steps and those that do not materially affect the basic and novel characteristic(s) of the recited embodiment. These features are recited in the method embodiments. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure. The term “about” means within five percent, unless otherwise indicated. All publications, patent applications, patents, and other references mentioned herein are incorporated by reference in their entirety. In case of conflict, the present specification, including explanations of terms, will control. In addition, the materials, methods, and examples are illustrative only and not intended to be limiting. To facilitate review of the various aspects, the following explanations of terms are provided:

5′ and/or 3′: Nucleic acid molecules (such as, DNA and RNA) are said to have “5′ ends” and “3′ ends” because mononucleotides are reacted to make polynucleotides in a manner such that the 5′ phosphate of one mononucleotide pentose ring is attached to the 3′ oxygen of its neighbor in one direction via a phosphodiester linkage. Therefore, one end of a linear polynucleotide is referred to as the “5′ end” when its 5′ phosphate is not linked to the 3′ oxygen of a mononucleotide pentose ring. The other end of a polynucleotide is referred to as the “3′ end” when its 3′ oxygen is not linked to a 5′ phosphate of another mononucleotide pentose ring. Notwithstanding that a 5′ phosphate of one mononucleotide pentose ring is attached to the 3′ oxygen of its neighbor, an internal nucleic acid sequence also may be said to have 5′ and 3′ ends.

In either a linear or circular nucleic acid molecule, discrete internal elements are referred to as being “upstream” or 5′ of the “downstream” or 3′ elements. With regard to DNA, this terminology reflects that transcription proceeds in a 5′ to 3′ direction along a DNA strand. Promoter and enhancer elements, which direct transcription of a linked gene, are generally located 5′ or upstream of the coding region. However, enhancer elements can exert their effect even when located 3′ of the promoter element and the coding region. Transcription termination and polyadenylation signals are located 3′ or downstream of the coding region.

Administration: To provide or give a subject an agent, such as a therapeutic agent (e.g. an mRNA encoding HNF4α2), by any effective route. Exemplary routes of administration are described herein.

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein. Codon optimization can be codons that are differentially utilized-represented in genes highly expressed within the human liver compared to the codon usage of the entire coding region of the human genome. A strategy using a maximum amount of liver specific amino acid codons seeks to avoid codons that are under-represented, e.g., because of low quantities of codon matching tRNA in liver cells resulting in slower protein translation, see U.S. Pat. No. 10,898,588.

Conservative variants: “Conservative” amino acid substitutions are those substitutions that do not substantially affect or decrease a function of a protein, such as the ability of the protein to induce an immune response when administered to a subject. The term conservative variation also includes the use of a substituted amino acid in place of an unsubstituted parent amino acid. Furthermore, individual substitutions, deletions or additions which alter, add or delete a single amino acid or a small percentage of amino acids (for instance less than 5%, in some aspects less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the recombinant NiV F ectodomain trimer, such as the ability to induce an immune response when administered to a subject. For instance, if an amino acid residue is essential for a function of the protein, even an otherwise conservative substitution may disrupt that activity. Thus, a conservative substitution does not alter the basic function of a protein of interest.

Control: A reference standard. In some aspects, the control is a negative control sample obtained from a healthy patient. In other aspects, the control is a positive control sample obtained from a patient diagnosed with liver disease. In still other aspects, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Enhancer: A nucleic acid sequence that increases the rate of transcription by increasing the activity of a promoter.

End-Stage Liver disease: A disease of the liver designated by a Child-Pugh score wherein five clinical measures, levels of total bilirubin, serum albumin, prothrombin time prolongation, ascites, and hepatic encephalopathy, are scored using a point system of 1 point, 2 point, and 3 point values for varying levels of each clinical measure, with 3 point values being assigned to the most severe levels of each measure. The total points for all five measures are added to arrive at a Child-Pugh score and classification. Scores of 5-6 designate Child-Pugh Class A, scores of 7-9 designate Child-Pugh Class B, and scores of 10-15 designate Child-Pugh Class C. In general, Child-Pugh Class A indicates the least severe liver disease and Child-Pugh Class C indicates the most severe liver disease. Accordingly, the method disclosed herein can be used to treat a subject having a Child-Pugh Class B or Child-Pugh Class C liver disease. The method disclosed here in can be used to treat a subject having a Child-Pugh Class A liver disease as well. The liver disease includes alcoholic hepatitis and/or or simple accumulation of fat in the hepatocytes (steatosis), macrovesicular steatosis, periportal and lobular inflammation (steatohepatitis), cirrhosis, fibrosis and/or liver ischemia.

Flanking: Near or next to, also, including adjoining, for instance in a linear or circular polynucleotide, such as a DNA molecule.

Gene: A nucleic acid sequence, typically a DNA sequence, that comprises control and coding sequences necessary for the transcription of an RNA, whether an mRNA or otherwise. For instance, a gene may comprise a promoter, one or more enhancers or silencers, a nucleic acid sequence that encodes a RNA and/or a polypeptide, downstream regulatory sequences and, possibly, other nucleic acid sequences involved in regulation of the expression of an mRNA.

As is well known in the art, most eukaryotic genes contain both exons and introns. The term “exon” refers to a nucleic acid sequence found in genomic DNA that is bioinformatically predicted and/or experimentally confirmed to contribute a contiguous sequence to a mature mRNA transcript. The term “intron” refers to a nucleic acid sequence found in genomic DNA that is predicted and/or confirmed not to contribute to a mature mRNA transcript, but rather to be “spliced out” during processing of the transcript.

Hepatocyte: A cell of the main parenchymal tissue of the liver, that make up 70-85% of the mass of the liver. The typical hepatocyte is cubical with sides of 20-30 μm, and produces serum albumin, fibrinogen, and the prothrombin group of clotting factors (except for Factors 3 and 4). Hepatocytes also synthesize lipoproteins, ceruloplasmin, transferrin, complement, and glycoproteins. A hepatocyte is a normal (non-malignant) cell.

Heterologous: A heterologous sequence is a sequence that is not normally (in the wild-type sequence) found adjacent to a second sequence. In one aspect, the sequence is from a different genetic source, such as a virus or organism, than the second sequence. In another aspect, the heterologous sequence is a recombinant sequence that is not normally next to the wild-type sequence.

Hepatocyte nuclear factor (HNF4)α: A polypeptide that, in humans, is encoded by the HNF4A gene. HNF4α is a member of the nuclear receptor superfamily of ligand-dependent transcription factors. Transcription from P1 or P2 promoters combined with alternative splicing potentially generates 12 different transcripts. The method disclosed here involves the supplementation and regulation of HNF4α2. The HNF4α polypeptide is identified in one or more publicly available databases as follows: HGNC: 5024, Entrez Gene: 3172, Ensembl: ENSG00000101076, OMIM: 600281, UniProtKB: P41235, all as available on Dec. 23, 2021. HNF4α isoforms result from both alternate promoter usage and alternative splicing leading to as many as 12 different isoforms of varying transcriptional activity and functionality. HNF4α2 isoform is produced by the proximal P1 promoter. The transcripts expressed from the proximal P1 promoter contain exon 1A on the amino terminus (isoforms α1-a6), whereas those from the distal P2 promoter contain exon 1D (isoforms α7-a12). Exon1A codes for an activation domain (AF1), not present in exon 1D. Furthermore, isoforms α2 and α8 are alternatively spliced variants of isoforms α1 and α7, respectively. Isoforms α2 and α8 contain a 30-bp (10 amino acid) insertion in their 3′ sequence (exon 8) in the F-domain which enhances coactivator binding transcriptional activity. HNF4α2 (variant-2) is the largest transcript and the resulting peptide (474 amino acids) contains both AF1 and AF2 domains. HNF4α2 isoform is a strong transcription inducer of a multitude of genes, the majority of which are involved in cell differentiation, hepatic mature metabolism, protein synthesis and transport of nutrients when compared to other isoforms.

Innate Immune Response: A cellular response to exogenous single stranded nucleic acids, generally of viral or bacterial origin, but can be from any exogenous source, which involves the induction of cytokine expression and release, particularly the interferons, and cell death. Protein synthesis is also reduced during the innate cellular immune response.

Inhibiting or treating a disease: Inhibiting the full development of a disease or condition, for example, in a subject who is at risk for a disease such as end stage liver disease. “Treatment” refers to a therapeutic intervention that ameliorates a sign or symptom of a disease or pathological condition, such as end stage liver disease, after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease, such as when the method increases serum albumin, decreases serum ammonia, improve coagulation activity, decreases ascites production, improves neuropsychological status, decreases bilirubin, improves apolipoprotein and/or improves portal vein or systemic blood flow clearance of cholate. Treatment may be assessed by objective or subjective parameters; including, but not limited to, the results of a physical examination, imaging, or a blood test. A “prophylactic” treatment is a treatment administered to a subject who does not exhibit signs of a disease or exhibits only early signs for the purpose of decreasing the risk of developing pathology, such as to prevent end stage liver disease.

Isolated: An “isolated” biological molecule has been substantially separated, produced apart from, or purified away from other biological molecules in the cell of the organism in which the molecule naturally occurs, such as, other chromosomal and extrachromosomal DNA and RNA, and proteins. Nucleic acids, peptides and proteins that have been “isolated” thus include nucleic acids and proteins purified by standard purification methods. The term also embraces nucleic acids, peptides, and proteins prepared by recombinant expression in a host cell as well as chemically synthesized nucleic acids.

Liver cirrhosis: A chronic disease of the liver marked by a fibrous thickening of the liver tissue histologically, regenerative nodules.

Liver disease: Diseases, disorders, and conditions affecting the liver, and may have a wide range of severity encompassing, for example, simple accumulation of fat in the hepatocytes (steatosis), macrovescicular steatosis, periportal and lobular inflammation (steatohepatitis), cirrhosis, fibrosis, liver ischemia, liver cancer including hepatocellular carcinoma, end-stage liver disease, alcoholic hepatitis and liver failure. End stage liver disease is chronic liver failure. End stage liver disease progresses over time. Most often, chronic liver failure is the result of cirrhosis, a condition in which scar tissue replaces healthy liver tissue until the liver cannot function adequately. Patients with abnormal liver function who develop ascites, variceal hemorrhage, hepatic encephalopathy, or renal impairment are considered to have end-stage liver disease (ESLD). Subjects with end stage liver disease can be identified by Child Pugh scoring, and is designated as Child Pugh class A to C.

Nanoparticle: A particle between 1 and 100 nanometers (nm) in size with a surrounding interfacial layer. The interfacial layer is an integral part of nanoscale matter, fundamentally affecting all of its properties. The interfacial layer typically consists of ions, inorganic and/or organic molecules.

Nucleic acid molecule: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.” Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see for example, Current Protocols in Molecular Biology (Ausubel et al., eds 1995 supplement)).

Nucleotide: This term includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers useful in the methods and compositions of this disclosure are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or composition, including a nucleic acid molecule, capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

A promoter can be a constitutively active promoter (i.e., a promoter that is constitutively in an active/“ON” state), an inducible promoter (i.e., a promoter whose state, active/“ON” or inactive/“OFF”, is controlled by an external stimulus, e.g., the presence of a particular temperature, compound, or protein.), a spatially restricted promoter (e.g., tissue specific promoter, cell type specific promoter, etc.), or it may be a temporally restricted promoter (i.e., the promoter is in the “ON” state or “OFF” state during specific stages of embryonic development or during specific stages of a biological process).

Purified: The term “purified” does not require absolute purity; rather, it is intended as a relative term. Thus, for example, a purified protein preparation is one in which the protein referred to is purer than the protein in its natural environment within a cell. For example, a preparation of a protein is purified such that the protein represents at least 50% of the total protein content of the preparation. Similarly, a purified nucleic acid molecule preparation is one in which the nucleic acid molecule is purer than in an environment including a complex mixture. A purified population of nucleic acids or proteins is greater than about 90%, 91%, 92%, 93%, 94%, 95%, 96%, 97%, 98%, 99% or 100% pure, or free other nucleic acids or proteins, respectively.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences). Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

As used herein, reference to “at least 90% identity” refers to “at least 90%, at least 91%, at least 92%, at least 93%, at least 94%, at least 95%, at least 96%, at least 97%, at least 98%, at least 99%, or even 100% identity” to a specified reference sequence.

Stability (RNA): The extent to which an RNA molecule retains its structural integrity and resists degradation by RNase, and base-catalyzed hydrolysis.

Subject: Human and non-human animals, including all vertebrates, such as mammals and non-mammals, such as non-human primates, mice, rabbits, sheep, dogs, cats, horses, cows, chickens, amphibians, and reptiles. In many aspects of the described methods, the subject is a human.

TATA box: A DNA sequence found in the promoter region of a gene that can be bound by TATA binding protein and transcription factor II D during DNA unwinding and binding by RNA polymerase II. A TATA box sequence typically includes a TATAAA sequence and often includes additional 3′ adenine nucleotides.

Therapeutic mRNA: A ribonucleic acid sequence with defined elements such as 5′ cap, untranslated regions, a gene coding sequence, and a poly adenosine tail that encodes a protein such as HNF4α, which, when delivered to target cells and translated, is able to restore the function of a defective protein, and have a therapeutic effect, such as on the liver.

Transcription factor (TF): A protein that binds to specific DNA sequences and thereby controls the transfer (or transcription) of genetic information from DNA to RNA. TFs perform this function alone or with other proteins in a complex, by promoting (as an activator), or blocking (as a repressor) the recruitment of RNA polymerase (the enzyme that performs the transcription of genetic information from DNA to RNA) to specific genes. The specific DNA sequences to which a TF binds is known as a response element (RE) or regulatory element. Other names include cis-element and cis-acting transcriptional regulatory element.

Transcription factors interact with their binding sites using a combination of electrostatic (of which hydrogen bonds are a special case) and Van der Waals forces. Due to the nature of these chemical interactions, most transcription factors bind DNA in a sequence specific manner. However, not all bases in the transcription factor-binding site may actually interact with the transcription factor. In addition, some of these interactions may be weaker than others. Thus, many transcription factors do not bind just one sequence but are capable of binding a subset of closely related sequences, each with a different strength of interaction.

For example, although the consensus binding site for the TATA-binding protein (TBP) is TATAAAA; however, the TBP transcription factor can also bind similar sequences such as TATATAT or TATATAA.

Transcription factors (TFs) are classified based on many aspects. For example, the secondary, tertiary and quaternary structures of the protein structures DNA-binding sequence and properties, the interaction with the double helix of the DNA, and the metal and other binding characteristics. The JASPAR database and TRANSFAC (TRANSFAC® 7.0 Public 2005) are two web-based transcription factor databases, their experimentally-proven binding sites, and regulated genes. HNF-4 functions as a transcription factor. An exemplary binding site for HNF-4 is provided in Wang et al., “Hepatocyte nuclear factor-4a interacts with other hepatocyte nuclear factors in regulating transthyretin gene expression,” FEBS J., 277(19):4066-75, 2010, incorporated herein by reference.

Untranslated Region: mRNA molecules can have regions of differing sequence located before the translation start codon and after the translation stop codon that are not translated into a protein. These regions, termed the five prime untranslated region (5′ UTR) and three prime untranslated region (3′ UTR), respectively, can affect mRNA stability, mRNA localization, and translational efficiency of the mRNA to which they are joined. UTRs play roles in the post-transcriptional regulation of protein expression. This includes modulating the transport of mRNAs out of the nucleus, translation efficiency, subcellular localization, and stability.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### II. Overview

In some aspects, disclosed is a recombinant mRNA encoding an HNF4α1 or HNF4α2, wherein the recombinant mRNA includes: i) a nucleic acid sequence at least 95% identical to one of a) SEQ ID NO: 2 [HNF4α2], b) SEQ ID NO: 22 [HNFα1]; or c) SEQ ID NO: 30 [HNFα1]. In one aspect, the recombinant mRNA encoding the HNF4α1 is codon optimized. In another aspect, the recombinant mRNA encoding the HNF4α2 is codon optimized. The recombinant RNA also optionally includes one or more of: d) a 5′ capping structure, e) a promoter, f) a nucleic acid molecule encoding a signal peptide, g) a 5″ untranslated region (UTR), h) a 3′ UTR, and h) a polyA tail. In some aspects, the recombinant mRNA includes a nucleic acid sequence at least 95% identical to SEQ ID NO: 2 that encodes SEQ ID NO: 1; a nucleic acid sequence at least 95% identical to SEQ ID NO: 22 that encodes SEQ ID NO: 21; or a nucleic acid sequence at least 95% identical to SEQ ID NO: 30 that encodes SEQ ID NO: 21. In more aspects, the recombinant mRNA includes SEQ ID NO: 2, SEQ ID NO: 22, or SEQ ID NO: 30.

In further aspects, the recombinant mRNA includes the 5′UTR, wherein the 5′ UTR includes the nucleic acid sequence of one of SEQ ID NOs: 8-10 and 24. In yet other aspects, the recombinant mRNA includes the 3′ UTR, and wherein the 3′ UTR includes the nucleic acid sequence of one of SEQ ID NOs: 11-17 and 25. In some aspects, the recombinant mRNA includes the nucleic acid molecule encoding the signal peptide, wherein the signal peptide includes the amino acid sequence of one of SEQ ID NOs: 3-7.

In some aspects, the recombinant mRNA comprises a nucleic acid sequence set forth as any one of SEQ ID NOs: 18, 20, or 23.

In more aspects, the recombinant mRNA includes an 5′ capping structure that is a 7mG(5′)ppp(5′)N1mpNp cap at the 5′ end of the 5′UTR.

In other aspects, the recombinant mRNA includes a modified nucleotide that increases half-life of the recombinant mRNA, increases protein production, and/or reduces induction of innate immunity. In some aspects, the modified nucleotide is modified uridine or a pseudouridine. In more aspects, the pseudouridine is 1-methylpseudouridine.

In some aspects, a lipid nanoparticle is disclosed that includes the recombinant mRNA. The lipid nanoparticle can include polyethylene glycol (PEG)-modified lipid, a non-cationic lipid, a sterol, an ionizable cationic lipid, or any combination thereof. In some aspects, the PEG has an average molecular weight of about 20000 (PEG2000 DMG). In additional aspects, the lipid nanoparticle comprises the ionizable lipid. In further aspects, the lipid nanoparticle includes (2 hydroxyethyl)(6 oxo 6-(undecycloxy)hexyl)amino)octanoate, 1,2 distearoyl sn glycerol-3 phosphocholine (DSPC), cholesterol, and 1-monomethoxypolyethyleneglycol-2,3,dimyristylglycerol.

Methods are also disclosed for treating liver disease or end-stage liver failure in a subject, that include administering to the subject a therapeutically effective amount of a recombinant mRNA encoding HNF4α1. In some aspects, the method increases serum albumin, decreases serum ammonia, improve coagulation activity, decreases ascites production, improves neuropsychological status, decreases bilirubin, improves apolipoprotein and/or improves portal vein or systemic blood flow clearance of cholate in the subject. In more aspects, the subject is human. In further aspects, the recombinant mRNA encodes a signal peptide fused to the HNF4α1. In more aspects, the signal peptide includes the amino acid sequence of one of SEQ ID NOs: 3-7. In further aspects, the recombinant mRNA is codon-optimized for expression in human cells.

In some aspects, the recombinant mRNA includes a nucleic acid sequence is at least 80% identical to SEQ ID NO: 22 or 30 and encodes HNF4α1. In additional aspects, the recombinant mRNA includes a nucleic acid sequence is at least 95% identical to SEQ ID NO: 22 or 30 and encodes HNF4α2. In further aspects, the recombinant mRNA comprises SEQ ID NO: 22 or 30. In additional aspects, the recombinant mRNA comprises a 5′ untranslated region (UTR). In more aspects, the 5′ untranslated region includes the nucleic acid sequence of one of SEQ ID NOs: 8-10 and 24. In additional aspects, the recombinant mRNA further comprises a 7mG(5′)ppp(5′)N1mpNp cap at the 5′ end of the 5′UTR. In some aspects, the recombinant mRNA comprises a 3′ UTR. In more aspects, the 3′ UTR includes the nucleic acid sequence of one of SEQ ID NOs: 11-17 and 25. In additional aspects, the recombinant mRNA comprises a modified nucleotide increases half-life of the recombinant mRNA, increases protein production, and/or reduces induction of innate immunity.

The modified nucleotide can be a modified uridine or a pseudouridine, such as 1-methylpseudouridine.

In some aspects, the method includes administering to the subject a therapeutically effective amount of a lipid nanoparticle comprising the recombinant mRNA. The lipid nanoparticle can include a polyethylene glycol (PEG)-modified lipid, a non-cationic lipid, a sterol, an ionizable cationic lipid, or any combination thereof. In more aspects, the PEG has an average molecular weight of about 20000 (PEG2000 DMG). In further aspects, the lipid nanoparticle includes an ionizable lipid. In more aspects, the lipid nanoparticle includes (2 hydroxyethyl)(6 oxo 6-(undecycloxy)hexyl)amino)octanoate, 1,2 distearoyl sn glycerol-3 phosphocholine (DSPC), cholesterol, and 1-monomethoxypolyethyleneglycol-2,3,dimyristylglycerol.

In further aspects, the method includes administering to the subject a composition comprising the therapeutically effective amount of the recombinant mRNA and a pharmaceutically acceptable carrier. The composition can include the lipid nanoparticle comprising the recombinant mRNA. In addition, the pharmaceutically acceptable carrier can include a sterile buffer and a stabilizing agent. In some aspects, the composition comprises trometamol (Tris) buffer, sucrose, and sodium acetate, at physiological pH.

In some aspects, the liver disease is liver cirrhosis or end-stage liver failure. In further aspects, the liver disease is a degenerative liver disease. In more aspects, wherein the degenerative liver disease is non-alcoholic fatty liver disease (NASH) or alcohol related liver disease (ALD). In some aspects, the subject has liver disease with a Child-Pugh score level of A, B or C.

Methods are disclosed herein for treating end-stage liver failure in a subject. These methods include administering to the subject a therapeutically effective amount of a recombinant mRNA encoding HNF4α2, thereby treating the end-stage liver failure in the subject. In some aspects, the method increases serum albumin, decreases serum and arterial ammonia, improve coagulation activity, decreases ascites production, improves neuropsychological status, decreases bilirubin, improves apolipoprotein and/or improves portal vein or systemic blood flow clearance of cholate in the subject. In some aspects, the subject is mammal, e.g. a human.

In some aspects, the recombinant mRNA encodes a signal peptide fused to the HNF4α2. In non-limiting examples, signal peptide can include the amino acid sequence of one of SEQ ID NOs: 3-7.

In more aspects, the recombinant mRNA can be codon-optimized for expression in human cells. In further aspects, the recombinant mRNA can include a nucleic acid sequence is at least 80% identical to SEQ ID NO: 2 and encodes HNF4α2. In other aspects, the recombinant mRNA includes a nucleic acid sequence is at least 95% identical to SEQ ID NO: 2 and encodes HNF4α2. In other aspects, the recombinant mRNA includes SEQ ID NO: 2.

In further aspects, the recombinant mRNA includes a 5′ untranslated region (UTR). In some non-limiting examples, the 5′ untranslated region includes the nucleic acid sequence of one of SEQ ID NOs: 8-10. In some aspects, the recombinant mRNA further includes a 7mG(5′)ppp(5′)N1mpNp cap at the 5′ end of the 5′UTR.

In more aspects, the recombinant mRNA includes a 3′ UTR. In some non-limiting examples, the 3′ UTR comprises the nucleic acid sequence of one of SEQ ID NOs: 11-17 and 25.

In aspects, the recombinant mRNA includes a modified nucleotide increases half-life of the recombinant mRNA, increases protein production, and/or reduces induction of innate immunity. In further aspects, the modified nucleotide is modified uridine or a pseudouridine. In some non-limiting examples, the modified nucleotide is 1-methylpseudouridine.

In more aspects, the method can include administering to the subject a therapeutically effective amount of a lipid nanoparticle comprising the recombinant mRNA. In aspects, the lipid nanoparticle includes a polyethylene glycol (PEG)-modified lipid, a non-cationic lipid, a sterol, an ionizable cationic lipid, or any combination thereof. In some non-limiting examples, the PEG has an average molecular weight of about 20000 (PEG2000 DMG). In other aspects, the lipid nanoparticle includes the ionizable lipid. In further aspects, the lipid nanoparticle comprises (2 hydroxyethyl)(6 oxo 6-(undecycloxy)hexyl)amino)octanoate, 1,2 distearoyl sn glycerol-3 phosphocholine (DSPC), cholesterol, and 1-monomethoxypolyethyleneglycol-2,3,dimyristylglycerol.

In some aspects, the method includes administering to the subject a composition including the therapeutically effective amount of the recombinant mRNA and a pharmaceutically acceptable carrier. The composition can include the lipid nanoparticle comprising the recombinant mRNA. In some aspects, the pharmaceutical composition includes a pharmaceutically acceptable carrier comprises a sterile buffer and a stabilizing agent. In additional aspects, the composition comprises trometamol (Tris) buffer, sucrose, and sodium acetate, at physiological pH.

### II. Recombinant mRNA Encoding HNF4α2 or HNF4α1

### HNF4α2

In some aspects, the presently disclosed methods utilize a recombinant mRNA encoding HNF4α2. In one aspect, an mRNA of use includes an in vitro-transcribed nucleic acid. The RNA is produced by in vitro transcription using a plasmid DNA template generated synthetically. DNA of interest from any source can be directly converted by PCR into a template for in vitro mRNA synthesis using appropriate primers and RNA polymerase. In various aspects, plasmid is used to generate a template for in vitro transcription of mRNA which is used in the disclosed methods.

An exemplary amino acid sequence of HNF4α2 is provided as:

(SEQ ID NO: 1)
MRLSKTLVDMDMADYSAALDPAYTTLEFENVQVLTMGNDTSPSEGTNLNAPNSLGVSALCAICGDRATGKHY
GASSCDGCKGFFRRSVRKNHMYSCRFSRQCVVDKDKRNQCRYCRLKKCFRAGMKKEAVQNERDRISTRRSSY
EDSSLPSINALLQAEVLSRQITSPVSGINGDIRAKKIASIADVCESMKEQLLVLVEWAKYIPAFCELPLDDQ
VALLRAHAGEHLLLGATKRSMVFKDVLLLGNDYIVPRHCPELAEMSRVSIRILDELVLPFQELQIDDNEYAY
LKAIIFFDPDAKGLSDPGKIKRLRSQVQVSLEDYINDRQYDSRGRFGELLLLLPTLQSITWQMIEQIQFIKL
FGMAKIDNLLQEMLLGGSPSDAPHAHHPLHPHLMQEHMGTNVIVANTMPTHLSNGQMCEWPRPRGQAATPET
PQPSPPGGSGSEPYKLLPGAVATIVKPLSAIPQPTITKQEVI

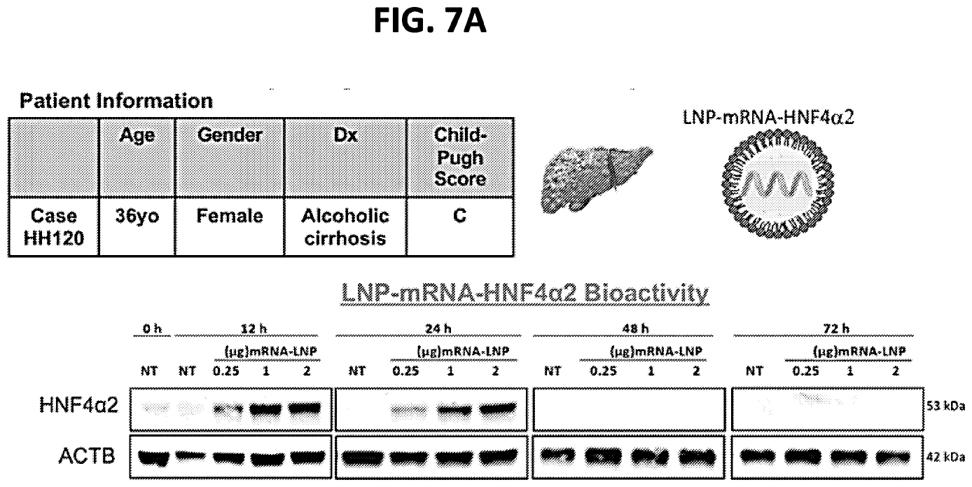

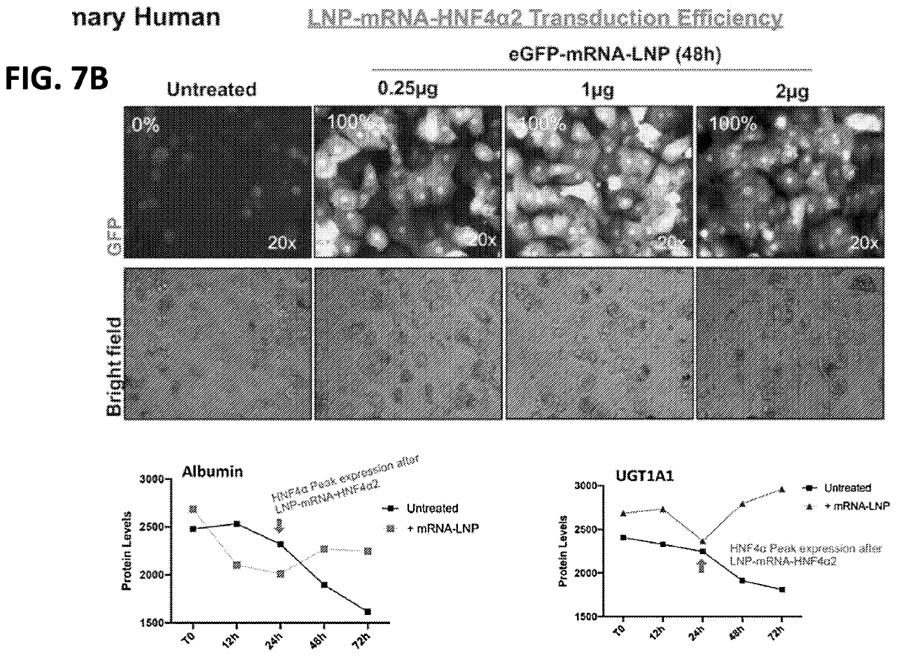

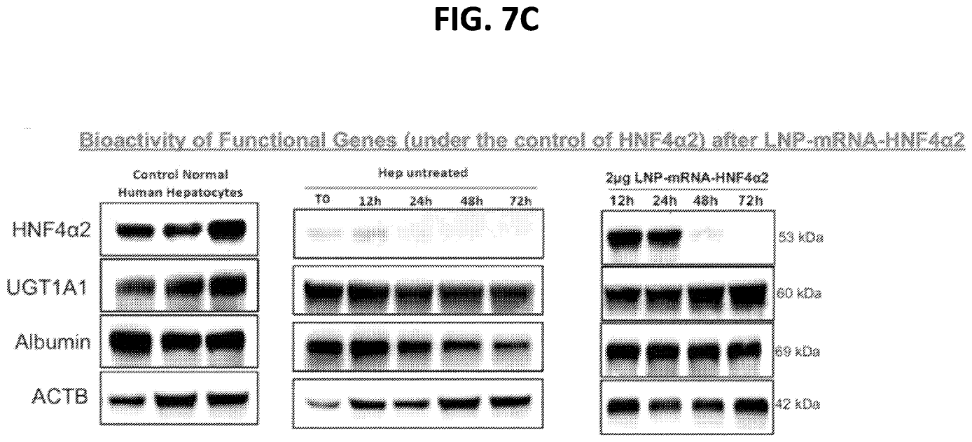

See also GENBANK Accession No. NM_000457.4, as available on Dec. 27, 2021, incorporated herein by reference, and FIG. 7 of U.S. Patent No. U.S. Pat. No. 9,981,048, incorporated herein by reference.

In some aspects, an mRNA of use in the disclosed methods encodes a polypeptide at least 85%, 90%, 95%, 96%, 97%, 98% or 99% identical to SEQ ID NO: 1. In further aspects, an mRNA of use in the disclosed methods encodes a polypeptide set forth as SEQ ID NO: 1. In yet other aspects, an mRNA of use in the disclosed methods encodes a polypeptide with at most 1, 2, 3, 4, or 5 conservative substitutions in SEQ ID NO: 1.

A codon optimized mRNA encoding the HNF4α2 protein of SEQ ID NO: 1 is provided below:

(SEQ ID NO: 2)
AUGCGCCUGUCCAAGACCCUGGUGGACAUGGACAUGGCCGACUACUCCGCCGCCCUGGACCCCGCCUACACC
ACCCUGGAGUUCGAGAACGUGCAGGUGCUGACCAUGGGCAACGACACCUCCCCCUCCGAGGGCACCAACCUG
AACGCCCCCAACUCCCUGGGCGUGUCCGCCCUGUGCGCCAUCUGCGGCGACCGCGCCACCGGCAAGCACUAC
GGCGCCUCCUCCUGCGACGGCUGCAAGGGCUUCUUCCGCCGCUCCGUGCGCAAGAACCACAUGUACUCCUGC
CGCUUCUCCCGCCAGUGCGUGGUGGACAAGGACAAGCGCAACCAGUGCCGCUACUGCCGCCUGAAGAAGUGC
UUCCGCGCCGGCAUGAAGAAGGAGGCCGUGCAGAACGAGCGCGACCGCAUCUCCACCCGCCGCUCCUCCUAC
GAGGACUCCUCCCUGCCCUCCAUCAACGCCCUGCUGCAGGCCGAGGUGCUGUCCCGCCAGAUCACCUCCCCC
GUGUCCGGCAUCAACGGCGACAUCCGCGCCAAGAAGAUCGCCUCCAUCGCCGACGUGUGCGAGUCCAUGAAG
GAGCAGCUGCUGGUGCUGGUGGAGUGGGCCAAGUACAUCCCCGCCUUCUGCGAGCUGCCCCUGGACGACCAG
GUGGCCCUGCUGCGCGCCCACGCCGGCGAGCACCUGCUGCUGGGCGCCACCAAGCGCUCCAUGGUGUUCAAG
GACGUGCUGCUGCUGGGCAACGACUACAUCGUGCCCCGCCACUGCCCCGAGCUGGCCGAGAUGUCCCGCGUG
UCCAUCCGCAUCCUGGACGAGCUGGUGCUGCCCUUCCAGGAGCUGCAGAUCGACGACAACGAGUACGCCUAC
CUGAAGGCCAUCAUCUUCUUCGACCCCGACGCCAAGGGCCUGUCCGACCCCGGCAAGAUCAAGCGCCUGCGC
UCCCAGGUGCAGGUGUCCCUGGAGGACUACAUCAACGACCGCCAGUACGACUCCCGCGGCCGCUUCGGCGAG
CUGCUGCUGCUGCUGCCCACCCUGCAGUCCAUCACCUGGCAGAUGAUCGAGCAGAUCCAGUUCAUCAAGCUG
UUCGGCAUGGCCAAGAUCGACAACCUGCUGCAGGAGAUGCUGCUGGGCGGCUCCCCCUCCGACGCCCCCCAC
GCCCACCACCCCCUGCACCCCCACCUGAUGCAGGAGCACAUGGGCACCAACGUGAUCGUGGCCAACACCAUG
CCCACCCACCUGUCCAACGGCCAGAUGUGCGAGUGGCCCCGCCCCCGCGGCCAGGCCGCCACCCCCGAGACC
CCCCAGCCCUCCCCCCCCGGCGGCUCCGGCUCCGAGCCCUACAAGCUGCUGCCCGGCGCCGUGGCCACCAUC
GUGAAGCCCCUGUCCGCCAUCCCCCAGCCCACCAUCACCAAGCAGGAGGUGAUCUAA

In some aspects, an mRNA sequence encoding HNF4α2 is at least about 80% identical to SEQ ID NO: 2. In other aspects, an mRNA encoding HNF4α2 is at least about 85%, 90%, 95%, 96%, 97%, 98% or 99% identical to SEQ ID NO: 2. In further aspects, the mRNA encoding HNF4α2 is at least about 95%, 96%, 97%, 98% or 99% identical to SEQ ID NO: 2. In some aspects, an mRNA sequence encoding HNF4α2 is at least about 80% identical to SEQ ID NO: 2 and encodes SEQ ID NO: 1. In other aspects, an mRNA encoding HNF4α2 is at least about 85%, 90%, 95%, 96%, 97%, 98% or 99% identical to SEQ ID NO: 2 and encodes SEQ ID NO: 1. In further aspects, the mRNA encoding HNF4α2 is at least about 95%, 96%, 97%, 98% or 99% identical to SEQ ID NO: 2 and encodes SEQ ID NO: 1.

A codon-optimized cDNA encoding the HNF4α isoform 2 protein of SEQ ID NO: 1 and corresponding to the RNA sequence of SEQ ID NO: 2 is provided as SEQ ID NO: 19:

(SEQ ID NO: 19)
ATGCGCCTGTCCAAGACCCTGGTGGACATGGACATGGCCGACTACTCCGCCGCCCTGGACCCCGCCTACACC
ACCCTGGAGTTCGAGAACGTGCAGGTGCTGACCATGGGCAACGACACCTCCCCCTCCGAGGGCACCAACCTG
AACGCCCCCAACTCCCTGGGCGTGTCCGCCCTGTGCGCCATCTGCGGCGACCGCGCCACCGGCAAGCACTAC
GGCGCCTCCTCCTGCGACGGCTGCAAGGGCTTCTTCCGCCGCTCCGTGCGCAAGAACCACATGTACTCCTGC
CGCTTCTCCCGCCAGTGCGTGGTGGACAAGGACAAGCGCAACCAGTGCCGCTACTGCCGCCTGAAGAAGTGC
TTCCGCGCCGGCATGAAGAAGGAGGCCGTGCAGAACGAGCGCGACCGCATCTCCACCCGCCGCTCCTCCTAC
GAGGACTCCTCCCTGCCCTCCATCAACGCCCTGCTGCAGGCCGAGGTGCTGTCCCGCCAGATCACCTCCCCC
GTGTCCGGCATCAACGGCGACATCCGCGCCAAGAAGATCGCCTCCATCGCCGACGTGTGCGAGTCCATGAAG
GAGCAGCTGCTGGTGCTGGTGGAGTGGGCCAAGTACATCCCCGCCTTCTGCGAGCTGCCCCTGGACGACCAG
GTGGCCCTGCTGCGCGCCCACGCCGGCGAGCACCTGCTGCTGGGCGCCACCAAGCGCTCCATGGTGTTCAAG
GACGTGCTGCTGCTGGGCAACGACTACATCGTGCCCCGCCACTGCCCCGAGCTGGCCGAGATGTCCCGCGTG
TCCATCCGCATCCTGGACGAGCTGGTGCTGCCCTTCCAGGAGCTGCAGATCGACGACAACGAGTACGCCTAC
CTGAAGGCCATCATCTTCTTCGACCCCGACGCCAAGGGCCTGTCCGACCCCGGCAAGATCAAGCGCCTGCGC
TCCCAGGTGCAGGTGTCCCTGGAGGACTACATCAACGACCGCCAGTACGACTCCCGCGGCCGCTTCGGCGAG
CTGCTGCTGCTGCTGCCCACCCTGCAGTCCATCACCTGGCAGATGATCGAGCAGATCCAGTTCATCAAGCTG
TTCGGCATGGCCAAGATCGACAACCTGCTGCAGGAGATGCTGCTGGGCGGCTCCCCCTCCGACGCCCCCCAC
GCCCACCACCCCCTGCACCCCCACCTGATGCAGGAGCACATGGGCACCAACGTGATCGTGGCCAACACCATG
CCCACCCACCTGTCCAACGGCCAGATGTGCGAGTGGCCCCGCCCCCGCGGCCAGGCCGCCACCCCCGAGACC
CCCCAGCCCTCCCCCCCCGGCGGCTCCGGCTCCGAGCCCTACAAGCTGCTGCCCGGCGCCGTGGCCACCATC
GTGAAGCCCCTGTCCGCCATCCCCCAGCCCACCATCACCAAGCAGGAGGTGATCTAA

In some aspects, the cDNA sequence of SEQ ID NO: 19 is used for the manufacture of RNA construct encoding SEQ ID NO: 1. In other aspects, an mRNA encoding HNF4α2 is provided that has an equivalent cDNA sequence at least about 95%, 96%, 97%, 98% or 99% identical to SEQ ID NO: 19.

An exemplary DNA sequence (SEQ ID NO: 18) encoding HNF4α2 that can be converted to mRNA for use in the disclosed methods is shown below, and in FIG. 14 with the segments of the sequence identified in the figure. In some aspects, a nucleic acid construct comprising a sequence set forth as SEQ ID NO: 18 is provided.

(SEQ ID NO: 18)
AGCATAAAAGTCTCAACACAACATATACAAAACAAACGAATCTCAAGCAATCAAGCATTCTACTTCTATTGC
AGCAATTTAAATCATTTCTTTTAAAGCAAAAGCAATTTTCTGAAAATTTTCACCATTTACGAACGATAACGC
CACCATGCGCCTGTCCAAGACCCTGGTGGACATGGACATGGCCGACTACTCCGCCGCCCTGGACCCCGCCTA
CACCACCCTGGAGTTCGAGAACGTGCAGGTGCTGACCATGGGCAACGACACCTCCCCCTCCGAGGGCACCAA
CCTGAACGCCCCCAACTCCCTGGGCGTGTCCGCCCTGTGCGCCATCTGCGGCGACCGCGCCACCGGCAAGCA
CTACGGCGCCTCCTCCTGCGACGGCTGCAAGGGCTTCTTCCGCCGCTCCGTGCGCAAGAACCACATGTACTC
CTGCCGCTTCTCCCGCCAGTGCGTGGTGGACAAGGACAAGCGCAACCAGTGCCGCTACTGCCGCCTGAAGAA
GTGCTTCCGCGCCGGCATGAAGAAGGAGGCCGTGCAGAACGAGCGCGACCGCATCTCCACCCGCCGCTCCTC
CTACGAGGACTCCTCCCTGCCCTCCATCAACGCCCTGCTGCAGGCCGAGGTGCTGTCCCGCCAGATCACCTC
CCCCGTGTCCGGCATCAACGGCGACATCCGCGCCAAGAAGATCGCCTCCATCGCCGACGTGTGCGAGTCCAT
GAAGGAGCAGCTGCTGGTGCTGGTGGAGTGGGCCAAGTACATCCCCGCCTTCTGCGAGCTGCCCCTGGACGA
CCAGGTGGCCCTGCTGCGCGCCCACGCCGGCGAGCACCTGCTGCTGGGCGCCACCAAGCGCTCCATGGTGTT
CAAGGACGTGCTGCTGCTGGGCAACGACTACATCGTGCCCCGCCACTGCCCCGAGCTGGCCGAGATGTCCCG
CGTGTCCATCCGCATCCTGGACGAGCTGGTGCTGCCCTTCCAGGAGCTGCAGATCGACGACAACGAGTACGC
CTACCTGAAGGCCATCATCTTCTTCGACCCCGACGCCAAGGGCCTGTCCGACCCCGGCAAGATCAAGCGCCT
GCGCTCCCAGGTGCAGGTGTCCCTGGAGGACTACATCAACGACCGCCAGTACGACTCCCGCGGCCGCTTCGG
CGAGCTGCTGCTGCTGCTGCCCACCCTGCAGTCCATCACCTGGCAGATGATCGAGCAGATCCAGTTCATCAA
GCTGTTCGGCATGGCCAAGATCGACAACCTGCTGCAGGAGATGCTGCTGGGCGGCTCCCCCTCCGACGCCCC
CCACGCCCACCACCCCCTGCACCCCCACCTGATGCAGGAGCACATGGGCACCAACGTGATCGTGGCCAACAC
CATGCCCACCCACCTGTCCAACGGCCAGATGTGCGAGTGGCCCCGCCCCCGCGGCCAGGCCGCCACCCCCGA
GACCCCCCAGCCCTCCCCCCCCGGCGGCTCCGGCTCCGAGCCCTACAAGCTGCTGCCCGGCGCCGTGGCCAC
CATCGTGAAGCCCCTGTCCGCCATCCCCCAGCCCACCATCACCAAGCAGGAGGTGATCTAAACTAGTAGTGA
CTGACTAGGATCTGGTTACCACTAAACCAGCCTCAAGAACACCCGAATGGAGTCTCTAAGCTACATAATACC
AACTTACACTTACAAAATGTTGTCCCCCAAAATGTAGCCATTCGTATCTGCTCCTAATAAAAAGAAAGTTTC
TTCACATTCTAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAA

In some aspects, the DNA sequence of SEQ ID NO: 18 is used for the manufacture of therapeutic RNA construct encoding HNF4α2, for example, for use in the disclosed methods. In some aspects, a therapeutic RNA comprising the nucleotide sequence set forth as SEQ ID NO: 27, which corresponds to the DNA sequence of SEQ ID NO: 18, is provided.

(SEQ ID NO: 27)
AGCAUAAAAGUCUCAACACAACAUAUACAAAACAAACGAAUCUCAAGCAAUCAAGCAUUCUACUUCUAUUGC
AGCAAUUUAAAUCAUUUCUUUUAAAGCAAAAGCAAUUUUCUGAAAAUUUUCACCAUUUACGAACGAUAACGC
CACCAUGCGCCUGUCCAAGACCCUGGUGGACAUGGACAUGGCCGACUACUCCGCCGCCCUGGACCCCGCCUA
CACCACCCUGGAGUUCGAGAACGUGCAGGUGCUGACCAUGGGCAACGACACCUCCCCCUCCGAGGGCACCAA
CCUGAACGCCCCCAACUCCCUGGGCGUGUCCGCCCUGUGCGCCAUCUGCGGCGACCGCGCCACCGGCAAGCA
CUACGGCGCCUCCUCCUGCGACGGCUGCAAGGGCUUCUUCCGCCGCUCCGUGCGCAAGAACCACAUGUACUC
CUGCCGCUUCUCCCGCCAGUGCGUGGUGGACAAGGACAAGCGCAACCAGUGCCGCUACUGCCGCCUGAAGAA
GUGCUUCCGCGCCGGCAUGAAGAAGGAGGCCGUGCAGAACGAGCGCGACCGCAUCUCCACCCGCCGCUCCUC
CUACGAGGACUCCUCCCUGCCCUCCAUCAACGCCCUGCUGCAGGCCGAGGUGCUGUCCCGCCAGAUCACCUC
CCCCGUGUCCGGCAUCAACGGCGACAUCCGCGCCAAGAAGAUCGCCUCCAUCGCCGACGUGUGCGAGUCCAU
GAAGGAGCAGCUGCUGGUGCUGGUGGAGUGGGCCAAGUACAUCCCCGCCUUCUGCGAGCUGCCCCUGGACGA
CCAGGUGGCCCUGCUGCGCGCCCACGCCGGCGAGCACCUGCUGCUGGGCGCCACCAAGCGCUCCAUGGUGUU
CAAGGACGUGCUGCUGCUGGGCAACGACUACAUCGUGCCCCGCCACUGCCCCGAGCUGGCCGAGAUGUCCCG
CGUGUCCAUCCGCAUCCUGGACGAGCUGGUGCUGCCCUUCCAGGAGCUGCAGAUCGACGACAACGAGUACGC
CUACCUGAAGGCCAUCAUCUUCUUCGACCCCGACGCCAAGGGCCUGUCCGACCCCGGCAAGAUCAAGCGCCU
GCGCUCCCAGGUGCAGGUGUCCCUGGAGGACUACAUCAACGACCGCCAGUACGACUCCCGCGGCCGCUUCGG
CGAGCUGCUGCUGCUGCUGCCCACCCUGCAGUCCAUCACCUGGCAGAUGAUCGAGCAGAUCCAGUUCAUCAA
GCUGUUCGGCAUGGCCAAGAUCGACAACCUGCUGCAGGAGAUGCUGCUGGGCGGCUCCCCCUCCGACGCCCC
CCACGCCCACCACCCCCUGCACCCCCACCUGAUGCAGGAGCACAUGGGCACCAACGUGAUCGUGGCCAACAC
CAUGCCCACCCACCUGUCCAACGGCCAGAUGUGCGAGUGGCCCCGCCCCCGCGGCCAGGCCGCCACCCCCGA
GACCCCCCAGCCCUCCCCCCCCGGCGGCUCCGGCUCCGAGCCCUACAAGCUGCUGCCCGGCGCCGUGGCCAC
CAUCGUGAAGCCCCUGUCCGCCAUCCCCCAGCCCACCAUCACCAAGCAGGAGGUGAUCUAAACUAGUAGUGA
CUGACUAGGAUCUGGUUACCACUAAACCAGCCUCAAGAACACCCGAAUGGAGUCUCUAAGCUACAUAAUACC
AACUUACACUUACAAAAUGUUGUCCCCCAAAAUGUAGCCAUUCGUAUCUGCUCCUAAUAAAAAGAAAGUUUC
UUCACAUUCUAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAA

Another exemplary DNA sequence (SEQ ID NO: 20) encoding HNF4α2 that can be converted to mRNA for use in the disclosed methods is shown below and in FIG. 16, with the segments of the sequence identified in the figure. In some aspects, a nucleic acid construct comprising a sequence set forth as SEQ ID NO: 20 is provided.

(SEQ ID NO: 20)
AGCTCATTAATACGACTCACTATACAAAACAAACGAATCTCAAGCAATCAAGCATTCTACTTCTATTGCAGC
AATTTAAATCATTTCTTTTAAAGCAAAAGCAATTTTCTGAAAATTTTCACCATTTACGAACGATAACGCCAC
CATGCGCCTGTCCAAGACCCTGGTGGACATGGACATGGCCGACTACTCCGCCGCCCTGGACCCCGCCTACAC
CACCCTGGAGTTCGAGAACGTGCAGGTGCTGACCATGGGCAACGACACCTCCCCCTCCGAGGGCACCAACCT
GAACGCCCCCAACTCCCTGGGCGTGTCCGCCCTGTGCGCCATCTGCGGCGACCGCGCCACCGGCAAGCACTA
CGGCGCCTCCTCCTGCGACGGCTGCAAGGGCTTCTTCCGCCGCTCCGTGCGCAAGAACCACATGTACTCCTG
CCGCTTCTCCCGCCAGTGCGTGGTGGACAAGGACAAGCGCAACCAGTGCCGCTACTGCCGCCTGAAGAAGTG
CTTCCGCGCCGGCATGAAGAAGGAGGCCGTGCAGAACGAGCGCGACCGCATCTCCACCCGCCGCTCCTCCTA
CGAGGACTCCTCCCTGCCCTCCATCAACGCCCTGCTGCAGGCCGAGGTGCTGTCCCGCCAGATCACCTCCCC
CGTGTCCGGCATCAACGGCGACATCCGCGCCAAGAAGATCGCCTCCATCGCCGACGTGTGCGAGTCCATGAA
GGAGCAGCTGCTGGTGCTGGTGGAGTGGGCCAAGTACATCCCCGCCTTCTGCGAGCTGCCCCTGGACGACCA
GGTGGCCCTGCTGCGCGCCCACGCCGGCGAGCACCTGCTGCTGGGCGCCACCAAGCGCTCCATGGTGTTCAA
GGACGTGCTGCTGCTGGGCAACGACTACATCGTGCCCCGCCACTGCCCCGAGCTGGCCGAGATGTCCCGCGT
GTCCATCCGCATCCTGGACGAGCTGGTGCTGCCCTTCCAGGAGCTGCAGATCGACGACAACGAGTACGCCTA
CCTGAAGGCCATCATCTTCTTCGACCCCGACGCCAAGGGCCTGTCCGACCCCGGCAAGATCAAGCGCCTGCG
CTCCCAGGTGCAGGTGTCCCTGGAGGACTACATCAACGACCGCCAGTACGACTCCCGCGGCCGCTTCGGCGA
GCTGCTGCTGCTGCTGCCCACCCTGCAGTCCATCACCTGGCAGATGATCGAGCAGATCCAGTTCATCAAGCT
GTTCGGCATGGCCAAGATCGACAACCTGCTGCAGGAGATGCTGCTGGGCGGCTCCCCCTCCGACGCCCCCCA
CGCCCACCACCCCCTGCACCCCCACCTGATGCAGGAGCACATGGGCACCAACGTGATCGTGGCCAACACCAT
GCCCACCCACCTGTCCAACGGCCAGATGTGCGAGTGGCCCCGCCCCCGCGGCCAGGCCGCCACCCCCGAGAC
CCCCCAGCCCTCCCCCCCCGGCGGCTCCGGCTCCGAGCCCTACAAGCTGCTGCCCGGCGCCGTGGCCACCAT
CGTGAAGCCCCTGTCCGCCATCCCCCAGCCCACCATCACCAAGCAGGAGGTGATCTAAACTAGTAGTGACTG
ACTAGGATCTGGTTACCACTAAACCAGCCTCAAGAACACCCGAATGGAGTCTCTAAGCTACATAATACCAAC
TTACACTTACAAAATGTTGTCCCCCAAAATGTAGCCATTCGTATCTGCTCCTAATAAAAAGAAAGTTTCTTC
ACATTCTAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAA

In some aspects, the DNA sequence of SEQ ID NO: 20 is used for the manufacture of therapeutic RNA construct encoding HNF4α2, for example, for use in the disclosed methods. In some aspects, a therapeutic RNA comprising the nucleotide sequence set forth as SEQ ID NO: 29, which corresponds to the DNA sequence of SEQ ID NO: 20, is provided.

(SEQ ID NO: 29)
AGCUCAUUAAUACGACUCACUAUACAAAACAAACGAAUCUCAAGCAAUCAAGCAUUCUACUUCUAUUGCAGC
AAUUUAAAUCAUUUCUUUUAAAGCAAAAGCAAUUUUCUGAAAAUUUUCACCAUUUACGAACGAUAACGCCAC
CAUGCGCCUGUCCAAGACCCUGGUGGACAUGGACAUGGCCGACUACUCCGCCGCCCUGGACCCCGCCUACAC
CACCCUGGAGUUCGAGAACGUGCAGGUGCUGACCAUGGGCAACGACACCUCCCCCUCCGAGGGCACCAACCU
GAACGCCCCCAACUCCCUGGGCGUGUCCGCCCUGUGCGCCAUCUGCGGCGACCGCGCCACCGGCAAGCACUA
CGGCGCCUCCUCCUGCGACGGCUGCAAGGGCUUCUUCCGCCGCUCCGUGCGCAAGAACCACAUGUACUCCUG
CCGCUUCUCCCGCCAGUGCGUGGUGGACAAGGACAAGCGCAACCAGUGCCGCUACUGCCGCCUGAAGAAGUG
CUUCCGCGCCGGCAUGAAGAAGGAGGCCGUGCAGAACGAGCGCGACCGCAUCUCCACCCGCCGCUCCUCCUA
CGAGGACUCCUCCCUGCCCUCCAUCAACGCCCUGCUGCAGGCCGAGGUGCUGUCCCGCCAGAUCACCUCCCC
CGUGUCCGGCAUCAACGGCGACAUCCGCGCCAAGAAGAUCGCCUCCAUCGCCGACGUGUGCGAGUCCAUGAA
GGAGCAGCUGCUGGUGCUGGUGGAGUGGGCCAAGUACAUCCCCGCCUUCUGCGAGCUGCCCCUGGACGACCA
GGUGGCCCUGCUGCGCGCCCACGCCGGCGAGCACCUGCUGCUGGGCGCCACCAAGCGCUCCAUGGUGUUCAA
GGACGUGCUGCUGCUGGGCAACGACUACAUCGUGCCCCGCCACUGCCCCGAGCUGGCCGAGAUGUCCCGCGU
GUCCAUCCGCAUCCUGGACGAGCUGGUGCUGCCCUUCCAGGAGCUGCAGAUCGACGACAACGAGUACGCCUA
CCUGAAGGCCAUCAUCUUCUUCGACCCCGACGCCAAGGGCCUGUCCGACCCCGGCAAGAUCAAGCGCCUGCG
CUCCCAGGUGCAGGUGUCCCUGGAGGACUACAUCAACGACCGCCAGUACGACUCCCGCGGCCGCUUCGGCGA
GCUGCUGCUGCUGCUGCCCACCCUGCAGUCCAUCACCUGGCAGAUGAUCGAGCAGAUCCAGUUCAUCAAGCU
GUUCGGCAUGGCCAAGAUCGACAACCUGCUGCAGGAGAUGCUGCUGGGCGGCUCCCCCUCCGACGCCCCCCA
CGCCCACCACCCCCUGCACCCCCACCUGAUGCAGGAGCACAUGGGCACCAACGUGAUCGUGGCCAACACCAU
GCCCACCCACCUGUCCAACGGCCAGAUGUGCGAGUGGCCCCGCCCCCGCGGCCAGGCCGCCACCCCCGAGAC
CCCCCAGCCCUCCCCCCCCGGCGGCUCCGGCUCCGAGCCCUACAAGCUGCUGCCCGGCGCCGUGGCCACCAU
CGUGAAGCCCCUGUCCGCCAUCCCCCAGCCCACCAUCACCAAGCAGGAGGUGAUCUAAACUAGUAGUGACUG
ACUAGGAUCUGGUUACCACUAAACCAGCCUCAAGAACACCCGAAUGGAGUCUCUAAGCUACAUAAUACCAAC
UUACACUUACAAAAUGUUGUCCCCCAAAAUGUAGCCAUUCGUAUCUGCUCCUAAUAAAAAGAAAGUUUCUUC
ACAUUCUAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAA

### HNF4α1

In other aspects, the presently disclosed methods utilize a recombinant mRNA encoding HNF4α1. In one aspect, an mRNA of use includes an in vitro-transcribed nucleic acid. The RNA is produced by in vitro transcription using a plasmid DNA template generated synthetically. Exemplary plasmids include, but are not limited to, those provided in Addgene—Plamid #178114, Addgene—Plasmid #127256, System Biosciences Inc, Cat #MR700A-1- or Cat #MR800A-1 (mRNAEXPRESS™ GFP Transcript plasmids). DNA of interest from any source can be directly converted by PCR into a template for in vitro mRNA synthesis using appropriate primers and RNA polymerase. In various aspects, plasmid is used to generate a template for in vitro transcription of mRNA which is used in the disclosed methods.

An exemplary amino acid sequence of HNF4α1 is provided as:

(SEQ ID NO: 21)
MRLSKTLLVDMDMADYSAALDPAYTTLEFENVQVLTMGNDTSPSEGTNL
NAPNSLGVSALCAICGDRATGKHYGASSCDGCKGFFRRSVRKNHMYSCR
FSRQCVVDKDKRNQCRYCRLKKCFRAGMKKEAVQNERDRISTRRSSYED
SSLPSINALLQAEVLSRQITSPVSGINGDIRAKKIASIADVCESMKEQL
LVLVEWAKYIPAFCELPLDDQVALLRAHAGEHLLLGATKRSMVFKDVLL
LGNDYIVPRHCPELAEMSRVSIRILDELVLPFQELQIDDNEYAYLKAII
FFDPDAKGLSDPGKIKRLRSQVQVSLEDYINDRQYDSRGRFGELLLLLP
TLQSITWQMIEQIQFIKLFGMAKIDNLLQEMLLGGSPSDAPHAHHPLHP
HLMQEHMGTNVIVANTMPTHLSNGQMSTPETPQPSPPGGSGSEPYKLLP
GAVATIVKPLSAIPQPTITKQEVI

See also NCBI Reference Sequence: NM_178849.3, NP_849180.1 as available on Oct. 28, 2022, incorporated herein by reference.

In some aspects, an mRNA of use in the disclosed methods encodes a polypeptide at least 85%, 90%, 95%, 96%, 97%, 98% or 99% identical to SEQ ID NO: 21. In further aspects, an mRNA of use in the disclosed methods encodes a polypeptide set forth as SEQ ID NO: 21. In yet other aspects, an mRNA of use in the disclosed methods encodes a polypeptide with at most 1, 2, 3, 4, or 5 conservative substitutions in SEQ ID NO: 21.

An exemplary RNA encoding the HNF4α1 protein of SEQ ID NO: 21 is provided below:

(SEQ ID NO: 22)
AUGCGACUCUCCAAAACCCUCGUCGACAUGGACAUGGCCGACUACAGUG
CUGCACUGGACCCAGCCUACACCACCCUGGAAUUUGAGAAUGUGCAGGU
GUUGACGAUGGGCAAUGACACGUCCCCAUCAGAAGGCACCAACCUCAAC
GCGCCCAACAGCCUGGGUGUCAGCGCCCUGUGUGCCAUCUGCGGGGACC
GGGCCACGGGCAAACACUACGGUGCCUCGAGCUGUGACGGCUGCAAGGG
CUUCUUCCGGAGGAGCGUGCGGAAGAACCACAUGUACUCCUGCAGAUUU
AGCCGGCAGUGCGUGGUGGACAAAGACAAGAGGAACCAGUGCCGCUACU
GCAGGCUCAAGAAAUGCUUCCGGGCUGGCAUGAAGAAGGAAGCCGUCCA
GAAUGAGCGGGACCGGAUCAGCACUCGAAGGUCAAGCUAUGAGGACAGC
AGCCUGCCCUCCAUCAAUGCGCUCCUGCAGGCGGAGGUCCUGUCCCGAC
AGAUCACCUCCCCCGUCUCCGGGAUCAACGGCGACAUUCGGGCGAAGAA
GAUUGCCAGCAUCGCAGAUGUGUGUGAGUCCAUGAAGGAGCAGCUGCUG
GUUCUCGUUGAGUGGGCCAAGUACAUCCCAGCUUUCUGCGAGCUCCCCC
UGGACGACCAGGUGGCCCUGCUCAGAGCCCAUGCUGGCGAGCACCUGCU
GCUCGGAGCCACCAAGAGAUCCAUGGUGUUCAAGGACGUGCUGCUCCUA
GGCAAUGACUACAUUGUCCCUCGGCACUGCCCGGAGCUGGCGGAGAUGA
GCCGGGUGUCCAUACGCAUCCUUGACGAGCUGGUGCUGCCCUUCCAGGA
GCUGCAGAUCGAUGACAAUGAGUAUGCCUACCUCAAAGCCAUCAUCUUC
UUUGACCCAGAUGCCAAGGGGCUGAGCGAUCCAGGGAAGAUCAAGCGGC
UGCGUUCCCAGGUGCAGGUGAGCUUGGAGGACUACAUCAACGACCGCCA
GUAUGACUCGCGUGGCCGCUUUGGAGAGCUGCUGCUGCUGCUGCCCACC
UUGCAGAGCAUCACCUGGCAGAUGAUCGAGCAGAUCCAGUUCAUCAAGC
UCUUCGGCAUGGCCAAGAUUGACAACCUGUUGCAGGAGAUGCUGCUGGG
AGGGUCCCCCAGCGAUGCACCCCAUGCCCACCACCCCCUGCACCCUCAC
CUGAUGCAGGAACAUAUGGGAACCAACGUCAUCGUUGCCAACACAAUGC
CCACUCACCUCAGCAACGGACAGAUGUCCACCCCUGAGACCCCACAGCC
CUCACCGCCAGGUGGCUCAGGGUCUGAGCCCUAUAAGCUCCUGCCGGGA
GCCGUCGCCACAAUCGUCAAGCCCCUCUCUGCCAUCCCCCAGCCGACCA
UCACCAAGCAGGAAGUUAUCUAG

An exemplary cDNA sequence encoding the HNF4α1 protein of SEQ ID NO: 21 and corresponding to the mRNA sequence of SEQ ID NO: 22 is provided below (also see FIG. 21A):

(SEQ ID NO: 26)
ATGCGACTCTCCAAAACCCTCGTCGACATGGACATGGCCGACTACAGTG
CTGCACTGGACCCAGCCTACACCACCCTGGAATTTGAGAATGTGCAGGT
GTTGACGATGGGCAATGACACGTCCCCATCAGAAGGCACCAACCTCAAC
GCGCCCAACAGCCTGGGTGTCAGCGCCCTGTGTGCCATCTGCGGGGACC
GGGCCACGGGCAAACACTACGGTGCCTCGAGCTGTGACGGCTGCAAGGG
CTTCTTCCGGAGGAGCGTGCGGAAGAACCACATGTACTCCTGCAGATTT
AGCCGGCAGTGCGTGGTGGACAAAGACAAGAGGAACCAGTGCCGCTACT
GCAGGCTCAAGAAATGCTTCCGGGCTGGCATGAAGAAGGAAGCCGTCCA
GAATGAGCGGGACCGGATCAGCACTCGAAGGTCAAGCTATGAGGACAGC
AGCCTGCCCTCCATCAATGCGCTCCTGCAGGCGGAGGTCCTGTCCCGAC
AGATCACCTCCCCCGTCTCCGGGATCAACGGCGACATTCGGGCGAAGAA
GATTGCCAGCATCGCAGATGTGTGTGAGTCCATGAAGGAGCAGCTGCTG
GTTCTCGTTGAGTGGGCCAAGTACATCCCAGCTTTCTGCGAGCTCCCCC
TGGACGACCAGGTGGCCCTGCTCAGAGCCCATGCTGGCGAGCACCTGCT
GCTCGGAGCCACCAAGAGATCCATGGTGTTCAAGGACGTGCTGCTCCTA
GGCAATGACTACATTGTCCCTCGGCACTGCCCGGAGCTGGCGGAGATGA
GCCGGGTGTCCATACGCATCCTTGACGAGCTGGTGCTGCCCTTCCAGGA
GCTGCAGATCGATGACAATGAGTATGCCTACCTCAAAGCCATCATCTTC
TTTGACCCAGATGCCAAGGGGCTGAGCGATCCAGGGAAGATCAAGCGGC
TGCGTTCCCAGGTGCAGGTGAGCTTGGAGGACTACATCAACGACCGCCA
GTATGACTCGCGTGGCCGCTTTGGAGAGCTGCTGCTGCTGCTGCCCACC
TTGCAGAGCATCACCTGGCAGATGATCGAGCAGATCCAGTTCATCAAGC
TCTTCGGCATGGCCAAGATTGACAACCTGTTGCAGGAGATGCTGCTGGG
AGGGTCCCCCAGCGATGCACCCCATGCCCACCACCCCCTGCACCCTCAC
CTGATGCAGGAACATATGGGAACCAACGTCATCGTTGCCAACACAATGC
CCACTCACCTCAGCAACGGACAGATGTCCACCCCTGAGACCCCACAGCC
CTCACCGCCAGGTGGCTCAGGGTCTGAGCCCTATAAGCTCCTGCCGGGA
GCCGTCGCCACAATCGTCAAGCCCCTCTCTGCCATCCCCCAGCCGACCA
TCACCAAGCAGGAAGTTATCTAG

In some aspects, the cDNA sequence of SEQ ID NO: 26 is used for the manufacture of RNA construct encoding SEQ ID NO: 1.

A codon-optimized mRNA encoding the HNF4α1 protein of SEQ ID NO: 21 is provided as SEQ ID NO: 30:

(SEQ ID NO: 30)
AUGCGCCUGUCCAAGACCCUGGUGGACAUGGACAUGGCCGACUACUCCG
CCGCCCUGGACCCCGCCUACACCACCCUGGAGUUCGAGAACGUGCAGGU
GCUGACCAUGGGCAACGACACCUCCCCCUCCGAGGGCACCAACCUGAAC
GCCCCCAACUCCCUGGGCGUGUCCGCCCUGUGCGCCAUCUGCGGCGACC
GCGCCACCGGCAAGCACUACGGCGCCUCCUCCUGCGACGGCUGCAAGGG
CUUCUUCCGCCGCUCCGUGCGCAAGAACCACAUGUACUCCUGCCGCUUC
UCCCGCCAGUGCGUGGUGGACAAGGACAAGCGCAACCAGUGCCGCUACU
GCCGCCUGAAGAAGUGCUUCCGCGCCGGCAUGAAGAAGGAGGCCGUGCA
GAACGAGCGCGACCGCAUCUCCACCCGCCGCUCCUCCUACGAGGACUCC
UCCCUGCCCUCCAUCAACGCCCUGCUGCAGGCCGAGGUGCUGUCCCGCC
AGAUCACCUCCCCCGUGUCCGGCAUCAACGGCGACAUCCGCGCCAAGAA
GAUCGCCUCCAUCGCCGACGUGUGCGAGUCCAUGAAGGAGCAGCUGCUG
GUGCUGGUGGAGUGGGCCAAGUACAUCCCCGCCUUCUGCGAGCUGCCCC
UGGACGACCAGGUGGCCCUGCUGCGCGCCCACGCCGGCGAGCACCUGCU
GCUGGGCGCCACCAAGCGCUCCAUGGUGUUCAAGGACGUGCUGCUGCUG
GGCAACGACUACAUCGUGCCCCGCCACUGCCCCGAGCUGGCCGAGAUGU
CCCGCGUGUCCAUCCGCAUCCUGGACGAGCUGGUGCUGCCCUUCCAGGA
GCUGCAGAUCGACGACAACGAGUACGCCUACCUGAAGGCCAUCAUCUUC
UUCGACCCCGACGCCAAGGGCCUGUCCGACCCCGGCAAGAUCAAGCGCC
UGCGCUCCCAGGUGCAGGUGUCCCUGGAGGACUACAUCAACGACCGCCA
GUACGACUCCCGCGGCCGCUUCGGCGAGCUGCUGCUGCUGCUGCCCACC
CUGCAGUCCAUCACCUGGCAGAUGAUCGAGCAGAUCCAGUUCAUCAAGC
UGUUCGGCAUGGCCAAGAUCGACAACCUGCUGCAGGAGAUGCUGCUGGG
CGGCUCCCCCUCCGACGCCCCCCACGCCCACCACCCCCUGCACCCCCAC
CUGAUGCAGGAGCACAUGGGCACCAACGUGAUCGUGGCCAACACCAUGC
CCACCCACCUGUCCAACGGCCAGAUGUCCACCCCCGAGACCCCCCAGCC
CUCCCCCCCCGGCGGCUCCGGCUCCGAGCCCUACAAGCUGCUGCCCGGC
GCCGUGGCCACCAUCGUGAAGCCCCUGUCCGCCAUCCCCCAGCCCACCA
UCACCAAGCAGGAGGUGAUC

In some aspects, an mRNA sequence encoding HNF4α1 is at least about 80% identical to SEQ ID NO: 22 or SEQ ID NO: 30. In other aspects, an mRNA encoding HNF4α1 is at least about 85%, 90%, 95%, 96%, 97%, 98% or 99% identical to SEQ ID NO: 22 or SEQ ID NO: 30. In further aspects, the mRNA encoding HNF4α1 is at least about 95%, 96%, 97%, 98% or 99% identical to SEQ ID NO: 22 or SEQ ID NO: 30.

A codon-optimized cDNA sequence encoding the HNF4α1 protein of SEQ ID NO: 21 and corresponding to the mRNA of SEQ ID NO: 30 is provided below:

(SEQ ID NO: 33)
ATGCGCCTGTCCAAGACCCTGGTGGACATGGACATGGCCGACTACTCCG
CCGCCCTGGACCCCGCCTACACCACCCTGGAGTTCGAGAACGTGCAGGT
GCTGACCATGGGCAACGACACCTCCCCCTCCGAGGGCACCAACCTGAAC
GCCCCCAACTCCCTGGGCGTGTCCGCCCTGTGCGCCATCTGCGGCGACC
GCGCCACCGGCAAGCACTACGGCGCCTCCTCCTGCGACGGCTGCAAGGG
CTTCTTCCGCCGCTCCGTGCGCAAGAACCACATGTACTCCTGCCGCTTC
TCCCGCCAGTGCGTGGTGGACAAGGACAAGCGCAACCAGTGCCGCTACT
GCCGCCTGAAGAAGTGCTTCCGCGCCGGCATGAAGAAGGAGGCCGTGCA
GAACGAGCGCGACCGCATCTCCACCCGCCGCTCCTCCTACGAGGACTCC
TCCCTGCCCTCCATCAACGCCCTGCTGCAGGCCGAGGTGCTGTCCCGCC
AGATCACCTCCCCCGTGTCCGGCATCAACGGCGACATCCGCGCCAAGAA
GATCGCCTCCATCGCCGACGTGTGCGAGTCCATGAAGGAGCAGCTGCTG
GTGCTGGTGGAGTGGGCCAAGTACATCCCCGCCTTCTGCGAGCTGCCCC
TGGACGACCAGGTGGCCCTGCTGCGCGCCCACGCCGGCGAGCACCTGCT
GCTGGGCGCCACCAAGCGCTCCATGGTGTTCAAGGACGTGCTGCTGCTG
GGCAACGACTACATCGTGCCCCGCCACTGCCCCGAGCTGGCCGAGATGT
CCCGCGTGTCCATCCGCATCCTGGACGAGCTGGTGCTGCCCTTCCAGGA
GCTGCAGATCGACGACAACGAGTACGCCTACCTGAAGGCCATCATCTTC
TTCGACCCCGACGCCAAGGGCCTGTCCGACCCCGGCAAGATCAAGCGCC
TGCGCTCCCAGGTGCAGGTGTCCCTGGAGGACTACATCAACGACCGCCA
GTACGACTCCCGCGGCCGCTTCGGCGAGCTGCTGCTGCTGCTGCCCACC
CTGCAGTCCATCACCTGGCAGATGATCGAGCAGATCCAGTTCATCAAGC
TGTTCGGCATGGCCAAGATCGACAACCTGCTGCAGGAGATGCTGCTGGG
CGGCTCCCCCTCCGACGCCCCCCACGCCCACCACCCCCTGCACCCCCAC
CTGATGCAGGAGCACATGGGCACCAACGTGATCGTGGCCAACACCATGC
CCACCCACCTGTCCAACGGCCAGATGTCCACCCCCGAGACCCCCCAGCC
CTCCCCCCCCGGCGGCTCCGGCTCCGAGCCCTACAAGCTGCTGCCCGGC
GCCGTGGCCACCATCGTGAAGCCCCTGTCCGCCATCCCCCAGCCCACCA
TCACCAAGCAGGAGGTGATC

In some aspects, an mRNA sequence encoding HNF4α1 is provided that has an equivalent cDNA sequence at least about 80% identical to SEQ ID NO: 33. In other aspects, an mRNA encoding HNF4α1 is provided that has an equivalent cDNA sequence is at least about 85%, 90%, 95%, 96%, 97%, 98% or 99% identical to SEQ ID NO: 33. In other aspects, an mRNA encoding HNF4α1 is provided that has an equivalent cDNA sequence at least about 85%, 90%, 95%, 96%, 97%, 98% or 99% identical to SEQ ID NO: 33. In some aspects, an mRNA sequence encoding HNF4α1 is at least about 80% identical to SEQ ID NO: 22 or SEQ ID NO: 30 and encodes SEQ ID NO: 21. In other aspects, an mRNA encoding HNF4α1 is at least about 85%, 90%, 95%, 96%, 97%, 98% or 99% identical to SEQ ID NO: 22 or SEQ ID NO: 30 and encodes SEQ ID NO: 21. In further aspects, the mRNA encoding HNF4α1 is at least about 95%, 96%, 97%, 98% or 99% identical to SEQ ID NO: 22 or SEQ ID NO: 30 and encodes SEQ ID NO: 21.

An exemplary DNA sequence (SEQ ID NO: 23) encoding HNF4α1 that can be converted to mRNA for use in the disclosed methods is shown below and in FIG. 15, with the segments of the sequence identified in the figure. In some aspects, a nucleic acid construct comprising a sequence set forth as SEQ ID NO: 23 is provided.

(SEQ ID NO: 23)
agctCATTAATACGACTCACTATACAAAACAAACGAATCTCAAGCAATC
AAGCATTCTACTTCTATTGCAGCAATTTAAATCATTTCTTTTAAAGCAA
AAGCAATTTTCTGAAAATTTTCACCATTTACGAACGATAGCGCTgccac
cATGCGCCTGTCCAAGACCCTGGTGGACATGGACATGGCCGACTACTCC
GCCGCCCTGGACCCCGCCTACACCACCCTGGAGTTCGAGAACGTGCAGG
TGCTGACCATGGGCAACGACACCTCCCCCTCCGAGGGCACCAACCTGAA
CGCCCCCAACTCCCTGGGCGTGTCCGCCCTGTGCGCCATCTGCGGCGAC
CGCGCCACCGGCAAGCACTACGGCGCCTCCTCCTGCGACGGCTGCAAGG
GCTTCTTCCGCCGCTCCGTGCGCAAGAACCACATGTACTCCTGCCGCTT
CTCCCGCCAGTGCGTGGTGGACAAGGACAAGCGCAACCAGTGCCGCTAC
TGCCGCCTGAAGAAGTGCTTCCGCGCCGGCATGAAGAAGGAGGCCGTGC
AGAACGAGCGCGACCGCATCTCCACCCGCCGCTCCTCCTACGAGGACTC
CTCCCTGCCCTCCATCAACGCCCTGCTGCAGGCCGAGGTGCTGTCCCGC
CAGATCACCTCCCCCGTGTCCGGCATCAACGGCGACATCCGCGCCAAGA
AGATCGCCTCCATCGCCGACGTGTGCGAGTCCATGAAGGAGCAGCTGCT
GGTGCTGGTGGAGTGGGCCAAGTACATCCCCGCCTTCTGCGAGCTGCCC
CTGGACGACCAGGTGGCCCTGCTGCGCGCCCACGCCGGCGAGCACCTGC
TGCTGGGCGCCACCAAGCGCTCCATGGTGTTCAAGGACGTGCTGCTGCT
GGGCAACGACTACATCGTGCCCCGCCACTGCCCCGAGCTGGCCGAGATG
TCCCGCGTGTCCATCCGCATCCTGGACGAGCTGGTGCTGCCCTTCCAGG
AGCTGCAGATCGACGACAACGAGTACGCCTACCTGAAGGCCATCATCTT
CTTCGACCCCGACGCCAAGGGCCTGTCCGACCCCGGCAAGATCAAGCGC
CTGCGCTCCCAGGTGCAGGTGTCCCTGGAGGACTACATCAACGACCGCC
AGTACGACTCCCGCGGCCGCTTCGGCGAGCTGCTGCTGCTGCTGCCCAC
CCTGCAGTCCATCACCTGGCAGATGATCGAGCAGATCCAGTTCATCAAG
CTGTTCGGCATGGCCAAGATCGACAACCTGCTGCAGGAGATGCTGCTGG
GCGGCTCCCCCTCCGACGCCCCCCACGCCCACCACCCCCTGCACCCCCA
CCTGATGCAGGAGCACATGGGCACCAACGTGATCGTGGCCAACACCATG
CCCACCCACCTGTCCAACGGCCAGATGTCCACCCCCGAGACCCCCCAGC
CCTCCCCCCCCGGCGGCTCCGGCTCCGAGCCCTACAAGCTGCTGCCCGG
CGCCGTGGCCACCATCGTGAAGCCCCTGTCCGCCATCCCCCAGCCCACC
ATCACCAAGCAGGAGGTGATCtaataaactagtAGTGACTGACTAGGAT
CTGGTTACCACTAAACCAGCCTCAAGAACACCCGAATGGAGTCTCTAAG
CTACATAATACCAACTTACACTTACAAAATGTTGTCCCCCAAAATGTAG
CCATTCGTATCTGCTCCTAATAAAAAGAAAGTTTCTTCACATTCTAAAA
AAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAA.

In some aspects, the DNA sequence of SEQ ID NO: 23 is used for the manufacture of therapeutic RNA encoding HNF4α1, for example, for use in the disclosed methods. In some aspects, a therapeutic RNA comprising the nucleotide sequence set forth as SEQ ID NO: 32 is provided, which corresponds to the DNA sequence of SEQ ID NO: 23, is provided.

(SEQ ID NO: 32)
AGCUCAUUAAUACGACUCACUAUACAAAACAAACGAAUCUCAAGCAAUC
AAGCAUUCUACUUCUAUUGCAGCAAUUUAAAUCAUUUCUUUUAAAGCAA
AAGCAAUUUUCUGAAAAUUUUCACCAUUUACGAACGAUAGCGCUGCCAC
CAUGCGCCUGUCCAAGACCCUGGUGGACAUGGACAUGGCCGACUACUCC
GCCGCCCUGGACCCCGCCUACACCACCCUGGAGUUCGAGAACGUGCAGG
UGCUGACCAUGGGCAACGACACCUCCCCCUCCGAGGGCACCAACCUGAA
CGCCCCCAACUCCCUGGGCGUGUCCGCCCUGUGCGCCAUCUGCGGCGAC
CGCGCCACCGGCAAGCACUACGGCGCCUCCUCCUGCGACGGCUGCAAGG
GCUUCUUCCGCCGCUCCGUGCGCAAGAACCACAUGUACUCCUGCCGCUU
CUCCCGCCAGUGCGUGGUGGACAAGGACAAGCGCAACCAGUGCCGCUAC
UGCCGCCUGAAGAAGUGCUUCCGCGCCGGCAUGAAGAAGGAGGCCGUGC
AGAACGAGCGCGACCGCAUCUCCACCCGCCGCUCCUCCUACGAGGACUC
CUCCCUGCCCUCCAUCAACGCCCUGCUGCAGGCCGAGGUGCUGUCCCGC
CAGAUCACCUCCCCCGUGUCCGGCAUCAACGGCGACAUCCGCGCCAAGA
AGAUCGCCUCCAUCGCCGACGUGUGCGAGUCCAUGAAGGAGCAGCUGCU
GGUGCUGGUGGAGUGGGCCAAGUACAUCCCCGCCUUCUGCGAGCUGCCC
CUGGACGACCAGGUGGCCCUGCUGCGCGCCCACGCCGGCGAGCACCUGC
UGCUGGGCGCCACCAAGCGCUCCAUGGUGUUCAAGGACGUGCUGCUGCU
GGGCAACGACUACAUCGUGCCCCGCCACUGCCCCGAGCUGGCCGAGAUG
UCCCGCGUGUCCAUCCGCAUCCUGGACGAGCUGGUGCUGCCCUUCCAGG
AGCUGCAGAUCGACGACAACGAGUACGCCUACCUGAAGGCCAUCAUCUU
CUUCGACCCCGACGCCAAGGGCCUGUCCGACCCCGGCAAGAUCAAGCGC
CUGCGCUCCCAGGUGCAGGUGUCCCUGGAGGACUACAUCAACGACCGCC
AGUACGACUCCCGCGGCCGCUUCGGCGAGCUGCUGCUGCUGCUGCCCAC
CCUGCAGUCCAUCACCUGGCAGAUGAUCGAGCAGAUCCAGUUCAUCAAG
CUGUUCGGCAUGGCCAAGAUCGACAACCUGCUGCAGGAGAUGCUGCUGG
GCGGCUCCCCCUCCGACGCCCCCCACGCCCACCACCCCCUGCACCCCCA
CCUGAUGCAGGAGCACAUGGGCACCAACGUGAUCGUGGCCAACACCAUG
CCCACCCACCUGUCCAACGGCCAGAUGUCCACCCCCGAGACCCCCCAGC
CCUCCCCCCCCGGCGGCUCCGGCUCCGAGCCCUACAAGCUGCUGCCCGG
CGCCGUGGCCACCAUCGUGAAGCCCCUGUCCGCCAUCCCCCAGCCCACC
AUCACCAAGCAGGAGGUGAUCUAAUAAACUAGUAGUGACUGACUAGGAU
CUGGUUACCACUAAACCAGCCUCAAGAACACCCGAAUGGAGUCUCUAAG
CUACAUAAUACCAACUUACACUUACAAAAUGUUGUCCCCCAAAAUGUAG
CCAUUCGUAUCUGCUCCUAAUAAAAAGAAAGUUUCUUCACAUUCUAAAA
AAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAA

### Additional Regulatory Elements

In some aspects, with regarding to the encoded protein, a signal peptide is linked to HNF4α2 or HNF4α1. The sequence shown in FIG. 14A encodes the native HNF4α2 signal peptide or HNF4α1 signal peptide, respectively

The HNF4α2 signal peptide or HNF4α1 signal peptide can be replaced with a heterologous signal peptide. In some aspects, the mRNA encodes the native signal peptide and does not encode a heterologous signal peptide. In other aspects, the mRNA encodes a heterologous signal peptide and HNF4α2 (without the native signal peptide). In other aspects, the mRNA encodes a heterologous signal peptide and HNF4α1 (without the native signal peptide).

The heterologous signal peptide contains the following regions, for example:

| n-region | h-region | cleavage site |
|---|---|---|
| positively charged | hydophobic | signal peptidase |

In some aspects, a nucleic acid sequence encoding signal peptide can be 5′ to the nucleic acid sequence encoding HNF4α2 or HNF4α1. In other aspects, a nucleic acid sequence encoding signal peptide can be 3′ to the nucleic acid sequence encoding HNF4α2 or HNF4α1. Thus, in the encoded protein, a heterologous signal peptide can be 5′ or 3′ to HNF4α2 or HNF4α1. In one aspect, the signal peptide comprises or consists of one of:

| | | SEQ |
|---|---|---|
| Name | Sequence | ID NO: |
| L1 | MDMRVPAQLLGLLLLWLSGARC | 3 |
| H7 | MEFGLSWVFLVALFRGVQC | 4 |
| Igkappa | METDTLLLWVLLLWVPGSTG | 5 |
| Mod HAS | MKWVTFISLLFLFSSSSRA | 6 |
| Optimized L2 | MRMQLLLLIALSLALVTNS | 7 |

In some aspects, the mRNA has 5′ and 3′ UTRs. In one aspect, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, the 5′ and 3′ UTR lengths can be modified as needed to increase translation efficiency following transfection of the transcribed RNA

The 5′ and 3′ UTRs can be the naturally occurring, endogenous 5′ and 3′ UTRs for the gene encoding HNF4α2. The 5′ and 3′ UTRs can be the naturally occurring, endogenous 5′ and 3′ UTRs for the gene encoding HNF4α1. Alternatively, UTR sequences that are not endogenous to the gene of interest can be added by incorporating the UTR sequences into the forward and reverse primers or by any other modifications of the template. Without being bound by theory, the use of UTR sequences that are not endogenous to the gene of interest can be useful for modifying the stability and/or translation efficiency of the RNA. Without being bound by theory, AU-rich elements in 3′ UTR sequences can decrease the stability of mRNA. Therefore, 3′ UTRs can be selected or designed to increase the stability of the transcribed RNA based on properties of UTRs that are well known in the art.

In one aspect, the 5′ UTR can contain the Kozak sequence of the endogenous gene. Alternatively, when a 5′ UTR that is not endogenous to the gene of interest is used, a consensus Kozak sequence can be designed by adding the 5′ UTR sequence. Kozak sequences can increase the efficiency of translation of some RNA transcripts, but are not required for all RNAs to enable efficient translation.

Thus, in some aspects, the mRNAs that encode an HNF-4a isoform include a 5′ UTR and/or a 3′ UTR that results in greater mRNA stability and higher expression of the mRNA in the cells.

In some embodiments, the mRNA includes a Kozak sequence in the 5′ UTR. The Kozak sequence can be, for example, ACCAUGG or GCCACCAUGC (SEQ ID NO: 31), which include the start codon for HNF-4a isoform. This Kozak sequence can be included in any of the 5′ UTRs listed herein.

An exemplary 5′ UTR comprises, or consists of:

(SEQ ID NO: 8)
UCUCAACACAACAUAUACAAAACAAACGAAUCUCAAGCAAUCAAGCA
UUCUACUUCUAUUGCAGCAAUUUAAAUcauuucuuuuaaagcaaaag
caauuuucugaaaauuuucaccauuuacgaacgau.

In other aspects, the 5′ UTR comprises, or consists of

(SEQ ID NO: 9)
CGAACUAGUAUUCUUCUGGUCCCCACAGACUCAGAGAGAACCCGCCA
CC

In further aspects, the 5′UTR comprises, or consists of:

(SEQ ID NO: 10)
AGGAGGGUUUUUACC.

In more aspects, the 5′ UTR comprises or consists of:

(SEQ ID NO: 24)
CAAAACAAACGAAUCUCAAGCAAUCAAGCAUUCUACUUCUAUUGCAG
CAAUUUAAAUCAUUUCUUUUAAAGCAAAAGCAAUUUUCUGAAAAUUU
UCACCAUUUACGAACGAU

In yet other aspects, the 3′ UTR comprises or consists of:

(SEQ ID NO: 11)
AACCAGCCUCAAGAACACCCGAAUGGAGUCUCUAAGCUACAUAAUAC
CAACUUACACUUACAAAAUGUUGUCCCCCAAAAUGUAGCCAUUCGUA
UCUGCUCCUAAUAAAAAGAAAGUUUCUU.

In some aspects, additional sequence, such as plasmid sequences, can be included. Additional 3′UTR and 5″ UTR sequences are shown in FIGS. 14A, 15 and 16.

In further aspects, the 3′ UTR comprises, or consists of one of the following mRNA sequences:

Beta-globin
(SEQ ID NO: 12)
AGUGACUGACUAGGAUCUGGUUACCACUAAACCAGCCUCAAGAACAC
CCGAAUGGAGUCUCUAAGCUACAUAAUACCAACUUACACUUACAAAA
UGUUGUCCCCCAAAAUGUAGCCAUUCGUAUCUGCUCCUAAUAAAAAG
AAAGUUUCUUCACAUUCU
Alpha-globin
(SEQ ID NO: 13)
UAAGCUGGAGCCUCGGUGGCCAUGCUUCUUGCCCCUUGGGCCUCCCC
CCAGCCCCUCCUCCCCUUCCUGCACCCGUACCCCCGUGGUCUUUGAA
UAAAGUCUGAGUGGGCGGC
Beta-globin Containing an inverted repeat
(SEQ ID NO: 14)
UUCUAGAGGAUCCAGAUCUUAAGUAAGUAAGCUCGAGAGCUCGCUUU
CUUGCUGUCCAAUUUCUAUUAAAGGUUCCUUUGUUCCCUAAGUCCAA
CUACUAAACUGGGGGAUAUUAUGAAGGGCCUUGAGCAUCUGGAUUCU
GCCUAAUAAAAAACAUUUAUUUUCAUUGCUGCGUCGAGAGCUCGCUU
UCUUGCUGUCCAAUUUCUAUUAAAGGUUCCUUUGUUCCCUAAGUCCA
ACUACUAAACUGGGGGAUAUUAUGAAGGGCCUUGAGCAUCUGGAUUC
UGCCUAAUAAAAAACAUUUAUUUUCAUUGCUGCGUCGAGAGCU
Satellite tobacco necrosis virus 1
(SEQ ID NO: 15)
UCCCAGAGGUUCACAAUGUUAGUGAUGGGCGCUGAAAGAUGCGUAGC
UACCCUUCUGGAGCCACUUCCUGGUGGUAAGCAGAAAUCCAAGGGUA
CGGUGGUACGGUGGAAAGCAGUCCCAGCUCUGCAUUGGGAAC
Preprolactin
(SEQ ID NO: 16)
AACAACAACUGCUAACGAGCUCGUAAGCCCACAUUCCAUCCUUUCCA
UUUCUGAGAUGGUUCUUAAUGAUCCAUUCCCUGGCAAACUUCUCUGA
GCUUUAUAGCUUUGUAAUGCAUGCCUUGGCUCUAAUGGGUUUCAUCU
U
Triple helix-poly(A)
(SEQ ID NO: 17)
UUCUAGAGGAUCCAGAUCUUAAGUAAGUAAGCUCGAGAGCUCGCUUU
CUUGCUGUCCAAUUUCUAUUAAAGGUUCCUUUGUUCCCUAAGUCCAA
CUACUAAACUGGGGGAUAUUAUGAAGGGCCUUGAGCAUCUGGAUUCU
GCCUAAUAAAAAACAUUUAUUUUCAUUGCUGCGUCGAGAGCUCGCUU
UCUUGCUGUCCAAUUUCUAUUAAAGGUUCCUUUGUUCCCUAAGUCCA
ACUACUAAACUGGGGGAUAUUAUGAAGGGCCUUGAGCAUCUGGAUUC
UGCCUAAUAAAAAACAUUUAUUUUCAUUGCUGCGUCGAGAGCU

In further aspects, the 3′ UTR comprises, or consists of:

(SEQ ID NO: 25)
AACCAGCCUCAAGAACACCCGAAUGGAGUCUCUAAGCUACAUAAUAC
CAACUUACACUUACAAAAUGUUGUCCCCCAAAAUGUAGCCAUUCGUA
UCUGCUCCUAAUAAAAAGAAAGUUUCUU

In some aspects, the 5′ UTR includes, or consists of SEQ ID NO: 24, and the 3′ UTR includes, or consists of, SEQ ID NO: 25.

In some aspects, the mRNA is polyadenylated. In some aspects, the mRNA comprises a poly-A tail (e.g., a poly-A tail having 50-200 nucleotides, such as 100-200, 150-200 nucleotides, or greater than 100 nucleotides), although in some aspects, a longer or a shorter poly-A tail is used. In some aspects, the poly A tail is 100, 101, 102, 103, 104, 105, 106, 107, 108, 109, or 110 nucleotides in length.

The recombinant mRNA encoding HNF4α2 or HNF4α1 can include a 5′ capping structure. 5′-capping of modified RNA can be completed concomitantly during IVT using the following chemical RNA cap analogs to generate the 5′-guanosine cap structure: 3′-O-Me-m7G(5′)ppp(5′)G; G(5′)ppp(5′)A; G(5′)ppp(5′)G; m7G(5′)ppp(5′)A; m7G(5′)ppp(5′)G (New England BioLabs, Ipswich, Mass.). In some aspects, 5′-capping of modified RNA may be completed post-transcriptionally using a Vaccinia Vims Capping Enzyme to generate the “Cap 0” structure: m7G(5′)ppp(5′)G (New England BioLabs, Ipswich, Mass.). Cap 1 structure can be generated using both Vaccinia ViJ.us Capping Enzyme and a 2′-0 methyl-transferase to generate: m7G(5′)ppp(5′)G-2′-O-methyl. Cap 2 structure can be generated from the Cap 1 structure followed by the 2′-O-methylation of the 5′-antepenultimate nucleotide using a 2′-0 methyl-transferase. Cap 3 structure can be generated from the Cap 2 structure followed by the 2′-O-methylation of the 5′-preantepenultimate nucleotide using a 2′-0 methyl-transferase. See U.S. Pat. No. 9,701,965, incorporated herein by reference.

To enable synthesis of RNA from a DNA template without the need for gene cloning, a promoter of transcription can be attached to the DNA template, upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one aspect, the promoter is a T7 RNA polymerase promoter, as described in U.S. Published Patent Application No. 2016/0030527A1, incorporated herein by reference. Other useful promoters include, but are not limited to, T3 and SP6 RNA polymerase promoters. Consensus nucleotide sequences for T7, T3 and SP6 promoters are known in the art.

The mRNA can be prepared using in vitro transcription (IVT). The IVT can be performed using any RNA polymerase as long as synthesis of the mRNA from the DNA template that encodes the RNA is specifically and sufficiently initiated from a respective cognate RNA polymerase promoter and full-length mRNA is obtained. In some preferred aspects, the RNA polymerase is selected from among T7 RNA polymerase, SP6 RNA polymerase and T3 RNA polymerase. In some other aspects, capped RNA is synthesized co-transcriptionally by using a dinucleotide cap analog in the IVT reaction (e.g., using an AMPLICAPT^{M} T7 Kit or a MESSAGEMAXT^{M} T7 ARCA-CAPPED MESSAGE Transcription Kit; EPICENTRE or CellScript, Madison, Wis., USA). If capping is performed co-transcriptionally, the dinucleotide cap analog can be an anti-reverse cap analog (ARCA). However, use of a separate IVT reaction, followed by capping with a capping enzyme system, which results in approximately 100% of the RNA being capped. Another option is co-transcriptional capping, which typically results in only about 80% of the RNA being capped. Thus, in some aspects, a high percentage of the mRNA molecules are capped (e.g., greater than 80%, greater than 90%, greater than 95%, greater than 98%, greater than 99%, greater than 99.5%, or greater than 99.9% of the population of mRNA molecules are capped).

In more aspects, the mRNA can be prepared by polyadenylation of an in vitro-transcribed (IVT) RNA using a poly(A) polymerase (e.g., yeast RNA polymerase or E. coli poly(A) polymerase). In some aspects, the mRNA is polyadenylated during in vitro transcription (IVT) by using a DNA template that encodes the poly(A) tail.

Poly(A) tails of RNAs can be further extended following in vitro transcription with the use of a poly(A) polymerase, such as E. coli polyA polymerase (E-PAP) or yeast polyA polymerase. In one aspect, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase.

An exemplary sequence encoding an mRNA of use in the disclosed methods is provided in FIG. 14A, with a schematic diagram of the domains provided in FIG. 14B. This sequence includes, in 5′ to 3′ order, a Cap, a 5′UTR including a Kozak sequence, a codon optimized sequence encoding HNF4α2, and a poly A tail. In this aspect, the RNA encodes the native signal peptide of HNF4α2. Another exemplary sequence encoding an mRNA of use in the disclosed methods is provided in FIG. 16. This sequence includes, in 5′ to 3′ order, a Cap, a 5′UTR including a Kozak sequence, a codon optimized sequence encoding HNF4α2, and a poly A tail. Another exemplary sequence encoding an mRNA of use in the disclosed methods is provided in FIG. 15. This sequence includes, in 5′ to 3′ order, a Cap, a 5′UTR including a Kozak sequence, a codon optimized sequence encoding HNF4α1, and a poly A tail.

In some aspects, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003). The conventional method of integration of polyA/T stretches into a DNA template is molecular cloning. If a polyA/T sequence integrated into plasmid DNA can cause plasmid instability in some cells, then this instability can be ameliorated through the use of recombination incompetent bacterial cells for plasmid propagation.

### VI. Modified Nucleic Acids

This disclosure is directed to the use of a recombinant mRNA encoding HNF4α2, including RNAs that contain one or more modified nucleosides (termed “modified nucleic acids”), which have useful properties including the lack of a substantial induction of the innate immune response of a cell into which the mRNA is introduced. This disclosure also is directed to the use of a recombinant mRNA encoding HNF4α1, including RNAs that contain one or more modified nucleosides (termed “modified nucleic acids”), which have useful properties including the lack of a substantial induction of the innate immune response of a cell into which the mRNA is introduced. As disclosed in U.S. Pat. No. 9,701,965, incorporated herein by reference, these modified nucleic acids enhance the efficiency of protein production, intracellular retention of nucleic acids, and viability of contacted cells, as well as possess reduced immunogenicity.

Provided are modified nucleic acids, such as a recombinant mRNA encoding HNF4α2, and including one, two, or more than two different nucleoside modifications. Also provided are modified nucleic acids, such as a recombinant mRNA encoding HNF4α1, and including one, two, or more than two different nucleoside modifications. In some aspects, the modified nucleic acid exhibits reduced degradation in a cell into which the nucleic acid is introduced, relative to a corresponding unmodified nucleic acid. For example, the degradation rate of the modified nucleic acid is reduced by about 10%, 20%, 30%, 40%, 50%, 60%, 70%, 80%, 90%, or greater than 90%, compared to the degradation rate of the corresponding unmodified nucleic acid. These nucleic acids do not substantially induce an innate immune response of a cell into which the mRNA is introduced.

In some aspects, modified nucleosides include pyridin-4-one ribonucleoside, 5-aza-uridine, 2-thio-5-aza-uridine, 2-thiomidine, 4-thio-pseudomidine, 2-thio-pseudowidine, 5-hydroxyuridine, 3-methylmidine, 5-carboxymethyl-uridine, 1-carboxymethyl-pseudoutidine, 5-propynyl-uridine, 1-propynyl-pseudomidine, 5-taurinomethyluridine, 1-taurinomethyl-pseudouridine, 5-taw.inomethyl-2-thio-utidine, 1-taurinomethyl-4-thio-uridine, 5-methyl-uridine, 1-methyl-pseudouridine, 4-thio-1-methyl-pseudouridine, 2-thio-1-methyl-pseudoutidine, 1-methyl-1-deaza-pseudomidine, 2-thio-1-methyl-1-deaza-pseudouridine, dihydrouridine, dihydropseudouridine, 2-thio-dihydromidine, 2-thio-dihydropseudoulidine, 2-methoxyuridine, 2-methoxy-4-thio-uridine, 4-methoxy-pseudomidine, and 4-methoxy-2-thio-pseudouridine.

In some aspects, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebulruine, 5-methyl-zebularine, 5-aza-2-thio-zebulru.ine, 2-thio-zebulaiine, 2-methoxy-cytidine, 2-methoxy-5-methyl-cytidine, 4-methoxy-pseudoisocytidine, and 4-methoxy-1-methyl-pseudoisocytidine.

In other aspects, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In specific aspects, a modified nucleoside is 5′-O-(1-Thiophosphate)-Adenosine, 5′-O-(1-Thiophosphate)-Cytidine, 5′-O-(1-thiophosphate)-Guanosine, 5′-O-(1-Thiophophate)-Uridine or 5′-O-(1-Thiophosphate)-Pseudouridine.

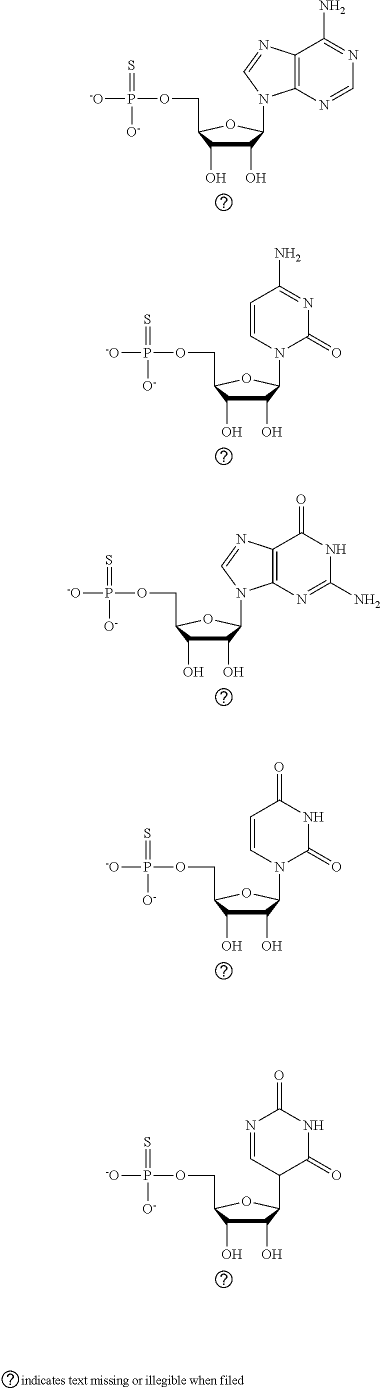

As disclosed in U.S. Pat. No. 9,701,965, incorporated herein by reference, the α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked nucleic acids are expected to also reduce the innate immune response through weaker binding/activation of cellular innate immune molecules.

In other aspects, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, J-methyl-6-thio-guanosine, N2-methyl-6-thio-guanosine, and N2,N2-dimethyl-6-thio-guanosine.

The disclosed mRNA can include a modified uridine or 1-methylpseudouridine. mRNA that contain either uridine, or 1-methylpseudouridine in place of uridine, the 1-methylpseudouridine-containing mRNA was translated at a higher level or for a longer duration than the mRNA that contained uridine. Therefore, in some aspects, one or more or all of the uridines contained in the mRNA(s) used in the methods disclosed herein is/are replaced by 1-methylpseudouridine (such as by substituting 1-methylpseudouridine-5′-triphosphate in an IVT reaction to synthesize the RNA in place of uridine-5′-triphosphate). However, in some aspects, the mRNA used in the disclosed methods contains uridine and does not contain 1-methylpseudouridine. In more aspects, the mRNA comprises at least one modified nucleoside (e.g., 1-methylpseudouridine (m1ψ), pseudouridine (Y), 5-methylcytosine (m⁵C), 5-methyluridine (m⁵U), 2′-O-methyluridine (Um or m^{2′-O}U), 2-thiouridine (s²U), or N⁶-methyladenosine (m⁶A)) in place of at least a portion of the corresponding unmodified canonical nucleoside (e.g., in place of substantially all of the corresponding unmodified A, C, G, or T canonical nucleoside). In some aspects, the mRNA comprises at least one modified nucleoside wherein the nucleotide is pseudouridine (ψ) or 5-methylcytosine (m⁵C). In some aspects, the mRNA comprises both pseudouridine (ψ) and 5-methylcytosine (m⁵C). In other aspects, the mRNA includes 1-methylpseudouridine.

In addition, in order to accomplish specific goals, a nucleic acid base, sugar moiety, or internucleotide linkage in one or more of the nucleotides of the mRNA that is introduced into a eukaryotic cell in any of the methods disclosed herein can comprise a modified nucleic acid base, sugar moiety, or internucleotide linkage.

The modified nucleic acids described herein are capable of evading an innate immune response of a cell into which the nucleic acids are introduced, thus increasing the efficiency of protein production in the cell. While it is advantageous to eliminate the innate immune response in a cell, the disclosure provides modified mRNAs that substantially reduce the immune response, including interferon signaling, without entirely eliminating such a response. In some aspects, the immune response is reduced by about 10%, 20%, 30%, 40%, 50%, 60%, 70%, 80%, 90%, 95%, 99%, 99.9%, or greater than 99.9%, as compared to the immune response induced by a corresponding unmodified nucleic acid. Such a reduction can be measured by expression or activity level of type 1 interferons or the expression of interferon-regulated genes such as the toll-like receptors (e.g., TLR7 and TLR8). Reduction of innate immune response can also be measured by decreased cell death following one or more administrations of modified RNAs to a cell population; e.g., cell death is about 10%, 25%, 50%, 75%, 85%, 90%, 95%, or over 95% less than the cell death frequency observed with a corresponding unmodified nucleic acid. Moreover, cell death may affect fewer than about 50%, 40%, 30%, 20%, 10%, 5%, 1%, 0.1%, 0.01%, or fewer than 0.01% of cells contacted with the modified nucleic acids.

Nucleic acids encoding for use in accordance with the disclosure may be prepared according to any available technique including, but not limited to chemical synthesis, enzymatic synthesis, which is generally termed in vitro transcription, enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

Modified nucleic acids need not be uniform1ψmodified along the entire length of the molecule. Different nucleotide modifications and/or backbone structures may exist at various positions in the nucleic acid. The nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least about 50% modified nucleotides, at least about 80% modified nucleotides, or at least about 90% modified nucleotides.

When transfected into mammalian cells, the modified mRNA can have a stability of between 12-18 hours or more than 18 hours, such as about 24, 36, 48, 60, 72 or greater than about 72 hours. In some aspects, the modified mRNA is stable for about 12 to about 72 hours, such as about 12 to about 48 hours, about 12 to about 36 hours, or about 12 to about 24 hours.

In a specific non-limiting example, the mRNA component is a modified mRNA with modified uridine, such as a 1-methylpseudouridine in place of uridine and a 7mG(5′)ppp(5′)N1mpNp cap.

### V. Lipid Nanoparticles

Lipid Nanoparticles are disclosed, for example, in PCT Publication No. 2021/150891, incorporated herein by reference. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/RNA compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

In an aspect, an RNA molecule is encapsulated in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171. 1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707.2005); and Zimmermann E et al., Electrolyte- and pH-stabilities of aqueous solid lipid nanoparticle (SLN) dispersions in artificial gastrointestinal media. Eur J Pharm Biopharm 52:203.2001). Methods are also disclosed in PCT Publication Nos. WO2021154763, US20210228707, WO2017070626 and US2019/0192646, which are incorporated by reference herein. See, also, Jackson et al., N Engl J Med., 383(20):1920-1921, 2020, incorporated by reference herein.

In several aspects, the mRNA is formulated in a lipid nanoparticle for administration to the subject; for example, comprising a PEG-modified lipid, a non-cationic lipid, a sterol, an ionizable lipid, or any combination thereof. In some aspects, the lipid nanoparticle is composed of 50 mol % ionizable lipid ((2 hydroxyethyl)(6 oxo 6-(undecycloxy)hexyl)amino)octanoate, 10 mol % 1,2 distearoyl sn glycerol-3 phosphocholine (DSPC), 38.5 mol % cholesterol, and 1.5 mol % 1-monomethoxypolyethyleneglycol-2,3,dimyristylglycerol with polyethylene glycol of average molecular weight 2000 (PEG2000 DMG). The mRNA/lipid nanoparticle composition may be provided in any suitable carrier, such as a sterile liquid for injection at a concentration of 0.5 mg/mL in 20 mM trometamol (Tris) buffer containing 87 mg/mL sucrose and 10.7 mM sodium acetate, at pH 7.5 and with appropriate diluent.

### VI. Pharmaceutical Compositions and Methods of Use

Pharmaceutical compositions including a recombinant mRNA encoding HNF4α2 are provided for use in treating end stage liver disease. Pharmaceutical compositions including a recombinant mRNA encoding HNF4α1 are provided for use in treating liver disease (such as liver cirrhosis or end-stage liver failure). In some aspects, the liver disease is a degenerative liver disease, such as non-alcoholic fatty liver disease (NASH) or alcohol related liver disease (ALD). In some aspects, the subject treated with the methods provided herein has liver disease with a Child-Pugh score level of A, B or C. The phmarmaceutical compositions can be formulated for local delivery to the liver. The compositions can be formulated and administered in a variety of ways (see, e.g., PCT Publication Nos. WO2021154763, US20210228707, WO2017070626 and US2019/0192646, which are incorporated by reference herein, which discloses pharmaceutical compositions as well as administration of such compositions and is incorporated herein by reference). The pharmaceutical compositions can include a lipid nanoparticle including the mRNA, as discussed above. The compositions can include a pharmaceutically acceptable carrier. In some aspects, the pharmacuetically acceptable carrier comprises a sterile buffer and a stabilizing agent.

Pharmaceutical compositions including a recombinant mRNA encoding HNF4α2 and/or a recombinant mRNA encoding HNF4α1 are provided that are formulated for local delivery to the liver. These pharmaceutical compostions can be delivered in vivo to the subject using any method suitable for local delivery to the liver, such as, but not limited to, intraperitoneal, intravenous, intramuscular, or intrahepatic (such as via hepatic vein or artery) administration.

Generally, it is desirable to prepare the compositions as pharmaceutical compositions appropriate for the intended application. Accordingly, methods for making a pharmaceutical composition containing the nucleic acid molecules, or vectors described above, are included herein. Typically, preparation of a pharmaceutical composition entails preparing a pharmaceutical composition that is essentially free of pyrogens, as well as any other impurities that could be harmful to humans or animals. Typically, the pharmaceutical composition contains appropriate salts and buffers to render the composition stable and allow for uptake of nucleic acids or virus by target cells.

Pharmaceutical compositions including nucleic acid molecules can be formulated for injection, such as for intrahepatic or intravenous administration. Such compositions are formulated generally by mixing a disclosed nucleic acid molecule at the desired degree of purity in a unit dosage injectable form (solution, suspension, or emulsion) with a pharmaceutically acceptable carrier, for example, one that is non-toxic to recipients at the dosages and concentrations employed and is compatible with other ingredients of the formulation. Pharmaceutical compositions can include an effective amount of the nucleic acid molecule dispersed (for example, dissolved or suspended) in a pharmaceutically acceptable carrier or excipient. Pharmaceutically acceptable carriers and/or pharmaceutically acceptable excipients are known in the art and are described, for example, in Remington's Pharmaceutical Sciences by E. W. Martin, Mack Publishing Co., Easton, PA, 19th Edition (1995). The nature of the carrier will depend on the particular mode of administration being employed. For example, formulations usually contain injectable fluids that include pharmaceutically and physiologically acceptable fluids, such as water, physiological saline, balanced salt solutions, aqueous dextrose, glycerol, or the like, as a vehicle. In addition, pharmaceutical compositions to be administered can contain minor amounts of non-toxic auxiliary substances, such as wetting or emulsifying agents, preservatives, pH buffering agents and the like, for example, sodium acetate or sorbitan monolaurate. A disclosed nucleic acid molecule can be suspended in an aqueous carrier, for example, in an isotonic or hypotonic buffer solution at a pH of about 3.0 to about 8.5, such as about 4.0 to about 8.0, about 6.5 to about 8.5, or about 7.4. Useful buffers include saline-buffered phosphate or an ionic boric acid buffer. The active ingredient, optionally together with excipients, can also be in the form of a lyophilisate and can be made into a solution prior to administration by the addition of suitable solvents.

The pharmaceutically acceptable carriers include any and all solvents, dispersion media, coatings, isotonic and absorption delaying agents, and the like. The use of such media and agents for pharmaceutically active substances is well-known in the art. Supplementary active ingredients also can be incorporated into the compositions. For example, certain pharmaceutical compositions can include the vectors or viruses in water, mixed with a suitable surfactant, such as hydroxy-propylcellulose. Dispersions also can be prepared in glycerol, liquid polyethylene glycols, and mixtures thereof as well as in oils. Under ordinary conditions of storage and use, these preparations contain a preservative to prevent the growth of microorganisms. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. In some aspects, the pharmaceutical composition comprises trometamol (Tris) buffer, sucrose, and sodium acetate, at physiological pH.

In some aspects, the excipients confer a protective effect to an mRNA, such that loss of mRNA, as well as transduceability resulting from formulation procedures, packaging, storage, transport, and the like, is minimized. These excipient compositions are therefore considered “nucleic acid-stabilizing” in the sense that they provide higher amounts of in that nucleic acid molecules than their non-protected counterparts, as measured using standard assays, see, for example, Published U.S. Application No. 2012/0219528, incorporated herein by reference. These compositions therefore demonstrate “enhanced transduceability levels” as compared to compositions lacking the particular excipients described herein and are therefore more stable than their non-protected counterparts.

Exemplary excipients that can used to protect a virion from activity degradative conditions include, but are not limited to, detergents, proteins, e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400, PEG-600, PEG-1000, PEG-1450, PEG-3350, PEG-6000, PEG-8000 and any molecular weights in between these values, with molecular weights of 1500 to 6000 preferred, propylene glycols (PG), sugar alcohols, such as a carbohydrate, preferably, sorbitol. The detergent, when present, can be an anionic, a cationic, a zwitterionic or a nonionic detergent. An exemplary detergent is a nonionic detergent. One suitable type of nonionic detergent is a sorbitan ester, e.g., polyoxyethylenesorbitan monolaurate (TWEEN®-20) polyoxyethylenesorbitan monopalmitate (TWEEN®-40), polyoxyethylenesorbitan monostearate (TWEEN®-60), polyoxyethylenesorbitan tristearate (TWEEN®-65), polyoxyethylenesorbitan monooleate (TWEEN®-80), polyoxyethylenesorbitan trioleate (TWEEN®-85), such as TWEEN®-20 and/or TWEEN®-80. These excipients are commercially available from a number of vendors, such as Sigma, St. Louis, Mo.

The amount of the various excipients in any of the disclosed compositions including the mRNA, varies and is readily determined by one of skill in the art. For example, a protein excipient, such as BSA, if present, will can be present at a concentration of between 1.0 weight (wt.) % to about 20 wt. %, such as 10 wt. %. If an amino acid such as glycine is used in the formulations, it can be present at a concentration of about 1 wt. % to about 5 wt. %. A carbohydrate, such as sorbitol, if present, can be present at a concentration of about 0.1 wt % to about 10 wt. %, such as between about 0.5 wt. % to about 15 wt. %, or about 1 wt. % to about 5 wt. %. If polyethylene glycol is present, it can generally be present on the order of about 2 wt. % to about 40 wt. %, such as about 10 wt. % top about 25 wt. %. If propylene glycol is used in the subject formulations, it will typically be present at a concentration of about 2 wt. % to about 60 wt. %, such as about 5 wt. % to about 30 wt. %. If a detergent such as a sorbitan ester (TWEEN®) is present, it can be present at a concentration of about 0.05 wt. % to about 5 wt. %, such as between about 0.1 wt. % and about 1 wt %, see U.S. Published Patent Application No. 2012/0219528, which is incorporated herein by reference. In one example, an aqueous-stabilizing formulation comprises a carbohydrate, such as sorbitol, at a concentration of between 0.1 wt. % to about 10 wt. %, such as between about 1 wt. % to about 5 wt. %. Nucleic acid molecules are generally present in the composition in an amount sufficient to provide a therapeutic effect when given in one or more doses, as defined above.

The mRNA can be included in an inert matrix for injection into the liver. As one example of an inert matrix, liposomes may be prepared from dipalmitoyl phosphatidylcholine (DPPC), such as egg phosphatidylcholine (PC). Liposomes, including cationic and anionic liposomes, can be made using standard procedures. In a formulation for intrahepatic injection, the liposome capsule degrades due to cellular digestion. Without being bound by theory, these formulations provide the advantages of a slow-release drug delivery system, exposing a subject to a substantially constant concentration of nucleic acid molecule over time. In one example, the nucleic acid molecule can be dissolved in an organic solvent, such as DMSO or alcohol, as previously described, and contain a polyanhydride, poly(glycolic) acid, poly(lactic) acid, or polycaprolactone polymer.

The mRNA may be formulated to permit release over a specific period of time. A release system can include a matrix of a biodegradable material or a material which releases the incorporated nucleic acid molecule by diffusion. The nucleic acid molecule can be homogeneously or heterogeneously distributed within the release system. A variety of release systems may be useful; however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that active ingredients having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other routine modifications), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other routine modifications), copolymers, and mixtures thereof.

Poly(lactide-co-glycolide) microspheres can also be used for intrahepatic injection. Typically the microspheres are composed of a polymer of lactic acid and glycolic acid, which are structured to form hollow spheres. The spheres can be approximately 15-30 microns in diameter and can be loaded with the biologicla molecules described herein.

The implants can be inserted into the liver by a variety of methods, which can influence the release kinetics. The location of the implanted device may influence the concentration gradients of the nucleic acid molecule surrounding the device and, thus, influence the release rates. Generally, when implants are used, the nucleic acid molecule is homogeneously distributed through the polymeric matrix, such that it is distributed evenly enough that no detrimental fluctuations in rate of release occur due to uneven distribution in the polymer matrix. The selection of the polymeric composition to be employed varies with the desired release kinetics, the location of the implant, patient tolerance, and the nature of the implant procedure. The polymer can be included as at least about 10 weight percent of the implant. In one example, the polymer is included as at least about 20 weight percent of the implant. In another aspect, the implant comprises more than one polymer. These factors are described in detail in U.S. Pat. No. 6,699,493. Characteristics of the polymers can include biodegradability at the site of implantation, compatibility with the agent of interest, ease of encapsulation, and water insolubility, among others. Generally, the polymeric matrix is not fully degraded until the drug load has been released. The chemical composition of suitable polymers is known in the art (for example, see U.S. Pat. No. 6,699,493). The nucleic acid molecule can be formulated in an implantable form with other carriers and solvents. For example, buffering agents and preservatives can be employed. The implant sizes and shape can also be varied for use in particular regions of the liver (see U.S. Pat. No. 5,869,079). In some aspects, a nanoparticle or dendrimer is used.

In several aspects, the mRNA is formulated in a lipid nanoparticle for administration to the recipient or donor liver (such as to a living donor prior to transplantation or to the donor liver in an ex vivo perfusion system); for example, comprising a PEG-modified lipid, a non-cationic lipid, a sterol, an ionizable lipid, or any combination thereof. In some aspects, the lipid nanoparticle is composed of 50 mol % ionizable lipid ((2 hydroxyethyl)(6 oxo 6-(undecycloxy)hexyl)amino)octanoate, 10 mol % 1,2 distearoyl sn glycerol-3 phosphocholine (DSPC), 38.5 mol % cholesterol, and 1.5 mol % 1-monomethoxypolyethyleneglycol-2,3,dimyristylglycerol with polyethylene glycol of average molecular weight 2000 (PEG2000 DMG). The mRNA/lipid nanoparticle composition may be provided in any suitable carrier, such as a sterile liquid for injection at a concentration of 0.5 mg/mL in 20 mM trometamol (Tris) buffer containing 87 mg/mL sucrose and 10.7 mM sodium acetate, at pH 7.5 and with appropriate diluent.

Local modes of administration include intrahepatic routes. In an aspect, significantly smaller amounts (compared with systemic approaches) may exert an effect when administered locally (for example, intrahepatically) compared to when administered systemically (for example, intravenously). Local modes of administration can reduce or eliminate the incidence of potential side effects. Methods for administration of nucleic acid molecules to the liver are known in the medical arts and can be used in the methods described herein. However, any route of admnistration is of use in the disclosed methods, including intrperiotoneal, instravenous, intrahepatic, or intramuscular.

Administration may be provided as a single administration, a periodic bolus (for example, intrahepatically) or as continuous infusion from an internal reservoir (for example, from an implant disposed at an intrahepatic location or from an external reservoir (for example, from an intravenous bag). Intrahepatic injection of the recombination mRNA disclosed herein can be performed once, or can be performed repeatedly, such as 1, 2, 3, 4, 5, 6, 7, 8, 9, 10, 20, 30, 40, 50, 60, 70, 80, 90, or 100 or more times. Administration can be performed biweekly, weekly, every other week, monthly, or every 2, 3, 4, 5, or 6 months.

Individual doses are typically not less than an amount required to produce a measurable effect on the subject and may be determined based on the pharmacokinetics and pharmacology of the subject composition or its by-products, and thus based on the disposition of the composition within the subject. This includes consideration of the route of administration as well as dosage amount, which can be adjusted for intraveinous or intrahepatic applications. Effective amounts of dose and/or dose regimen can readily be determined empirically from preclinical assays, from safety and escalation and dose range trials, individual clinician-patient relationships, as well as in vitro and in vivo assays.

In another aspect, the dosage is a daily dose. In another aspect, the dosage is a weekly dose. In another aspect, the dosage is a monthly dose. In another aspect, the dosage is an annual dose. In another aspect, the dose is one is a series of a defined number of doses. In another aspect, the dose is a one-time dose. As described below, in another aspect, an advantage of RNA, oligoribonucleotide, or polyribonucleotide molecules of the present disclosure is their greater potency, enabling the use of smaller doses.

Various aspects of dosage ranges of mRNA can be used in methods of the present disclosure. In one aspect, the dosage is in the range of about 0.1 to about 0.9 μg/day. In some aspects, the dosage range can be about 0.1 to about 0.8 μg/day, about 0.1 to about 0.7 μg/day, about 0.1 to about 0.6 μg/day, about 0.1 to about 0.5 μg/day, about 0.1 to about 0.4 μg/day, about 0.1 to about 0.3 μg/day, or about 0.1 to about 0.2 μg/day. In more aspects, the dosage range can be about 0.2 to about 0.9 μg/day, about 0.3 to about 0.9 μg/day, about 0.4 to about 0.9 μg/day, about 0.5 to about 0.9 μg/day, about 0.6 to about 0.9 μg/day, about 0.7 to about 0.9 μg/day, or about 0.8 to about 0.9 μg/day. The dose can be about 0.1 μg/day, about 0.2 μg/day, about 0.3 μg/day, about 0.4 μg/day, about 0.5 μg/day, about 0.6 μg/day, about 0.7 μg/day, about 0.8 μg/day or about 0.9 μg/day.

In one aspect, the dosage is in the range of 1-10 μg/day. In another aspect, the dosage is 2-10 μg/day. In another aspect, the dosage is 3-10 μg/day. In another aspect, the dosage is 5-10 μg/day. In another aspect, the dosage is 2-20 μg/day. In another aspect, the dosage is 3-20 μg/day. In another aspect, the dosage is 5-20 μg/day. In another aspect, the dosage is 10-20 μg/day. In another aspect, the dosage is 3-40 μg/day. In another aspect, the dosage is 5-40 μg/day. In another aspect, the dosage is 10-40 μg/day. In another aspect, the dosage is 20-40 μg/day. In another aspect, the dosage is 5-50 μg/day. In another aspect, the dosage is 10-50 μg/day. In another aspect, the dosage is 20-50 μg/day. In one aspect, the dosage is 1-100 μg/day. In another aspect, the dosage is 2-100 μg/day. In another aspect, the dosage is 3-100 μg/day. In another aspect, the dosage is 5-100 μg/day. In another aspect the dosage is 10-100 μg/day. In another aspect the dosage is 20-100 μg/day. In another aspect the dosage is 40-100 μg/day. In another aspect the dosage is 60-100 μg/day.

In another aspect, the dosage is 0.1 μg/day. In another aspect, the dosage is 0.2 μg/day. In another aspect, the dosage is 0.3 μg/day. In another aspect, the dosage is 0.5 μg/day. In another aspect, the dosage is 1 μg/day. In another aspect, the dosage is 2 mg/day. In another aspect, the dosage is 3 μg/day. In another aspect, the dosage is 5 μg/day. In another aspect, the dosage is 10 μg/day. In another aspect, the dosage is 15 μg/day. In another aspect, the dosage is 20 μg/day. In another aspect, the dosage is 30 μg/day. In another aspect, the dosage is 40 μg/day. In another aspect, the dosage is 60 μg/day. In another aspect, the dosage is 80 μg/day. In another aspect, the dosage is 100 μg/day.

In another aspect, the dosage is 10 μg/dose. In another aspect, the dosage is 20 μg/dose. In another aspect, the dosage is 30 μg/dose. In another aspect, the dosage is 40 μg/dose. In another aspect, the dosage is 60 μg/dose. In another aspect, the dosage is 80 μg/dose. In another aspect, the dosage is 100 μg/dose. In another aspect, the dosage is 150 μg/dose. In another aspect, the dosage is 200 μg/dose. In another aspect, the dosage is 300 μg/dose. In another aspect, the dosage is 400 μg/dose. In another aspect, the dosage is 600 μg/dose. In another aspect, the dosage is 800 μg/dose. In another aspect, the dosage is 1000 μg/dose. In another aspect, the dosage is 1.5 mg/dose. In another aspect, the dosage is 2 mg/dose. In another aspect, the dosage is 3 mg/dose. In another aspect, the dosage is 5 mg/dose. In another aspect, the dosage is 10 mg/dose. In another aspect, the dosage is 15 mg/dose. In another aspect, the dosage is 20 mg/dose. In another aspect, the dosage is 30 mg/dose. In another aspect, the dosage is 50 mg/dose. In another aspect, the dosage is 80 mg/dose. In another aspect, the dosage is 100 mg/dose.

In another aspect, the dosage is 10-20 μg/dose. In another aspect, the dosage is 20-30 μg/dose. In another aspect, the dosage is 20-40 μg/dose. In another aspect, the dosage is 30-60 μg/dose. In another aspect, the dosage is 40-80 μg/dose. In another aspect, the dosage is 50-100 μg/dose. In another aspect, the dosage is 50-150 μg/dose. In another aspect, the dosage is 100-200 μg/dose. In another aspect, the dosage is 200-300 μg/dose. In another aspect, the dosage is 300-400 μg/dose. In another aspect, the dosage is 400-600 μg/dose. In another aspect, the dosage is 500-800 μg/dose. In another aspect, the dosage is 800-1000 μg/dose. In another aspect, the dosage is 1000-1500 μg/dose. In another aspect, the dosage is 1500-2000 μg/dose. In another aspect, the dosage is 2-3 mg/dose. In another aspect, the dosage is 2-5 mg/dose. In another aspect, the dosage is 2-10 mg/dose. In another aspect, the dosage is 2-20 mg/dose. In another aspect, the dosage is 2-30 mg/dose. In another aspect, the dosage is 2-50 mg/dose. In another aspect, the dosage is 2-80 mg/dose. In another aspect, the dosage is 2-100 mg/dose. In another aspect, the dosage is 3-10 mg/dose. In another aspect, the dosage is 3-20 mg/dose. In another aspect, the dosage is 3-30 mg/dose.

In another aspect, the dosage is 3-50 mg/dose. In another aspect, the dosage is 3-80 mg/dose. In another aspect, the dosage is 3-100 mg/dose. In another aspect, the dosage is 5-10 mg/dose. In another aspect, the dosage is 5-20 mg/dose. In another aspect, the dosage is 5-30 mg/dose. In another aspect, the dosage is 5-50 mg/dose. In another aspect, the dosage is 5-80 mg/dose. In another aspect, the dosage is 5-100 mg/dose. In another aspect, the dosage is 10-20 mg/dose. In another aspect, the dosage is 10-30 mg/dose. In another aspect, the dosage is 10-50 mg/dose. In another aspect, the dosage is 10-80 mg/dose. In another aspect, the dosage is 10-100 mg/dose.

The recombinant mRNA can be used alone. However, in another aspect, at least one additional agent can be included along with the nucleic acid molecule in the composition. The composition, such as an implant, is then introduced into the liver.

Nucleic acid molecules can be delivered, by microinjection, electroporation, lipid-mediated transfection, peptide-mediated delivery, nanoparticle mediated delivery (such as lipid or polymeric nanoparticle mediate delivery), in association with a degradable polymer, as an mRNA-Lipoplex, as mRNA cargo of PEG-10, or other methods known in the art. In some aspects, the mRNA, such as a recombinant mRNA encoding HNF4α2, is administered using lipid nanoparticles (LNP), using polymeric nanoparticles, as a conjugate to GalNAc, as an mRNA modified by base linker sugars, using a degradable polymer, as an mRNA-Lipoplex, or as mRNA cargo of PEG-10. In a specific, non-limiting example, the mRNA is administered to the using lipid nanoparticles. An appropriate dose depends on the subject being treated (e.g., human or nonhuman primate or other mammal), age and general condition of the subject to be treated, the severity of the condition being treated, the mode of administration, among other factors. An appropriate effective amount can be readily determined by one of skill in the art. Thus, a “therapeutically effective amount” will fall in a relatively broad range that can be determined through clinical trials.

Methods are disclosed for treating a subject with end stage liver disease. In some aspects, a subject is selected that has end stage liver failure. The subject can have liver failure as a result of cirrhosis. The subject can have cirrhosis resulting from alcohol-related liver disease. The subject can have non-alcoholic steatohepatitis. The subject can have chronic hepatitis. The subject can have nonalcoholic fatty liver disease (NALFD). The subject can have a disease that destroys bile ducts (such as biliary cirrhosis). The subject can have a genetic abnormality, such as cystic fibrosis, alpha-1 antitrypsin deficiency, hemochromatosis, Wilson disease, galactosemia, or a glycogen storage disease. The subject can have liver failure as a result of an exposure, such as to a drug or toxic chemical. The subject can have a parasitic infection that results in liver failure. Any of these subjects can be selected for treatment.

In some aspects, these methods include administering to the subject a therapeutically effective amount of a recombinant mRNA encoding HNF4α2, thereby treating the end-stage liver disease in the subject. In some aspects, the method increases serum albumin, decreases serum ammonia, improve coagulation activity, decreases ascites production, improves neuropsychological status, decreases bilirubin, improves apolipoprotein and/or improves portal vein or systemic blood flow clearance of cholate in the subject.

In further aspects, these methods include administering to the subject a therapeutically effective amount of a recombinant mRNA encoding HNF4α1, thereby treating liver disease in the subject. In more aspects, these methods include administering to the subject a therapeutically effective amount of a recombinant mRNA encoding HNF4α1, thereby treating end stage liver disease in the subject. In some aspects, the method increases serum albumin, decreases serum ammonia, improve coagulation activity, decreases ascites production, improves neuropsychological status, decreases bilirubin, improves apolipoprotein and/or improves portal vein or systemic blood flow clearance of cholate in the subject.

The methods can improve symptoms, such as weakness, fatigue, loss of appetite, nausea, vomiting, weight loss, abdominal pain, bloating, or itching. Treatment can also result in improvements in creatine level, bilirubin level, or an international normalized ratio (INR)-test for the clotting tendency of blood, increases serum albumin, decreases serum ammonia, improve ascites, neuropsychological status, improve levels of apolipoproteins and/or portal vein blood flow clearance of cholate.

Generally, these parameters are improved compared to the parameter as measured in a subject prior to treatment. However, the control also can be a standard value, or the value obtained for a population of subjects with liver failure.

The subject can be selected using Child-Pugh scoring. The Child-Pugh score utilizes five clinical measures, wherein levels of total bilirubin, serum albumin, prothrombin time prolongation, ascites, and hepatic encephalopathy, are scored using a point system of 1 point, 2 point, and 3 point values for varying levels of each clinical measure, with 3 point values being assigned to the most severe levels of each measure. The total points for all five measures are added to arrive at a Child-Pugh score and classification. Scores of 5-6 designate Child-Pugh Class A, scores of 7-9 designate Child-Pugh Class B, and scores of 10-15 designate Child-Pugh Class C. In general, Child-Pugh Class A indicates the least severe liver disease and Child-Pugh Class C indicates the most severe liver disease. Accordingly, in some aspects, the method disclosed herein can be used to treat a subject having a Child-Pugh Class A, Child-Pugh Class B or Child-Pugh Class C liver disease. In some aspects, the method disclosed here in can be used to treat a subject having a Child-Pugh Class C liver disease. In various aspects, the method improves the Child-Pugh score of the subject. Thus, in some aspects, the method can include determining the Child-Pugh score of the subject.

The disclosed methods can be performed to the subject any time throughout their evaluation of liver function using the Child-Pugh classification and/or if the subject undergo liver resection. In one aspect, the disclosed methods can be employed 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6,5, 4, or 3 days; 60, 48, 36, 30, 24, 18, 15, 12, 10, 9, 8, 7, 6, 5, 4, 3, or 2 hours prior to the operation of liver resection; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after liver resection.

A method for intravascular regional hydrodynamic delivery of nucleic acid molecules has been developed, and is of use with the methods disclosed herein. The method entails the use of an occlusion balloon catheter into the inferior vena cava and retro dynamically injecting the nucleic acid molecule in saline solution towards the liver and through the hepatic vein. This retrodynamic hepatic vein gene delivery method has been performed in pigs, and led to liver transgene expression (Crespo A, Gene Ther. 2005; 12:927-935; Eastman S J, Hum Gene Ther. 2002; 13:2065-2077; Brunetti-Pierri N, Mol Ther. 2007; 15:732-740; Dariel A, J Pediatr Surg. 2009; 44:517-522).

Retrograde administration of nucleic acid molecules into the common bile duct has been shown to induce efficient transgene expression in the liver without causing severe adverse effects, thus supporting the feasibility of gene transfer into the liver in clinical settings by means of endoscopic retrograde cholangiography. (Kuriyama S, Int J Mol Med. 2005; 16:503-508; Kuriyama S, Oncol Rep. 2005; 13:825-830; Peeters M J, Hum Gene Ther. 1996; 7:1693-1699). Repeat administration into the common bile duct was successful in re-expressing the transgene in the liver. (Tominaga K, Gut. 2004; 53:1167-1173; Tsujinoue H, Int J Oncol. 2001; 18:575-580). Thus, this route of administration is of use with the methods disclosed herein.

Another method for delivery of gene products into liver cells is also described in U.S. Patent Application Publication No. 20100010068. This method involves limiting blood flow to the liver during infusion of the vector into the liver. These steps are also of use in the methods disclosed herein.

The disclosed methods can include measuring liver function and/or survival using a quantitative and/or qualitative test. In some aspects, the degree of liver impairment is assessed using tests which evaluate structure (e.g., biopsy), cellular permeability (e.g., transaminases) and synthetic ability (e.g., albumin, bilirubin and prothrombin time) (see Jalan and Hayes (1995) Aliment. Pharmacol. Ther. 9:263-270). A combination of various markers for liver injury can be measured to provide an analysis function. Commonly used tests for liver clearance capability are: indocyanine green (ICG), galactose elimination capacity (GEC), mono-ethyl-glycine-xylidide (MEG-X), antipryine clearance, aminopyrine breath test (ABT) and caffeine clearance. For assessment of graft function following transplantation, low ICG clearance and low MEG-X formation are predictive of a poor outcome. The method can also include measuring the lipid profile of a subject. The method can include measuring liver size, such as using ultrasound.

### EXAMPLES

HNF4α has 12 isoforms generated through two promoters (P1 and P2) and alternative splicing. For instance, P2-HNF4α isoforms produced from the distal P2 promoter are expressed in fetal liver and in different liver disease states (e.g. hepatomas, hepatocellular carcinoma) are involved in early liver development. P2-HNF4α isoforms are not normally expressed in adult liver, but their expression has been implicated in the pathogenesis of hepatocellular carcinoma, and alcoholic hepatitis. P1-HNF4α isoforms produced from the proximal P1 promoter are highly expressed in adult liver and are involved in hepatocyte maturation and function. HNF4α2 is the predominant P1-HNF4α isoform in human adult liver. It is disclosed herein that a recombinant mRNA encoding HNF4α2 can be used to treat liver failure.

### Example 1

### Materials and Methods

Production of the mRNA encoding the human hepatocyte nuclear factor 4α variant 2 (mRNA-HNF4α2): Codon optimized human hepatocyte nuclear factor 4α variant 2 (human HNF4α2) was cloned downstream of a Kozak sequence into an mRNA production plasmid (optimized 3′ and 5′ UTR and containing a 101 polyA tail), in vitro transcribed in the presence of the presence of N¹-methylpseudouridine modified nucleoside (N¹mψ), co-transcriptionally capped using the CLEANCAP™ technology (TriLink) and cellulose purified to remove dsRNA. Purified mRNA was ethanol precipitated, washed, resuspended in nuclease-free water, and subjected to quality control (electrophoresis, dot blot, and transfection into human DCs). mRNA was stored at −20° C. until use.

Production of LNP-mRNA: mRNA-HNF4α2 or mRNA-eGFP loaded LNPs were formulated using a total lipid concentration of 40 mM as previously described (Maier et al., 2013, PMID 23799535) the ethanolic lipid mixture comprising ionizable cationic lipid, phosphatidylcholine, cholesterol, and polyethylene glycol-lipid was rapidly mixed with an aqueous solution containing cellulose-purified NI-mT in vitro transcribed HNF4α2 variant 2 mRNA (PMID 23799535). The LNP formulation used in this study is proprietary to Acuitas Therapeutics; the proprietary lipid and LNP composition are described in US patent U.S. Pat. No. 10,221,127. mRNA-loaded particles were characterized and subsequently stored at −80° C. at an RNA concentration of 1 mg/ml. The mean hydrodynamic diameter of mRNA-LNPs was ~80 nm with a polydispersity index of 0.02-0.06 and an encapsulation efficiency of ~95%. Two or three batches from each mRNA-LNP formulations were used in these studies.

Human hepatocyte isolation: Primary hepatocytes were isolated from explanted liver specimens obtained from patients receiving orthotopic liver transplantation for decompensated liver cirrhosis with end-stage liver disease. Liver tissue specimens were protected from ischemic injury by flushing with ice-cold University of Wisconsin (UW) solution immediately after vascular clamping and re-section in the operating room, keeping the specimens on ice, and transporting the specimens immediately to the laboratory. Hepatocytes were isolated from encapsulated human liver segments (preferably the left lateral segment) by a modified three-step perfusion technique. Briefly, the livers were flushed under a sterile biosafety hood through the portal vein and/or hepatic vessels (re-circulation technique) with 1 L of calcium-free HBSS (Sigma, Saint Louis, MO) supplemented with 0.5 mM EGTA (Thermo Fisher Scientific, Waltham, MA) pre-warmed to 37° C. and then with collagenase/protease solution (VitaCyte, Indianapolis, IN) pre-warmed to 37° C. until the tissue was fully digested. The digestion time for each preparation was in a range 45-60 min. The digested liver was removed and immediately cooled with ice-cold Leibovitz's L-15 Medium (Invitrogen, Carlsbad, CA) supplemented with 10% FBS (Sigma, Saint Louis, MO). The final cell suspension was centrifuged twice at 65×g for 3 min at 4° C. and the medium was aspirated. The yield and viability of freshly isolated hepatocytes were estimated by trypan blue staining.

Human hepatocyte culture: Final cell suspensions were centrifuged one more time at 65×g for 3 min at 4° C. and the medium was aspirated. The cells were resuspended in HMM medium (Basal Medium plus SingleQuots) (Lonza, Walkersville, MD) supplemented with 5% FBS. Cells were then dispensed into 12-well plates pre-coated with 50 ug/mL collagen (Corning, Oneonta, NY) at a density of 5×10⁵ cells/well and incubated at 37° C. and 5% CO₂ for 4 hours.

Treatment with mRNA-HNF4α2: For lipofectamine-mediated transfection experiments, hepatocytes were allowed to attach to collagen coated plates for 4 hours. Afterwards, the cells were washed twice with PBS (Invitrogen, Carlsbad, CA) and the culture medium in each well was then replaced with fresh 500 μL of HMM. A solution of 100 uL Opti-MEM (Invitrogen, Carlsbad, CA), 3 uL Lipofectamine MessengerMAX Transfection Reagent (Invitrogen, Carlsbad, CA), and 0, 0.1, 0.25, or 0.5 ug of mRNA-HNF4α2 (sequenced indicated above) was prepared according to Invitrogen's instructions and was added into each well.

For LNP-mediated delivery, 0.25, 1 or 2 μg of LNPs loaded with either mRNA-HNF4α2 or mRNA-eGFP were mixed with cells during seeding and incubated for at least 12 hours at 37° C. and 5% CO₂. Wells were then washed twice with PBS and the medium was replaced with fresh 1 mL HMM. Samples for RNA isolation, protein lysate isolation, and 4% PFA fixation were collected at 12, 24, 48 or 72 hours post-treatment. For transfection efficiency, brightfield and fluorescent images were taken from cells treated with eGFP-mRNA-LNP at 12, 24, 48 and 72 hours post-treatment using the EVOS M5000 Imaging System (Thermo Fisher Scientific, Waltham, MA).

Quantitative real time PCR: Details regarding the gene expression assays used are listed in the Table 1. Total RNA was isolated from hepatocytes using RNeasy Mini kits (QIAGEN, Hilden, Germany) and reverse transcribed using SuperScript III (Invitrogen, Carlsbad, CA) following the manufacturers' instructions. qPCR reactions were prepared by mixing 1λ TaqMan Fast Advanced Master Mix (Life Technologies, Waltham, MA), 1λ TaqMan gene expression assays (Applied Biosystems, Foster City, CA), 100 ng of cDNA, and nuclease-free water (Invitrogen, Carlsbad, CA) to a final volume of 20 gL. qPCR was then performed using a StepOnePlus system (Applied Biosystems, Foster City, CA). Relative gene expression was calculated following the ααCT method using β-actin (ACTB) as a reference gene.

Western blot: Details regarding the antibodies and their corresponding dilutions are listed in Table 2. All samples were incubated with RIPA lysis buffer (Sigma-Aldrich, Saint Louis, MO) containing 1× HALT™ Protease and Phosphatase Inhibitor Cocktail (Thermo Fisher Scientific, Waltham, MA) and incubated for 30 min at 4° C. Samples were centrifuged at 13,000×g for 10 min at 4° C. The supernatant from each sample was then transferred to a new microfuge tube and was used as the whole cell lysate. Protein concentrations were determined by comparison with a known concentration of bovine serum albumin using a Pierce BCA Protein Assay Kit (Thermo Fisher Scientific, Waltham, MA). About 30 μg of lysate were loaded per lane into 10% Mini-PROTEAN TGX gel (BioRad, Hercules, CA). After SDS-PAGE, proteins were transferred onto PVDF membrane (Thermo Fisher Scientific, Waltham, MA). Membranes were incubated with primary antibody solution overnight and then washed. Membranes were incubated for 1 hour in secondary antibody solution and then washed. Target antigens were finally detected using SUPERSIGNA1™ West Pico PLUS chemiluminescent substrate (Thermo Fisher Scientific, Waltham, MA). Images were scanned and analyzed using ImageJ software.

Immunohistochemistry and immunofluorescence staining: Details regarding the antibodies and their corresponding dilutions are listed in Table 3. For immunohistochemistry, paraffin-embedded liver tissue was deparaffinized with xylenes and dehydrated with ethanol. Antigen unmasking was performed by boiling in citrate buffer, pH 6.0. The slides were then incubated in 3% hydrogen peroxide, blocked with normal animal serum, and subsequently left incubating overnight at 4° C. with primary antibodies (anti-LDLR or anti-LRP1). Tissue sections were then incubated with the secondary biotinylated antibody corresponding to the animal species of the primary antibody (Vector Laboratories, Burlingame, CA) and exposed to 3,3′-diaminobenzidine (Vector Laboratories, Burlingame, CA) to visualize the peroxidase activity. Counterstaining was performed with Richard-Allan Scientific Signature Series Hematoxylin (Thermo Scientific, Waltham, MA). Control tissues were used for validation of antibodies used in this study. For immunofluorescence staining, primary human hepatocytes were washed with warm PBS, fixed with 4% PFA for 15 min, and washed 3× with PBS. Samples were washed 3× with wash buffer (PBS, 0.1% BSA, and 0.1% TWEEN 20) for 5 min and then blocked and permeabilized in blocking buffer (PBS, 10% normal donkey serum, 1% BSA, 0.1% TWEEN 20, and 0.1% Triton X-100) for 1 hour at room temperature. Samples were then incubated with primary antibody (anti-HNF4α2) in blocking buffer for 24 hours at 4° C. Samples were washed 3× with wash buffer for 5 min and incubated with secondary antibody in blocking buffer for 1 hour in the dark at room temperature. Samples were washed 3× with wash buffer for 5 mi 3× with PBS, and then counterstained with 1 μg/mL of Hoechst 33342 (Sigma, Saint Louis, MO) for 1 min at room temperature in the dark. Samples were washed 3× with PBS and stored in 2 mL PBS in the dark at 4° C.

Imaging and image analysis: Samples were imaged using an Eclipse Ti inverted microscope (Nikon, Melville, NY) and the NIS-Elements software platform (Nikon, Melville, NY). For each sample, images were taken from three random fields at a magnification of 200λ. Analysis of images was done using ImageJ program (NIH) using a standard automated cell counting of single-color images (Schneider et al., Nat Methods 9, 671-675 (2012)).

Statistical analysis: Data are expressed as mean±standard deviation (SD). Statistical significance between multiple groups were determined by Welch's ANOVA followed by Dunn's Multiple Comparison post hoc test using Prism 6.0 (GraphPad Software Inc., San Diego, CA). Values of p<0.05 were considered statistically significant.

TABLE 1
TaqMan gene expression assay IDs used for qPCR

| Target | Gene Expression Assay | | |
|---|---|---|---|
| Gene | ID/Catalogue No. | Dye | Quencher |
| LDLR | Hs05050300_s1 | FAM | MGB |
| LRP1 | Hs00233856_m1 | FAM | MGB |
| ACTB | Hs01060665_g1 | FAM | MGB |

TABLE 2
Antibodies and dilutions used for western blot,
immunohistochemistry, and immunofluorescence staining
Western Blot

| | | | Catalogue | |
|---|---|---|---|---|
| Antibody | Host | Company | No. | Dilution |
| anti-HNF4α | mouse | Abcam | Ab41898 | 1:1000 |
| anti-albumin | goat | Bethyl | A80-229A | 1:1000 |
| anti-UGT1A1 | rabbit | Abcam | Ab237810 | 1:2000 |
| anti-CF VII | rabbit | Abcam | Ab151543 | 1:1000 |
| anti-OTC | mouse | Abcam | Ab180401 | 1:1000 |
| anti-LDLR | rabbit | Invitrogen | PA522976 | 1:1000 |
| anti-LRP1 | rabbit | Abcam | Ab92544 | 1:1000 |
| anti-ACTB | rabbit | Cell Signaling Tech | 4970 | 1:1000 |
| anti-GAPDH | mouse | Proteintech | 60004-1-Ig | 1:10000 |
| anti-mouse IgG- | goat | EMD Millipore | 12-349 | 1:10000 |
| HRP | | | | |
| anti-rabbit IgG- | goat | Thermo Fisher | 31460 | 1:20000 |
| HRP | | Scientific | | |
| anti-goat IgG- | donkey | Thermo Fisher | A-1105 | 1:10000 |
| HRP | | Scientific | | |

TABLE 3
Immunohistochemistry and Immunofluorescence staining

| | | | Catalogue | |
|---|---|---|---|---|
| Antibody | Host | Company | No. | Dilution |
| anti-HNF4α | mouse | Abcam | Ab41898 | 1:1000 |
| anti-LDLR | rabbit | Thermo Fisher | MA5-32075 | 1:50 |
| | | Scientific | | |
| anti-LRP1 | rabbit | Abcam | Ab92544 | 1:100 |
| anti-rabbit IgG-AF594 | donkey | Invitrogen | A-21207 | 1:250 |
| anti-mouse IgG-AF594 | donkey | Invitrogen | A-21203 | 1:250 |

### Example 2

### End-Stage Cirrhotic Hepatocytes Express the Lipid Nanoparticle Receptors, LDLR and LRP1

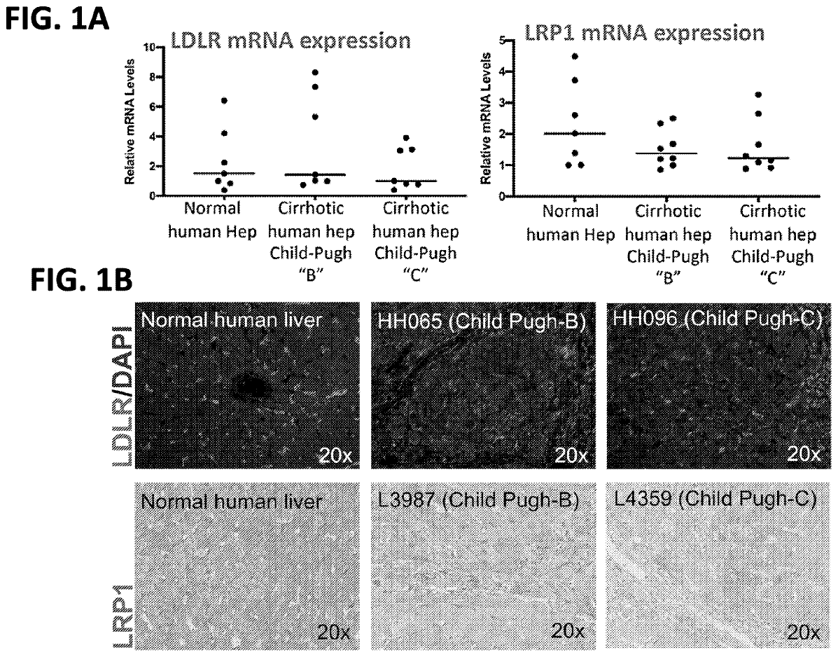

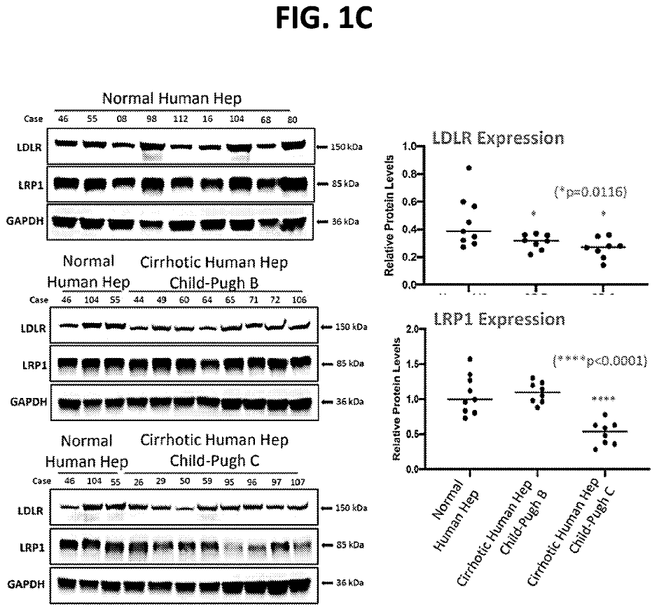

It was previously shown that in vitro transfection of end-stage cirrhotic hepatocytes with HNF4α2 mRNA corrects the disrupted transcriptional network and normalizes the expression of genes important for hepatocyte function (Tafaleng et al., Hepatol Commun 5, 1911-1926 (2021)). However, a safe, efficient, and stable delivery system that prevents mRNA degradation and allows cellular uptake of mRNA is necessary for clinical application of mRNA-based therapeutics. Lipid nanoparticles (LNPs) have recently emerged as a promising nonviral system for clinical in vivo delivery of nucleic acids (Kulkarni et al., Nucleic Acid Ther 28, 146-157 (2018); Thi et al., Vaccines (Basel) 9 (2021)). Because the LDL Receptor (LDLR) and the LDL Receptor Related Protein 1 (LRP1) are essential for hepatocyte-specific in vivo uptake of LNPs (Akinc, et al., Mol Ther 18, 1357-1364 (2010); Gilleron et al., Nat Biotechnol 31, 638-646 (2013)), the expression of these receptors was first assessed in isolated human hepatocytes from end-stage cirrhotic patients with varying degrees of disease severity (Child-Pugh B or C) compared to normal controls. Cirrhotic human livers were acquired from patients undergoing liver transplantation for end-stage liver disease suffering from non-alcoholic steatohepatitis (NASH) or alcoholic cirrhosis. Normal controls were obtained from liver resections. qPCR analysis for LDLR and LRP1 in normal isolated hepatocytes (n=7) and cirrhotic isolated hepatocytes classified as Child-Pugh B (n=7) and Child-Pugh C(=7) did not show any significant difference in LDLR or LRP1 mRNA levels between groups (FIG. 1A) (LDLR p=0.41, LRP1 p=0.27, Welch's ANOVA). Immunostaining for LDLR and LRP1 in normal and cirrhotic liver tissues revealed detectable expression in all liver specimens although there is a slight reduction in the expression of these receptors in cirrhotic liver tissues (FIG. 1B). Western blot analysis of the protein levels of LDLR and LRP1 in normal human isolated hepatocytes (n=9) and cirrhotic human hepatocytes classified as Child-Pugh B (n=8) and Child-Pugh C (n=8) confirmed the results of immunostaining (FIG. 1C). LDLR expression was decreased in both cirrhotic hepatocyte cohorts in comparison with the normal controls (p=0.0116, Welch's ANOVA). In contrast, LRP1 expression was only decreased in cirrhotic hepatocytes from the most advanced stage of the end-stage liver disease (Child-Pugh C) compared to normal controls (p<0.0001, Dunn's Multiple Comparison). These results confirm the expression of necessary receptors for LNP uptake in end-stage cirrhotic hepatocytes.

### Example 3

### LNP FM-1520A Shows a High Delivery Efficiency in End-Stage Cirrhotic Hepatocytes

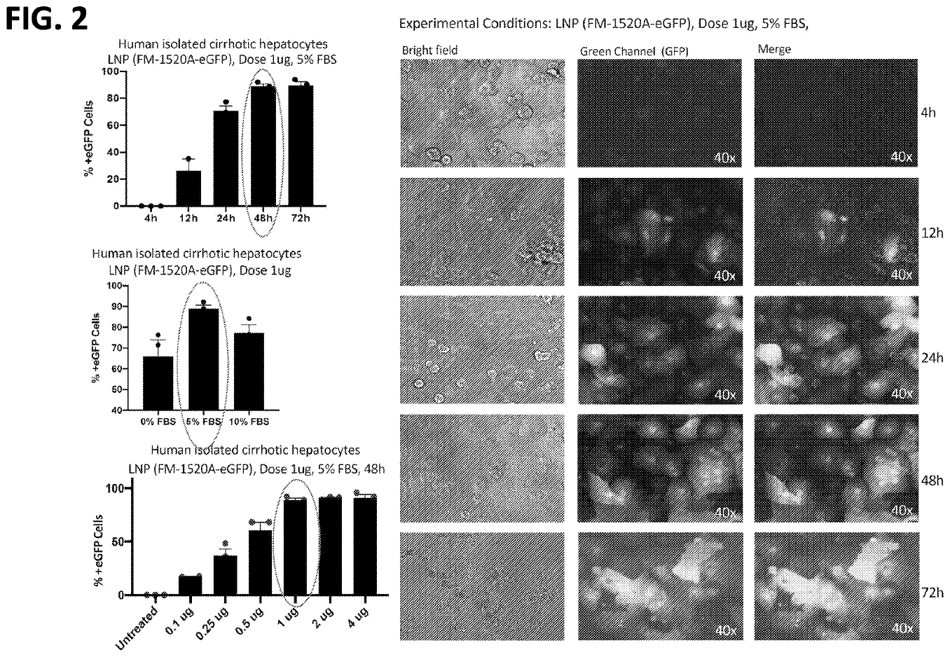

The optimal conditions were determined for LNP delivery in end-stage cirrhotic hepatocytes by evaluating various LNP doses, incubation times, and concentrations of FBS in the cell culture media. For this analysis, an LNP formulation (LNP FM-1520A) that carried the enhanced green fluorescent protein (eGFP) mRNA was used. Primary human hepatocytes were isolated from the cirrhotic liver of a 59 year-old (yo) male patient who needed liver transplantation for end-stage liver disease caused by non-alcoholic steatohepatitis (NASH). Different doses of LNP-mRNA-eGFP (0.1-4μg) and FBS concentration (0-10%) were evaluated from 4-72 hours after addition of LNPs. The efficiency of LNP delivery was assessed by obtaining the percentage of eGFP+ cells for each condition. An LNP dose of 1 μg led to greater than 90% eGFP+ hepatocytes with doses above 1 μg not showing any further increases in the percentage of eGPF+hepatocytes (FIG. 2). The presence of 5% FBS showed increased delivery efficiency (90%) compared to 0% and 10% FBS (65% and 80%, respectively) (FIG. 2). The highest percentage of eGFP+ hepatocytes were observed at 48 and 72 hours (greater than 90%) (FIG. 2). Immunofluorescence micrographs show representative images of eGFP expression 4-72 hours after delivery of 1 μg LNP-mRNA-eGFP in the presence of 5% FBS (FIG. 2). Treatment of end-stage cirrhotic hepatocytes with 1 μg of LNP-mRNA-eGFP in the presence of 5% FBS showed greater than 90% delivery efficiency at 48 and 72 hours post treatment.

### Example 4

### The mRNA-HNF4α2 Construct Exhibits High Bioactivity in End-Stage Cirrhotic Hepatocytes

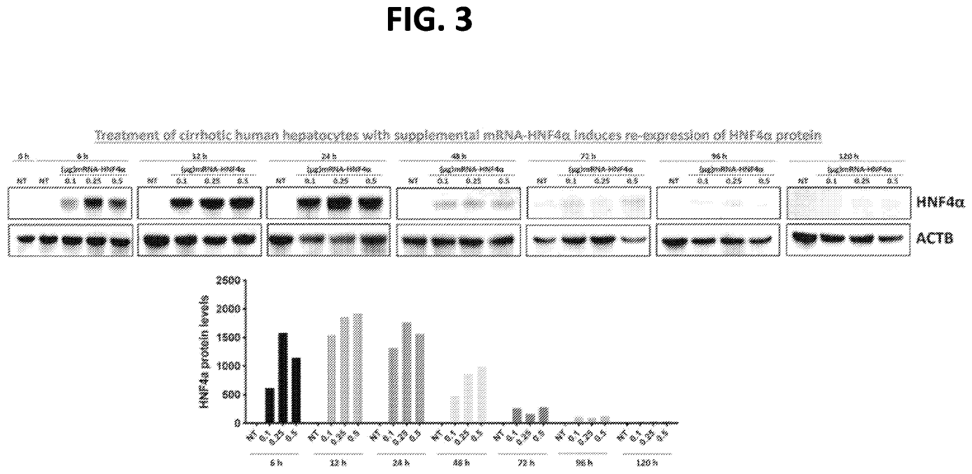

The ability of the mRNA-HNF4α2 construct to upregulate HNF4α2 and albumin, a downstream target of HNF4α2, was tested in end-stage cirrhotic hepatocytes. For this analysis, mRNA-HNF4α2 was delivered using lipofectamine to study the effectiveness of the construct independent of LNP delivery. Primary human hepatocytes were isolated from the cirrhotic liver of a 67 year-old female patient who required liver transplantation for end-stage liver disease due to non-alcoholic steatohepatitis (NASH). End-stage cirrhotic hepatocytes were transfected with various doses of mRNA-HNF4α2 construct (0.1-0.5 ug) and the expression of HNF4α2 and albumin protein were analyzed 6-120 hours post transfection (FIG. 3). Western blot analysis revealed that HNF4α2 protein was detectable from 6-96 hours after transfection with maximal expression at 12 hours after transfection (FIG. 3). Without transfection of mRNA-HNF4α2, the HNF4α2 protein levels were undetectably low before (0 hours) and during treatment (6 h-120 hours) compared to a normal hepatocyte control (FIG. 3). More importantly, the transfection of mRNA-HNF4α2 led to the upregulation of albumin protein levels in a dose dependent manner 24 hours after transfection (FIG. 3). These results provide evidence that the mRNA-HNF4α2 construct effectively increases HNF4α2 protein levels which subsequently leads to the upregulation of albumin in end-stage cirrhotic hepatocytes.

### Example 5

### Treatment of End-Stage Cirrhotic Hepatocytes with LNP-mRNA-HNF4α2 Results in Increased HNF4α2

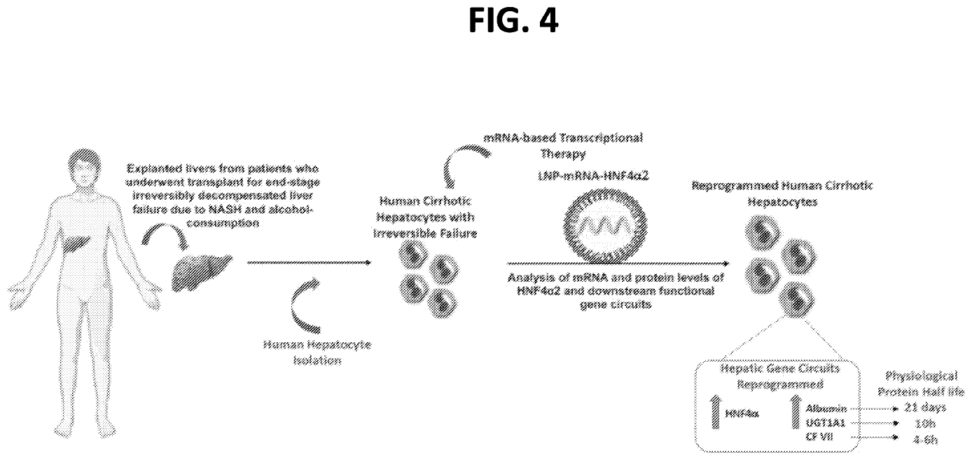

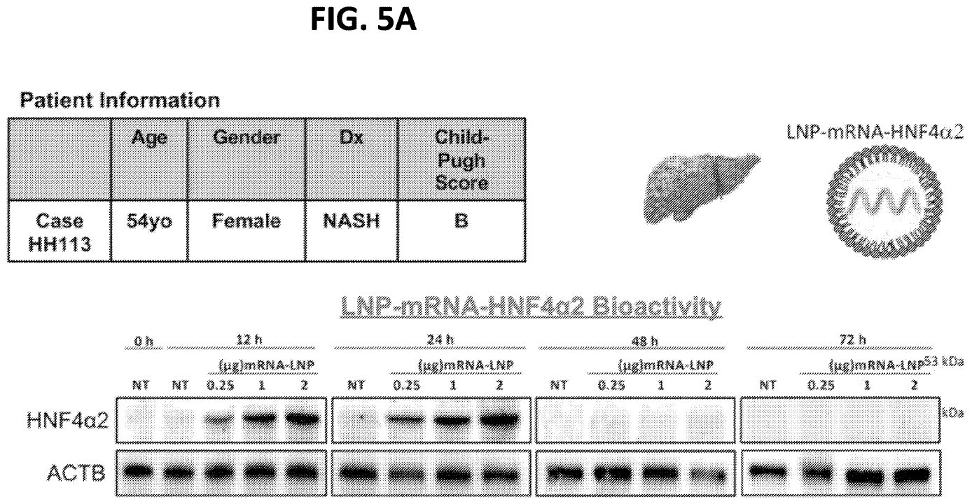

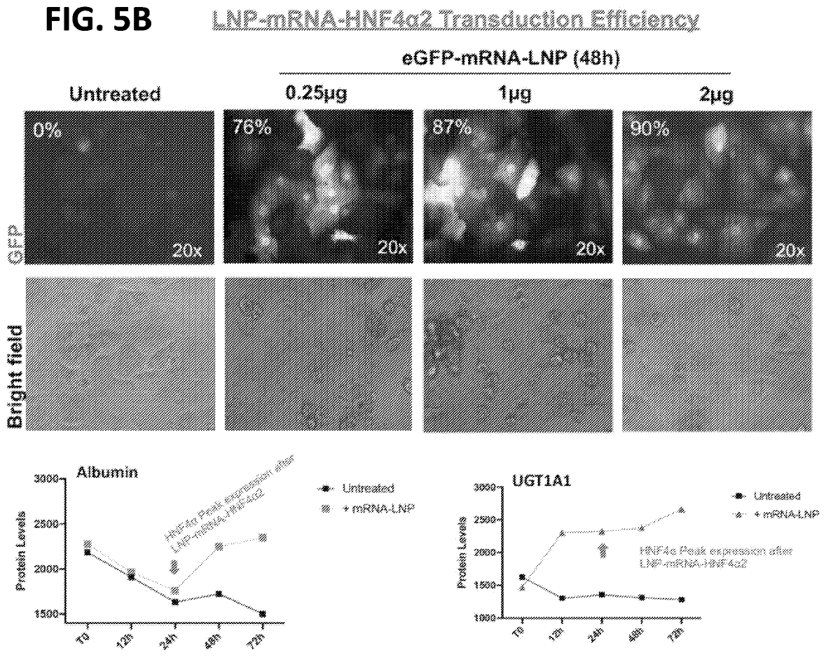

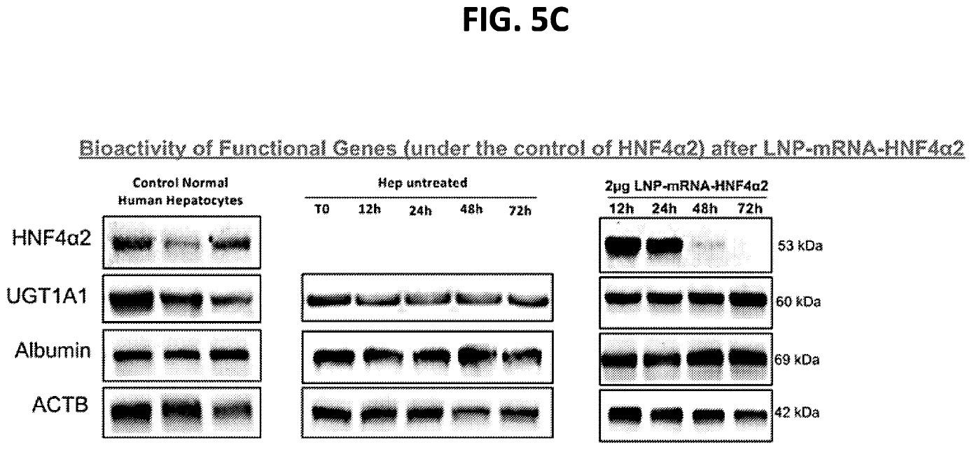

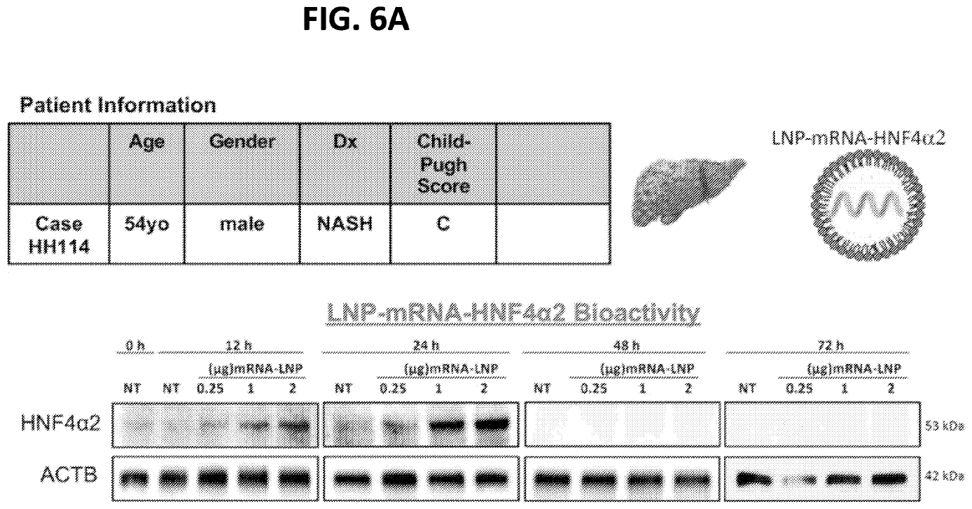

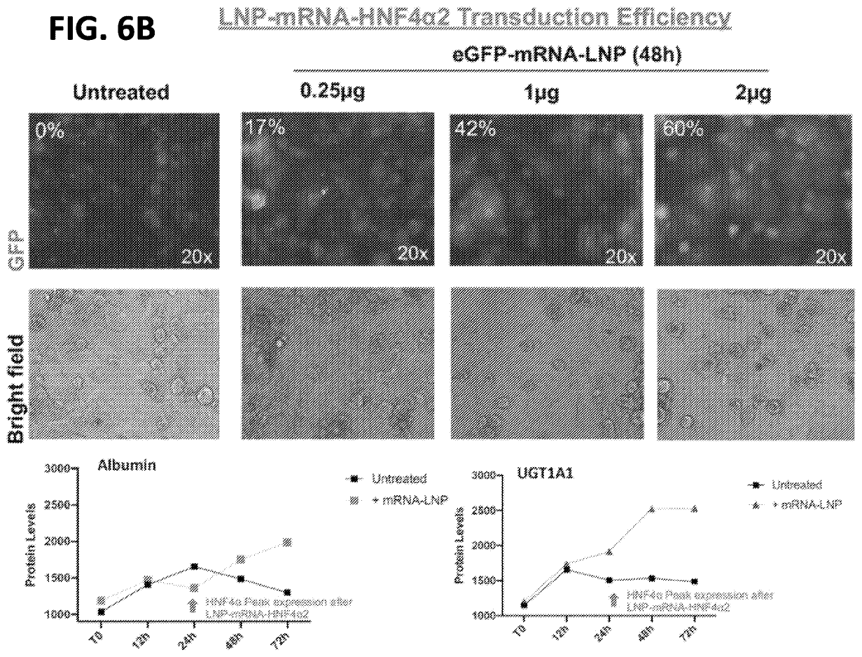

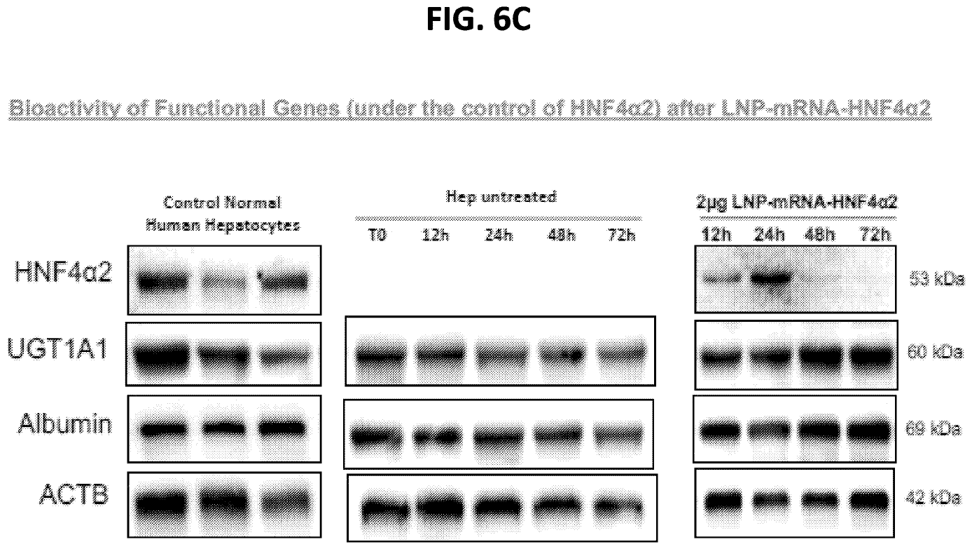

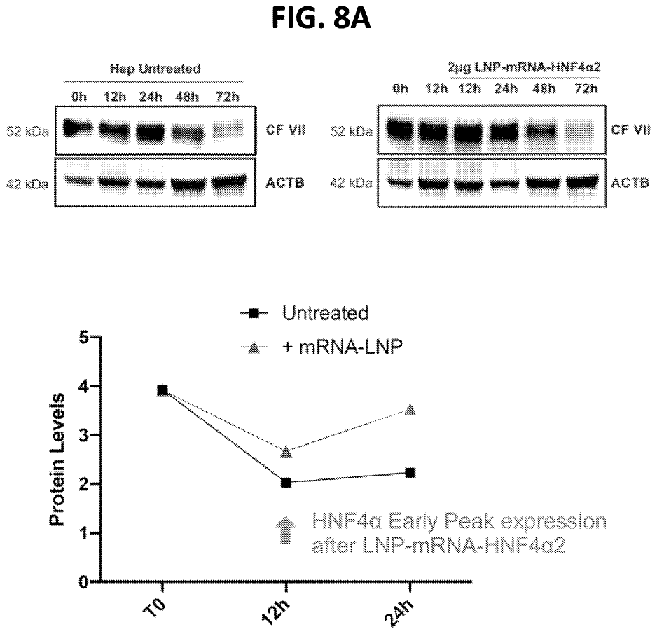

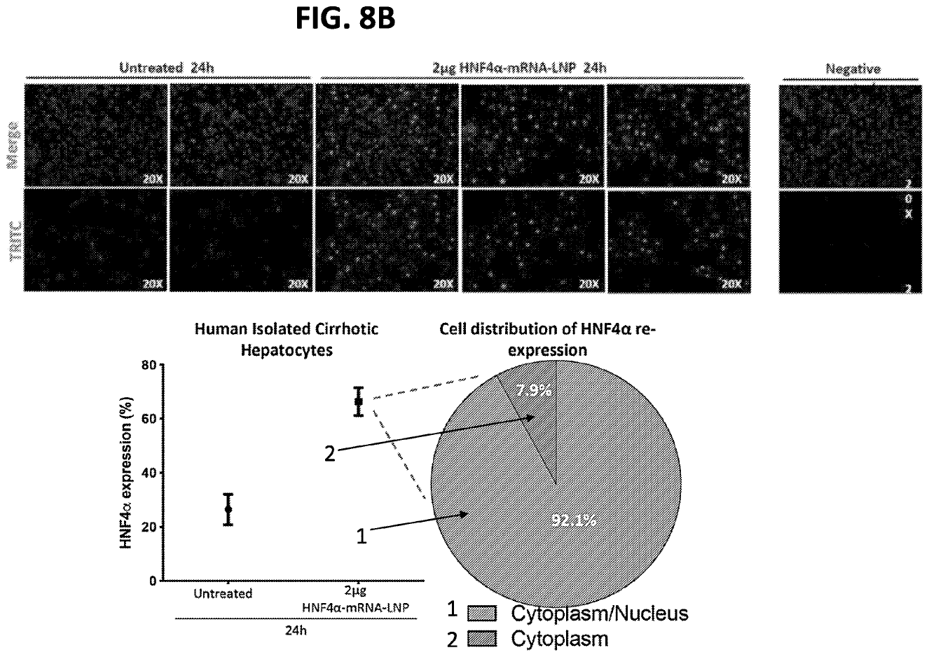

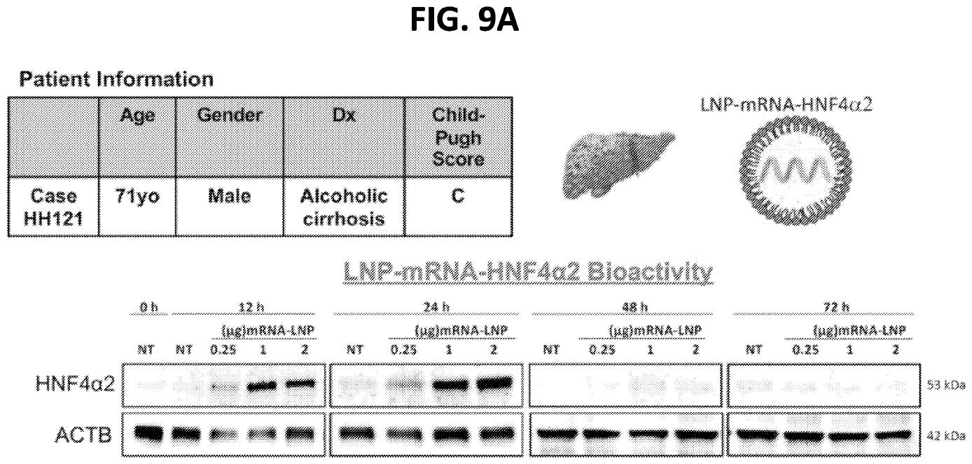

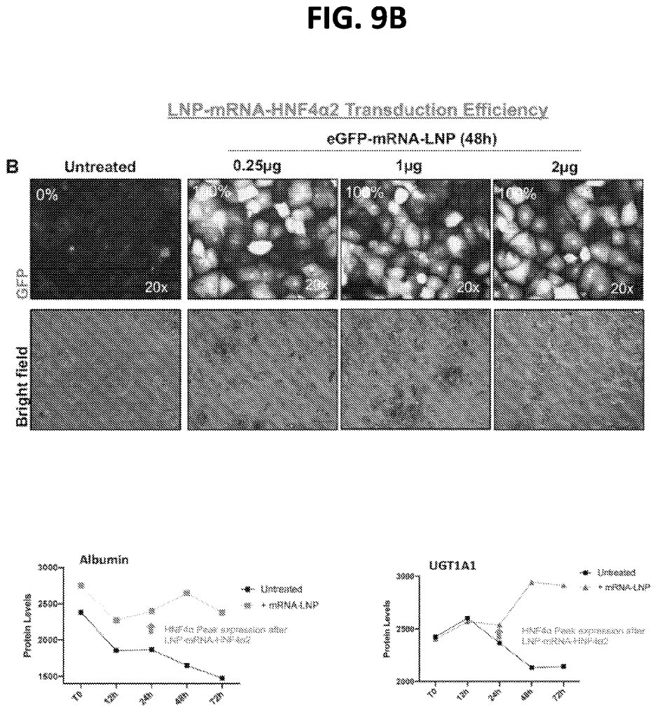

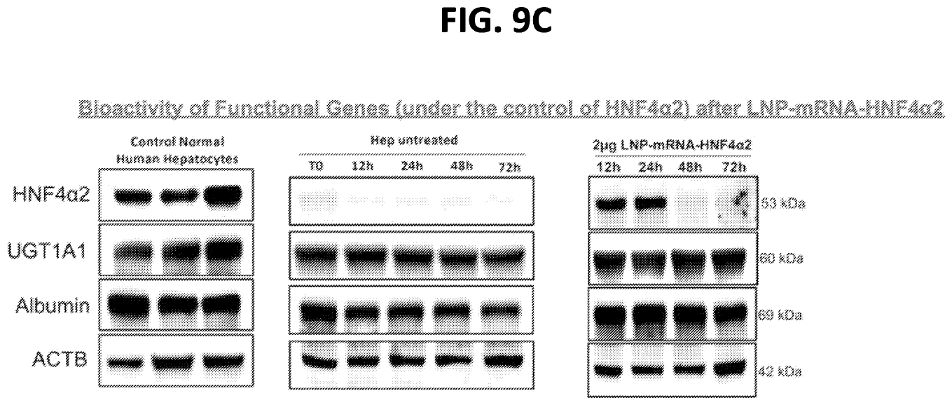

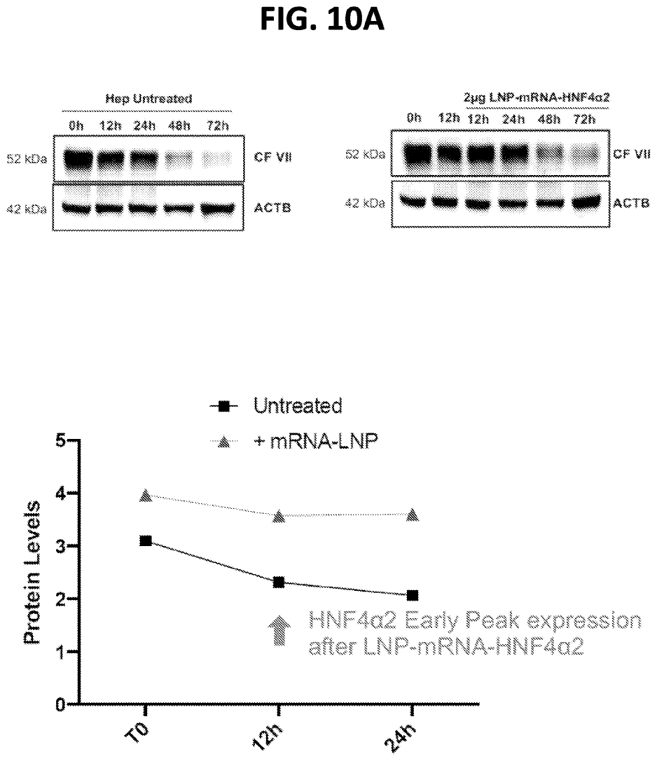

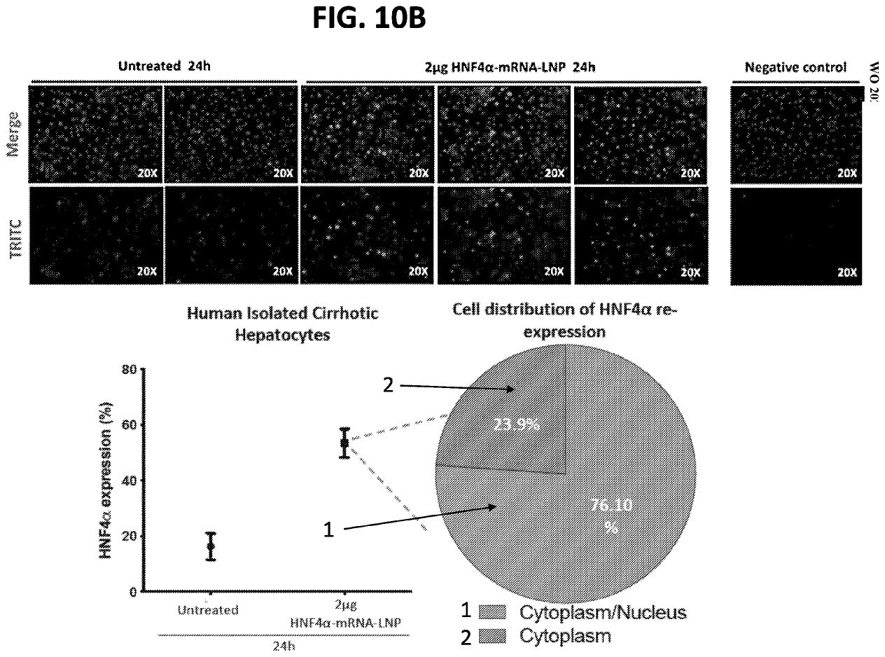

Expression and Subsequent Upregulation of Downstream Hepatocyte-Expressed Proteins After validating the delivery efficiency of LNP FM-1520A and the bioactivity of the mRNA-HNF4α2 construct, the ability of LNP FM-1520A carrying the mRNA-HNF4α2 construct (LNP-mRNA-HNF4α2) to upregulate HNF4α2 and downstream hepatocyte-expressed proteins in end-stage cirrhotic hepatocytes (FIG. 4) was tested. Primary human hepatocytes were isolated from cirrhotic livers of four patients who required liver transplantation for end-stage liver disease due to non-alcoholic steatohepatitis (NASH) or alcoholic cirrhosis. End-stage cirrhotic hepatocytes were treated with various doses of LNP-mRNA-HNF4α2 (0.25-2 μg), and the protein levels of HNF4α2, albumin, UDP glucuronosyltransferase 1A1 (UGT1A1), and coagulation factor VII (CF VII) were measured by Western blot 12-72 hours post treatment. End-stage cirrhotic hepatocytes treated with increasing doses of LNP-mRNA-HNF4α2 show increasing HNF4α2 protein levels starting at 12 hours and with peak expression at 24 hours after treatment (n=4) (FIGS. 5A-5C, 6A-6C, 7A-7C, 9A). Fluorescence imaging of end-stage cirrhotic hepatocytes treated with increasing doses of LNP-mRNA-eGFP shows increasing percentage of eGFP+ cells with maximal efficiency at a dose of 2 μg (n=4) (FIGS. 5A-5C, 6A-6C, 7A-7C, 9B). End-stage cirrhotic hepatocytes treated with 2 gg of LNP-mRNA-HNF4α2 show increased albumin and UGT1A1 protein levels at 48 and 72 hours after treatment (n=4) (FIGS. 5A-5C, 6A-6C, 7A-7C, 9C). In contrast, the levels of albumin and UGT1A1 in control untreated hepatocytes declined from 48 to 72 hours after treatment (FIGS. 5A-5C, 6A-6C, 7A-7C, 9C). End-stage cirrhotic hepatocytes treated with 2 ug of LNP-mRNA-HNF4α2 also exhibited increased CFVII protein levels starting at 12 hours and with peak expression at 24 hours after treatment (n=2) (FIGS. 8A-8B, 10A). In contrast, the levels of CF VII in control untreated hepatocytes declined from 12 to 24 hours after treatment (FIGS. 8A-8B, 10A).

### Example 6

### Treatment of End-Stage Cirrhotic Hepatocytes with LNP-mRNA-HNF4α2 Results in Increased Percentage of HNF4α2+ Cells

Previous studies have described downregulation of HNF4α2 and a mislocalization of HNF4α2 in the cytoplasm of hepatocytes in patients with end stage liver disease (Tafaleng et al., Hepatol Commun 5, 1911-1926 (2021); Florentino et al., Hepatol Commun 4, 859-875 (2020); Guzman-Lepe et al., Hepatol Commun 2, 582-594 (2018)). The effect of LNP-mRNA-HNF4α2 treatment on the expression and localization of HNF4α2 in end-stage cirrhotic hepatocytes was studied 24 hours after treatment. Immunofluorescence staining reveals an increased percentage of HNF4α2+ cells in LNP-mRNA-HNF4α2 treated hepatocytes (55-70%) compared to untreated controls (15-25%) (n=2) (FIGS. 8A-8B, 10B). Among LNP-mRNA-HNF4α2 treated hepatocytes expressing HNF4α2, 76-92% exhibited cytoplasmic and nuclear HNF4α2 localization while 8-24% exhibited purely cytoplasmic HNF4α2 localization (n=2) (FIGS. 8A-8B, 10B).

Taken together, the results evidence that LNP-mediated delivery of HNF4α2 mRNA into end-stage cirrhotic hepatocytes corrects the disrupted transcriptional network and normalizes the expression of proteins important for hepatocyte function. These changes can then lead to liver repair and improved liver function.

### Example 7

### Materials and Methods for Examples 8-9

Transfection with plasmid vectors for expressing P1-HNF4α or P2-HNF4α: Freshly isolated hepatocytes from livers of patients who underwent liver transplant for end-stage liver disease due to alcohol-mediated cirrhosis (Child-Pugh B) were washed and resuspended in HMM (Lonza, Walkersville, MD). The cells were transferred onto collagen-coated 12-well plates and transfected with either P1-HNF4α (RG217863, OriGene, Rockville, MD) or P2-HNF4α (RC238243, OriGene, Rockville, MD) plasmids at 1 μg using Lipofectamine 3000 (Thermo Fisher Scientific, Waltham, MA) according to manufacturer's instruction. The media was replaced with fresh HMM every day. Samples for RNA isolation, were collected 48 hours post-transfection.

Transduction with adeno-associated viral vectors for expressing HNF4α2: Freshly isolated hepatocytes from livers of patients who underwent liver transplant for end-stage liver disease due to non-alcoholic steatohepatitis (NASH) or alcohol-mediated cirrhosis (Child-Pugh C) were washed and resuspended in HCM (Lonza, Walkersville, MD). The cells were transferred onto collagen-coated 6-well plates and transduced with either AAV-10A2 or AAV-LK03 carrying either HNF4α2 or GFP (control) at an MOI of 1×10⁵. The media was replaced with fresh HCM every day. Samples for RNA isolation, protein lysate isolation, and 4% PFA fixation were collected 72 hours post-transduction.

Total RNA extraction and qPCR: RNA was extracted from primary human hepatocytes using the RNEASY® Mini Kit (Qiagen, Germantown, MD) according to manufacturer's instructions. cDNA was reverse transcribed from 0.5-1 μg total RNA using the SUPERSCRIPT® III cDNA synthesis kit (Invitrogen, Carlsbad, CA). For gene expression studies, reactions were performed on MicroAmp fast Optical 96-well plates (Applied Biosystems, Foster City, CA) using 1× TAQMAN® Fast Advanced Master Mix (Applied Biosystems, Foster City, CA), 1× TAQMAN® gene expression assays [Hs00604431_ml, Hs00609411_ml, Hs02511055_si, Hs01060665_g1] (Applied Biosystems, Foster City, CA), 100 ng of cDNA, and nuclease-free water (Invitrogen, Carlsbad, CA) to a final volume of 20 uL. qPCR was performed using the fast mode on an ABI StepOnePlus thermocycler (Applied Biosystems, Foster City, CA) set to auto-threshold and auto-background. All reactions were performed with two or three technical replicates with passive reference dye (ROX) normalization. Relative quantities were calculated using the delta-delta CT method using β-actin as the reference gene and normalizing to GFP-treated samples from the same patient. Values are reported as mean±SEM.

Western blot: Cells were incubated with RIPA lysis buffer (Sigma-Aldrich, Saint Louis, MO) containing 1× HALT™ Protease and Phosphatase Inhibitor Cocktail (Thermo Fisher Scientific, Waltham, MA) and incubated for 30 min at 4° C. Samples were centrifuged at 13,000×g for 10 min at 4° C. The supernatant from each sample was then transferred to a new microfuge tube and was used as the whole cell lysate. Protein concentrations were determined by comparison with a known concentration of bovine serum albumin using a Pierce BCA Protein Assay Kit (Thermo Fisher Scientific, Waltham, MA). About 30 μg of lysate were loaded per lane into 10% Mini-PROTEAN TGX gel (BioRad, Hercules, CA). After SDS-PAGE, proteins were transferred onto PVDF membrane (Thermo Fisher Scientific, Waltham, MA). Membranes were incubated with primary antibody solution [HNF4α: Abcam Ab41898, albumin: Bethyl Labs A80-229A, UGT1A1: Abcam Ab237810, β-actin: Cell Signaling 4970) overnight and then washed. Membranes were incubated for 1 hour in secondary antibody solution and then washed. Target antigens were finally detected using SuperSignal West Pico PLUS chemiluminescent substrate (Thermo Fisher Scientific, Waltham, MA). Images were scanned and analyzed using ImageJ software. All band density values were normalized to the band density for β-actin and are reported as mean±SEM.

Immunofluorescence Staining and Imaging: Primary human hepatocytes were washed with warm PBS, fixed with 4% PFA for 15 min, and washed 3× with PBS. Samples were washed 3× with wash buffer (PBS, 0.1% BSA, and 0.1% TWEEN 20) for 5 min and then blocked and permeabilized in blocking buffer (PBS, 10% normal donkey serum, 1% BSA, 0.1% TWEEN 20, and 0.1% Triton X-100) for 1 hour at room temperature. Samples were then incubated with primary antibody [HNF4α: Abcam Ab41898] in blocking buffer for 24 hours at 4° C. Samples were washed 3× with wash buffer for 5 min and incubated with secondary antibody in blocking buffer for 1 hour in the dark at room temperature. Samples were washed 3× with wash buffer for 5 min, 3× with PBS, and then counterstained with 1 μg/mL of Hoechst 33342 (Sigma, Saint Louis, MO) for 1 min at room temperature in the dark. Samples were washed 3× with PBS and stored in 2 mL PBS in the dark at 4° C. Samples were imaged using an Eclipse Ti inverted microscope (Nikon, Melville, NY) and the NIS-Elements software platform (Nikon, Melville, NY). For each sample, images were taken from three random fields using the 20× objective.

TABLE 4
TaqMan gene expression assay IDs used for qPCR

| Target | Gene Expression Assay | | |
|---|---|---|---|
| Gene | ID/Catalogue No. | Dye | Quencher |
| HNF4α2 | Hs00604431_m1 | FAM | MGB |
| Albumin | Hs00609411_m1 | FAM | MGB |
| CF VII | Hs01551992_m1 | FAM | MGB |
| UGT1A1 | Hs02511055_s1 | FAM | MGB |
| ACTB | Hs01060665_g1 | FAM | MGB |

TABLE 5
Antibodies and dilutions used for western
blot and immunofluorescence staining.

| | | | | |
|---|---|---|---|---|
| | | | Catalogue | |
| Antibody | Host | Company | No. | Dilution |

Western Blot

| | | | | |
|---|---|---|---|---|
| anti-P1-HNF4α | mouse | Abcam | Ab41898 | 1:1000 |
| anti-P2-HNF4α | mouse | R&D Systems | PP-H6939-00 | 1:500 |
| anti-UGT1A1 | rabbit | Abcam | Ab237810 | 1:2000 |
| anti-albumin | goat | Bethyl | A80-229A | 1:1000 |
| anti-ACTB | rabbit | Cell Signaling Tech | 4970 | 1:1000 |
| anti-GAPDH | mouse | Proteintech | 60004-1-Ig | 1:10000 |

Immunohistochemistry and Immunofluorescence staining

| | | | | |
|---|---|---|---|---|
| anti-HNF4α | mouse | Abcam | Ab41898 | 1:1000 |

### Example 8

### P1-HNF4α Isoform 2 has Transcriptional Capabilities to Modulate the Expression of Hepatocyte-Specific Genes Related to Critical Functions and -Adeno-Associated Virus (AAV) is not and Effective Method for the Re-Expression of Hepatocyte-Specific Genes Downstream of P1-HNF4α Isoform 2

Several studies have described successful transgene delivery in healthy primary human hepatocytes or humanized mouse livers using AAVs, however, equivalent studies in primary human hepatocytes isolated from decompensated cirrhotic livers have not been reported. Our past experience with the introduction of exogenous genes into rodents using AAVs was successful. Nonetheless, here we evaluated first the transcriptional capabilities of different isoforms of HNF4α and also the capacity of AAVs to target the transcription and translation of downstream proteins controlled by P1-HNF4α isoform 2.

### Evaluation of P1-/P2-HNF4α Expression in Primary Human Hepatocytes Isolated from Explanted Liver with Cirrhosis and End-Stage Liver Disease

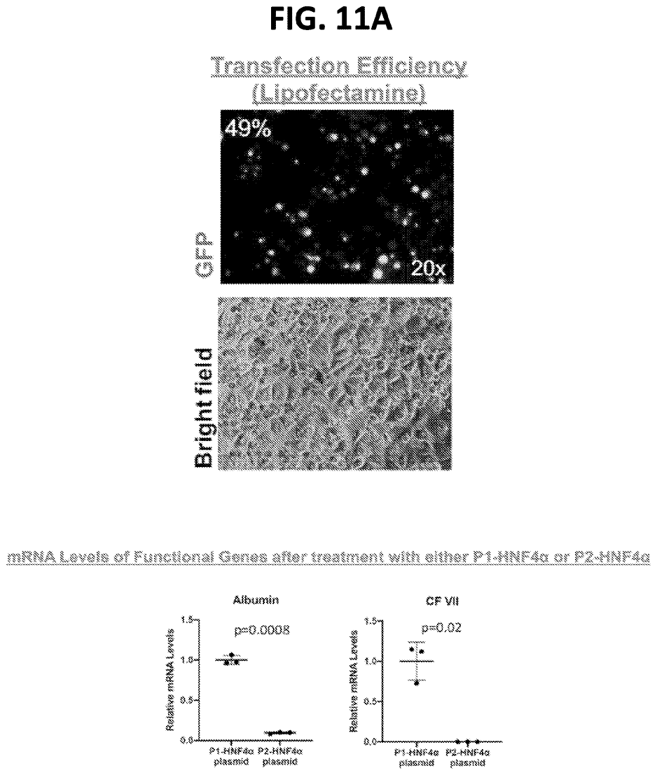

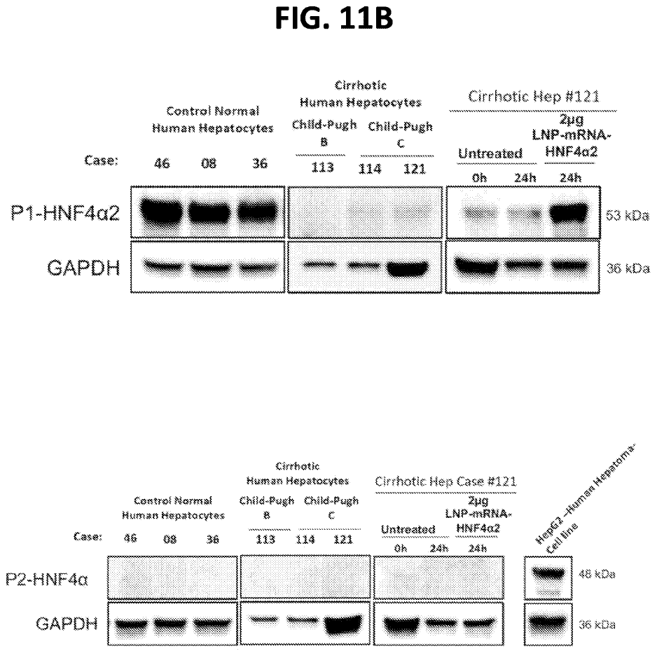

HNF4α has 12 isoforms generated through two promoters (P1 and P2) and alternative splicing. Each promoter could have different transcriptional capabilities and the HNF4α isoforms can be expressed and function differently depending on the state of differentiation of hepatocytes, developmental stage and disease state. In order to understand which HNF4α isoform could control the expression and function of downstream hepatocyte-specific genes (albumin and coagulation factor VII), cirrhotic human hepatocytes that were freshly isolated from patients explanted livers with End-Stage Liver Disease (ESLD) were treated with either P1-HNF4α or P2-HNF4α. About 49% of the cirrhotic human hepatocytes were transduced using lipofectamine and only overexpression of P1-HNF4α was able to induce and increased in the downstream hepatocyte-specific functional genes (albumin and coagulation factor VII). These results were unexpected and demonstrate that the transcriptional capabilities of HNF4α isoforms vary in different states of human hepatocyte differentiation or disease (FIG. 11A).

To test which promoter isoforms are expressed in normal human hepatocytes or cirrhotic human hepatocytes with end-stage liver disease (ESLD), the protein expression of P1-HNF4α and P2-HNF4α was analyzed. It was found that both P1 and P2 isoforms are downregulated in disease human hepatocytes (cirrhosis and ESLD). However, only P1-HNF4α is strongly expressed in normal human hepatocytes. The expression of P1-HNF4α and P2-HNF4α in cirrhotic hepatocytes with ESLD, treated with LNP-mRNA-HNF4α2, was analyzed. It was observed that specifically P1-HNF4α expression and function was rescued by the treatment. As expected P2-HNF4α expression was present in human hepatoma cells. These results indicated that P1-HNF4α expression, specifically HNF4α2 is responsible for the up-regulation and function of protein levels of downstream hepatocyte-specific genes (FIGS. 11A-11B).

Treatment of end-stage cirrhotic hepatocytes with AAV-10A2-HNF4α2 and AAV-LK03-HNF4α2 leads to upregulation of HNF4α but was highly variable between patients: Primary human hepatocytes were isolated from cirrhotic livers of three patients who required liver transplantation for end-stage liver disease due to non-alcoholic steatohepatitis (NASH) or alcoholic cirrhosis (Child-Pugh C). End-stage cirrhotic hepatocytes were transduced with AAV vectors (serotypes 10A2 or LK03) for expressing either HNF4α2 or GFP (control).

qPCR and Western blot analysis at 72 hours after transduction revealed an increase in HNF4α mRNA and protein levels in cells treated with AAV vectors expressing HNF4α2 compared to cells that received the GFP control vectors. Immunofluorescence staining for HNF4α in transduced cells confirmed this increase in HNF4α protein levels. There was a marginally higher upregulation of HNF4α in cells treated with the AAV-10A2 serotype compared to the AAV-LK03. However, there was considerable variability in the observed upregulation of HNF4α between patient samples as shown by the spread of patient data points.

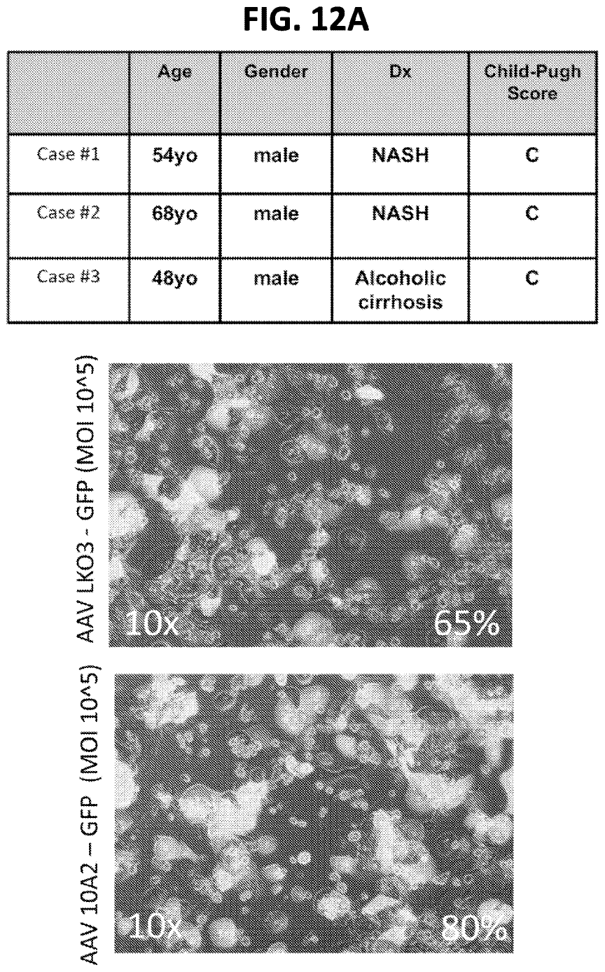

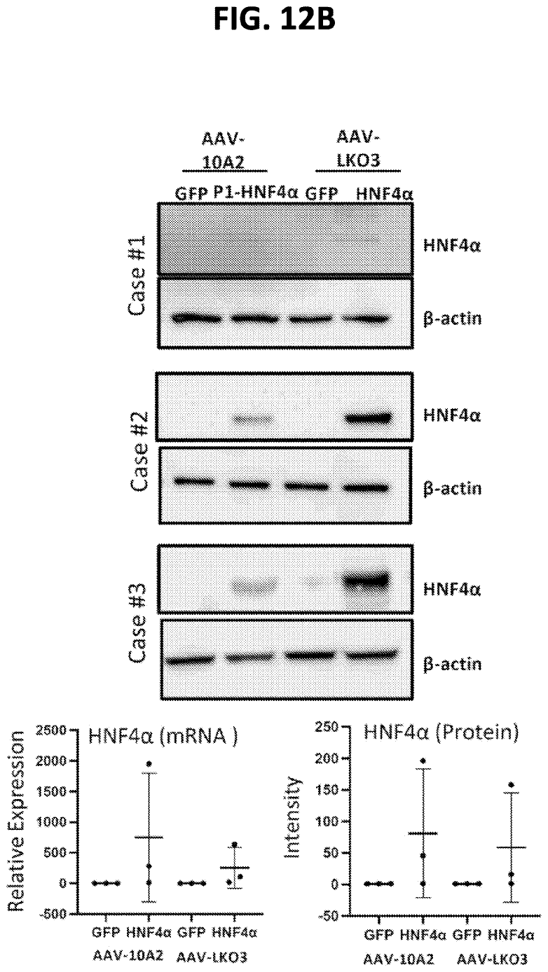

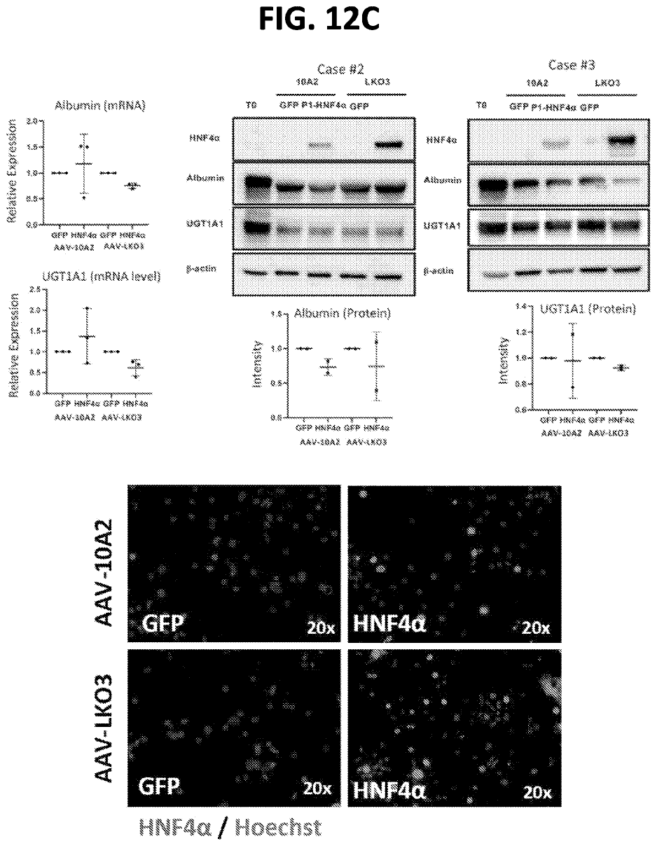

Analysis of genes important for hepatocyte function showed an increase in the mRNA levels of albumin and UDP glucuronosyltransferase 1A1 (UGT1A1) in cells treated with AAV-10A2-HNF4α2, but not in those that received AAV-LK03-HNF4α2, compared to controls (FIGS. 12A-12C). However, albumin and UGT1A1 protein levels were not increased in cells treated with AAV vectors expressing HNF4α2 compared to cells that received the GFP control vectors. These results are unexpected due to the current development of gene transfer with AAV vectors in clinical trials for genetic liver diseases and our previous experience in animal (rodent) models.

### Example 9

### LPN-mRNA-HNF4α2 Induce the Protein Expression of Functional Downstream Genes (Under the Control of HNF4α) Albumin and UGT1A1

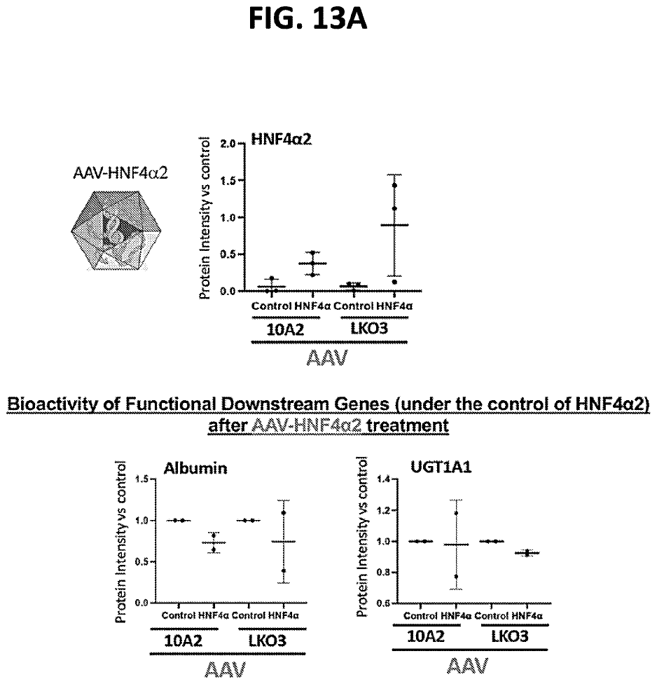

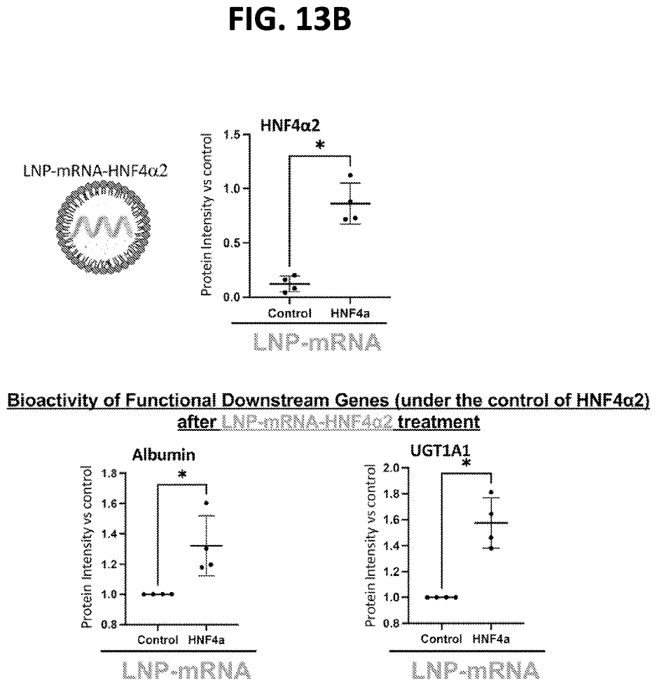

HNF4α is the master transcription factor in the liver, it stabilizes the rest of the hepatic transcriptional network to ensure proper hepatocyte differentiation and function. Several studies have described successful transgene delivery in healthy primary human hepatocytes or humanized mouse livers using AAVs, however, equivalent studies in primary human hepatocytes isolated from decompensated cirrhotic livers have not been reported. Previously, the introduction of exogenous genes into rodents using AAVs was successful. Nonetheless, the transcriptional capabilities of AAVs and LNP-mRNA to increase the controlled expression of functional downstream genes (under the control of HNF4α) Albumin and UGT1A1 was evaluated (FIGS. 13A-13B).

### Treatment of End-Stage Cirrhotic Hepatocytes with AAV-10A2-HNF4α2 or AAV-LK03-HNF4α2 Leads to Upregulation of HNF4α but was Highly Variable Between Patients

Primary human hepatocytes were isolated from cirrhotic livers of four patients who required liver transplantation for end-stage liver disease due to non-alcoholic steatohepatitis (NASH) or alcoholic cirrhosis (Child-Pugh B and C). End-stage cirrhotic hepatocytes were transduced with AAV vectors (serotypes 10A2 or LK03) for expressing HNF4α2 (n=3) or LNP-mRNA-HNF4α2 (n=4). Quantification of Western blot analysis after transduction with AAVs revealed an increase in HNF4α protein levels in cells treated with AAV vectors expressing HNF4α2 compared to control cells. There was considerable variability in the observed upregulation of HNF4α between patient samples as shown by the spread of patient data points for both AAV serotypes. However, when treated with LNP-mRNA-HNF4α2 all cases showed stable increased expression of HNF4α2.

Protein analysis of genes important for hepatocyte function (albumin and UDP glucuronosyltransferase 1A1; UGT1A1) revealed that there was not increased in all cases treated with AAV vectors expressing HNF4α2 compared to control cells (FIG. 16). Surprisingly, in the other hand, all cases treated with LNP-mRNA-HNF4α2 technology showed and impressive increased in protein expression of both human albumin and UGT1A1.

### Example 10

### Evaluation of P1-/P2-HNF4α Expression in Primary Human Hepatocytes Isolated from Explanted Liver with Cirrhosis and End-Stage Liver Disease

It was determined that P1HNF4α protein is predominantly express in primary human hepatocytes and the P1-HNF4α protein expression is diminished in human hepatocytes from cirrhotic livers with end-stage liver disease. Modified mRNA for P1-HNF4 α 2 was more effective than viral transduction of the HNF4 α transgene to not only upregulate HNF4α but also downstream functional genes. A superior optimized modified mRNA-HNF4 α 2 was produced that was unexpected superior (Hepatol Commun. 2021 November; 5(11):1911-1926). Based on this information, optimized sequences were produced for both mRNA-HNF4α 1 and mRNA-HNF4 α 2.

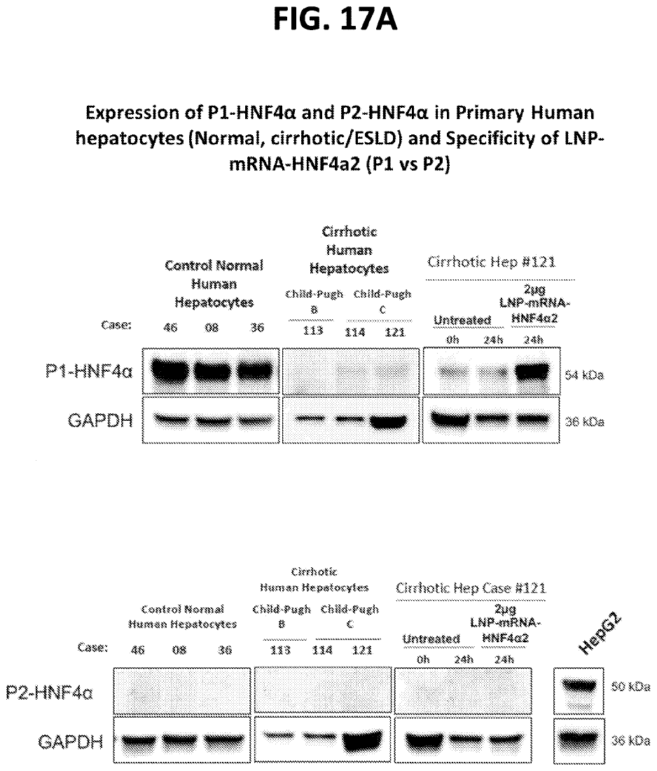

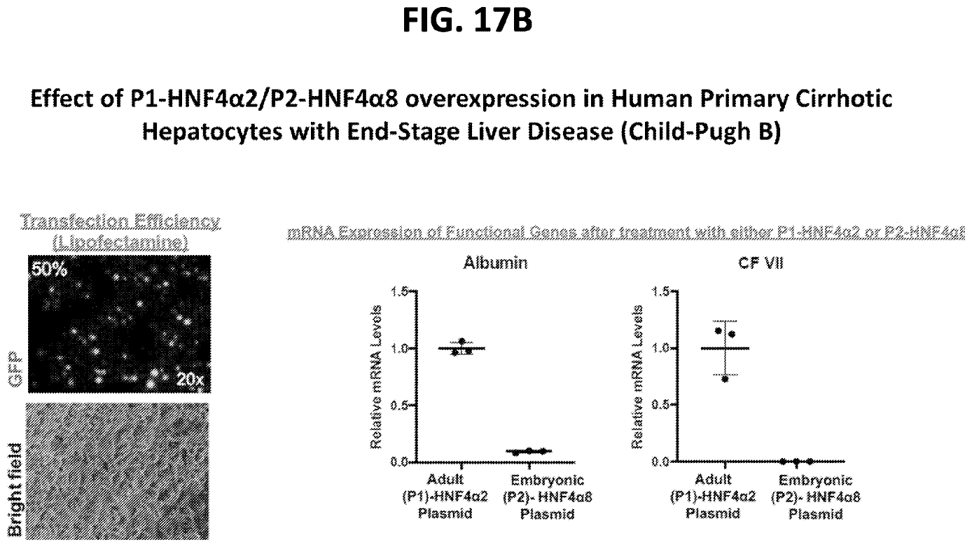

FIG. 17 shows an evaluation of P1-/P2-HNF4α Expression in Primary Human Hepatocytes isolated from Explanted liver with Cirrhosis and End-Stage Liver Disease. As discussed above, HNF4α has 12 isoforms generated through two promoters (P1 and P2) and alternative splicing. Each promoter could have different transcriptional capabilities and the HNF4α isoforms can be expressed and function differently depending on the state of differentiation of hepatocytes, developmental stage and disease state. In order to determine which HNF4α isoform could control the expression and function of downstream hepatocyte-specific genes (albumin and coagulation factor VII), cirrhotic human hepatocytes that were freshly isolated from patients explanted livers with End-Stage Liver Disease (ESLD) were treated with either P1-HNF4α or P2-HNF4α. About half of the cirrhotic human hepatocytes were transduced using lipofectamine, and only overexpression of P1-HNF4α was able to induce and increased in the downstream hepatocyte-specific functional genes (albumin and coagulation factor VII). These results were unexpected and demonstrate that the transcriptional capabilities of HNF4α isoforms vary in different states of human hepatocyte differentiation or disease.

To test which promoter isoforms were expressed in normal human hepatocytes or cirrhotic human hepatocytes with end-stage liver disease (ESLD), the protein expression of P1-HNF4α and P2-HNF4α was analyzed. It was found that both P1 and P2 isoforms are downregulated in disease human hepatocytes (cirrhosis and ESLD). However, only P1-HNF4α is strongly expressed in normal human hepatocytes. The expression of P1-HNF4α and P2-HNF4α was analyzed in cirrhotic hepatocytes with ESLD treated with LNP-mRNA-HNF4α2. It was observed that specifically P1-HNF4α expression and function was rescued by the treatment. As expected P2-HNF4α expression was present in human hepatoma cells.

These results indicate that P1-HNF4α expression, specifically HNF4α2 is responsible for the up-regulation and function of protein levels of downstream hepatocyte-specific genes (FIG. 17A). In this study, expression of the Adult P1-HNF4α2 isoform led to a significant upregulation of HNF4α target genes that are important in mature hepatocyte function (FIG. 17B), whereas similar expression of the embryonic (P2) HNF4α8 did not. Plasmids encoding either adult P1-HNF4α2 or embryonic (P2) HNF4α8 were transfected into cirrhotic human primary hepatocytes with end-stage liver disease. Only the adult P1-HNF4α2 lead to increased expression of downstream genes Albumin and CFVII 48 hours after transfection (FIG. 17B).

### Example 11

### Treatment of End-Stage Cirrhotic Hepatocytes with LNP-mRNA-HNF4α2 is More Effective to Upregulate P1-HNF4α and Downstream Functional Genes than AAV-10A2-HNF4α2 and AAV-LK03-HNF4α2

Primary human hepatocytes were isolated from cirrhotic livers of three patients who required liver transplantation for end-stage liver disease due to non-alcoholic steatohepatitis (NASH) or alcoholic cirrhosis (Child-Pugh C). End-stage cirrhotic hepatocytes were transduced with AAV vectors (serotypes 10A2 or LK03) for expressing either HNF4α2 or GFP (control).

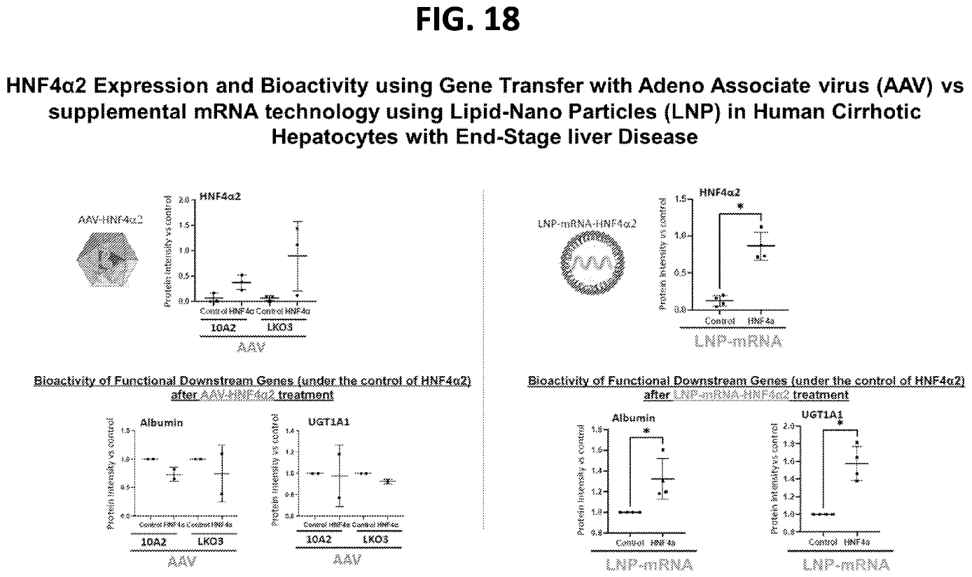

Western blot analysis at 72 hours after transduction revealed an increase in HNF4α mRNA and protein levels in cells treated with AAV vectors expressing HNF4α2 compared to cells that received the GFP control vectors. Immunofluorescence staining for HNF4α in transduced cells confirmed this increase in HNF4α protein levels. There was a marginally higher upregulation of HNF4α in cells treated with the AAV-10A2 serotype compared to the AAV-LK03. However, there was considerable variability in the observed upregulation of HNF4α between patient samples as shown by the spread of patient data points (FIG. 18A).

Analysis of genes important for hepatocyte function showed an increase in the mRNA levels of albumin and UDP glucuronosyltransferase 1A1 (UGT1A1) in cells treated with AAV-10A2-HNF4α2, but not in those that received AAV-LK03-HNF4α2, compared to controls. However, albumin and UGT1A1 protein levels were not increased in cells treated with AAV vectors expressing HNF4α2 compared to cells that received the GFP control vectors (FIG. 18A).

In contrast, modified mRNA-HNF4α2 construct (LNP-mRNA-HNF4α2) was highly effective for upregulating HNF4α2 and downstream hepatocyte-expressed proteins (albumin, UDP glucuronosyltransferase 1A1 (UGT1A1)] in end-stage cirrhotic hepatocytes, see FIG. 18B). Primary human hepatocytes were isolated from cirrhotic livers of four patients who required liver transplantation for end-stage liver disease due to non-alcoholic steatohepatitis (NASH) or alcoholic cirrhosis for these experiments.

### Example 12

### Production of a Superior Optimized Modified mRNA-HNF4α2

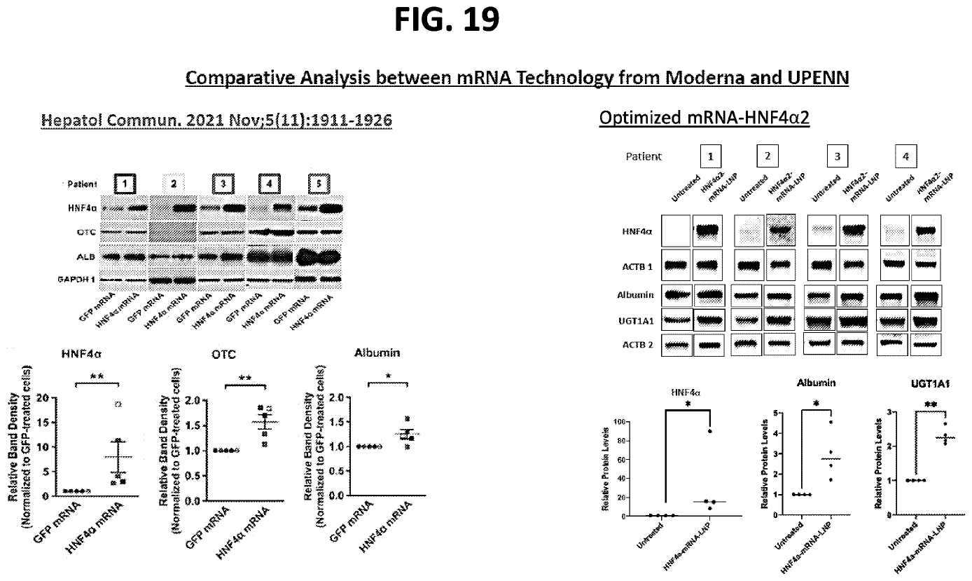

The ability of published modified mRNA-HNF4α2 was compared to the presently disclosed optimized sequence of modified mRNA-HNF4α2 to induce protein in human cirrhotic hepatocytes (FIG. 19). Western blot analysis also revealed a significant increase in protein expression after re-expression of HNF4α2 in primary decompensated cirrhotic hepatocytes. On average, there was a 70% increase in P1-HNF4α, a 25% increase in albumin, and a 60% increase in OTC protein expression 24 hours after HNF4α2 re-expression in decompensated cirrhotic hepatocytes while the presently disclosed optimized modified mRNA-HNF4α2 (SEQ ID NO: 19) induced 3225% increase in P1-HNF4α, a 170% in albumin, and a 200% increase in UGT1A1 protein expression 24 hours after HNF4α2 re-expression.

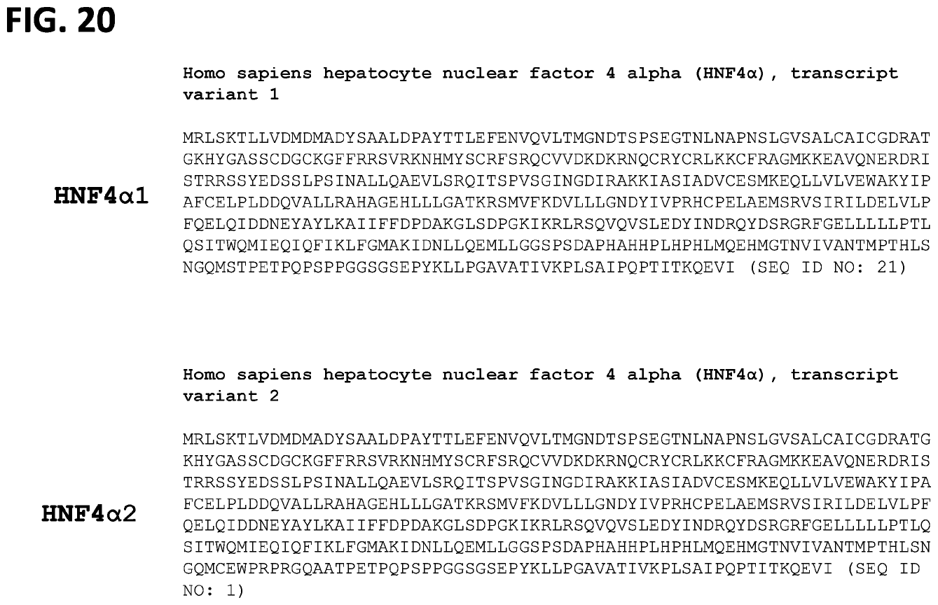

P1-HNF4α isoform expression is lower in end-stage liver disease (ESLD). The overwhelming majority of HNF4α protein expressed in liver is derived from the P1 promoter which produces the adult HNF4α1, HNF4α2 and to a lesser extent HNF4α3. HNF4α2 is the predominant mRNA isoform in liver. The ratio has been estimated by RT-PCR in rodent hepatocytes (40%-a1:50%-a2:10% α3). Expression of these isoforms is lost along with liver function in end-stage liver disease. It is quite difficult to distinguish α1 and a2 isoform proteins by Western blot with, as there is only a 10/465 aa difference in the amino acid sequence (FIG. 20). P1-HNF4α isoforms 4, 5, and 6 are not normally expressed in hepatocytes (Harris et al., Diabetes 57, 1745-1752 (2008)) and do not bind DNA (Lambert et al., Mol Cell Proteomics 19, 808-827 (2020)).

Isoform 3, when expressed, shows weaker if any target gene activation (Lambert et al. Mol Cell Proteomics 19, 808-827 (2020); Suaud et al., Biochem J 340 (Pt 1), 161-169 (1999)). Isoforms 1 and 2 are the best candidates for therapeutic purposes. The design of modified mRNA was optimized for both isoforms, that differ from naïve mRNA sequences (coding mRNA sequences) (FIGS. 20 and 21).

### Example 13

### Materials and Methods for Examples 10-12

Transduction with adeno-associated viral vectors for expressing HNF4α2: Freshly isolated hepatocytes from livers of patients who underwent liver transplant for end-stage liver disease due to non-alcoholic steatohepatitis (NASH) or alcohol-mediated cirrhosis (Child-Pugh C) were washed and resuspended in HCM (Lonza, Walkersville, MD). The cells were transferred onto collagen-coated 6-well plates and transduced with either AAV-10A2 or AAV-LK03 carrying either HNF4α2 or GFP (control) at an MOI of 1×10⁵. The media was replaced with fresh HCM every day. Samples for RNA isolation, protein lysate isolation, and 4% PFA fixation were collected 72 hours post-transduction.

Western blot: Cells were incubated with RIPA lysis buffer (Sigma-Aldrich, Saint Louis, MO) containing 1× HALT™ Protease and Phosphatase Inhibitor Cocktail (Thermo Fisher Scientific, Waltham, MA) and incubated for 30 min at 4° C. Samples were centrifuged at 13,000×g for 10 min at 4° C. The supernatant from each sample was then transferred to a new microfuge tube and was used as the whole cell lysate. Protein concentrations were determined by comparison with a known concentration of bovine serum albumin using a Pierce BCA Protein Assay Kit (Thermo Fisher Scientific, Waltham, MA). About 30 μg of lysate were loaded per lane into 10% Mini-PROTEAN TGX gel (BioRad, Hercules, CA). After SDS-PAGE, proteins were transferred onto PVDF membrane (Thermo Fisher Scientific, Waltham, MA). Membranes were incubated with primary antibody solution [HNF4α: Abcam Ab41898, albumin: Bethyl Labs A80-229A, UGT1A1: Abcam Ab237810, β-actin: Cell Signaling 4970) overnight and then washed. Membranes were incubated for 1 hour in secondary antibody solution and then washed. Target antigens were finally detected using SuperSignal West Pico PLUS chemiluminescent substrate (Thermo Fisher Scientific, Waltham, MA). Images were scanned and analyzed using ImageJ software. All band density values were normalized to the band density for β-actin and are reported as mean±SEM.

TABLE 6
Antibodies and dilutions used for Western blot

| | | | Catalogue | |
|---|---|---|---|---|
| Antibody | Host | Company | No. | Dilution |
| anti-P1-HNF4α | mouse | Abcam | Ab41898 | 1:1000 |
| anti-albumin | goat | Bethyl | A80-229A | 1:1000 |
| anti-UGT1A1 | rabbit | Abcam | Ab237810 | 1:2000 |
| anti-CF VII | rabbit | Abcam | Ab151543 | 1:1000 |
| anti-OTC | mouse | Abcam | Ab180401 | 1:1000 |
| anti- P2-HNF4α | rabbit | R&D | H6939-00 | 1:1000 |
| anti-ACTB | rabbit | Cell Signaling | 4970 | 1:1000 |
| | | Tech | | |
| anti-GAPDH | mouse | Proteintech | 60004-1-Ig | 1:10000 |
| anti-mouse IgG-HRP | goat | EMD Millipore | 12-349 | 1:10000 |
| anti-rabbit IgG- | goat | Thermo Fisher | 31460 | 1:20000 |
| HRP | | Scientific | | |
| anti-goat IgG-HRP | donkey | Thermo Fisher | A-1105 | 1:10000 |
| | | Scientific | | |

Production of the mRNA encoding the human hepatocyte nuclear factor 4α variant 2 (mRNA-HNF4α2): Codon optimized human hepatocyte nuclear factor 4α variant 2 (human HNF4α2) was cloned downstream of a Kozak sequence into an mRNA production plasmid (optimized 3′ and 5′ UTR and containing a 101 polyA tail), in vitro transcribed in the presence of the presence of N1-methylpseudouridine modified nucleoside (N1mψ), co-transcriptionally capped using the CLEANCAP™ technology (TriLink) and cellulose purified to remove dsRNA. Purified mRNA was ethanol precipitated, washed, resuspended in nuclease-free water, and subjected to quality control (electrophoresis, dot blot, and transfection into human DCs). mRNA was stored at −20° C. until use.

It will be apparent that the precise details of the methods or compositions described may be varied or modified without departing from the spirit of the described aspects of the disclosure. We claim all such modifications and variations that fall within the scope and spirit of the claims below.

1. A recombinant mRNA encoding an HNF4α1 or HNF4α2, wherein the recombinant mRNA comprises:
i) a nucleic acid sequence at least 95% identical to one of
a) SEQ ID NO: 2 [HNF4α2],
b) SEQ ID NO: 22 [HNF4α1]; or
c) SEQ ID NO: 30 [HNF4α1];
and
ii) optionally comprises one or more of:
d) a 5′ capping structure,
e) a promoter,
f) a nucleic acid molecule encoding a signal peptide,
g) a 5′ untranslated region (UTR),
h) a 3′ UTR, and
i) a polyA tail.

2. The recombinant mRNA of claim 1, comprising:
a nucleic acid sequence at least 95% identical to SEQ ID NO: 2 that encodes SEQ ID NO: 1;
a nucleic acid sequence at least 95% identical to SEQ ID NO: 22 that encodes SEQ ID NO: 21; or
a nucleic acid sequence at least 95% identical to SEQ ID NO: 30 that encodes SEQ ID NO: 21.

3. The recombinant mRNA of claim 1, wherein the recombinant mRNA comprises SEQ ID NO: 2, SEQ ID NO: 22, or SEQ ID NO: 30.

4. The recombinant mRNA of claim 1, wherein the recombinant mRNA comprises the 5′UTR, and wherein the 5′ UTR comprises the nucleic acid sequence of one of SEQ ID NOs: 8-10 and 24.

5. The recombinant mRNA of claim 1, wherein the recombinant mRNA comprises the 3′ UTR, and wherein the 3′ UTR comprises the nucleic acid sequence of one of SEQ ID NOs: 11-17 and 25.

6. The recombinant mRNA of any one of claim 1, comprising the nucleic acid molecule encoding the signal peptide, wherein the signal peptide comprises the amino acid sequence of one of SEQ ID NOs: 3-7.

7. The recombinant mRNA of claim 1, comprising a nucleic acid sequence set forth as any one of SEQ ID NOs: 18, 20, or 23.

8. The recombinant mRNA of claim 1, comprising the 5′ capping structure, that is a 7mG(5′)ppp(5′)N1mpNp cap at the 5′ end of the 5′UTR.

9. The recombinant mRNA of claim 1, wherein the recombinant mRNA comprises a modified nucleotide that increases half-life of the recombinant mRNA, increases protein production, and/or reduces induction of innate immunity.

10. The recombinant mRNA of claim 9, wherein the modified nucleotide is modified uridine or a pseudouridine.

11. The recombinant mRNA of claim 10, wherein the pseudouridine is 1-methylpseudouridine.

12. A lipid nanoparticle comprising the recombinant mRNA of claim 1.

13. The lipid nanoparticle of claim 12, comprising a polyethylene glycol (PEG)-modified lipid, a non-cationic lipid, a sterol, an ionizable cationic lipid, or any combination thereof.

14. The lipid nanoparticle of claim 13, wherein the PEG has an average molecular weight of about 20000 (PEG2000 DMG).

15. The lipid nanoparticle of claim 12, wherein the lipid nanoparticle comprises the ionizable lipid.

16. The lipid nanoparticle of claim 12, wherein the lipid nanoparticle comprises (2 hydroxyethyl)(6 oxo 6-(undecycloxy)hexyl)amino)octanoate, 1,2 distearoyl sn glycerol-3 phosphocholine (DSPC), cholesterol, and 1-monomethoxypolyethyleneglycol-2,3,dimyristylglycerol.

17. A method for treating liver disease or end-stage liver failure in a subject, comprising:
administering to the subject a therapeutically effective amount of a recombinant mRNA encoding HNF4α1,
thereby treating the liver disease or end-stage liver failure in the subject.

18. The method of claim 17, wherein the method increases serum albumin, decreases serum ammonia, improve coagulation activity, decreases ascites production, improves neuropsychological status, decreases bilirubin, improves apolipoprotein and/or improves portal vein or systemic blood flow clearance of cholate in the subject.

19. The method of claim 17, wherein the subject is human.

20-34. (canceled)

35. The method of claim 17, comprising administering to the subject a therapeutically effective amount of a lipid nanoparticle comprising the recombinant mRNA.

36-39. (canceled)

40. The method of claim 17, comprising administering to the subject a composition comprising the therapeutically effective amount of the recombinant mRNA and a pharmaceutically acceptable carrier.

41-44. (canceled)

45. The method of claim 17, wherein the liver disease is a degenerative liver disease.

46. The method of claim 45, wherein the degenerative liver disease is non-alcoholic fatty liver disease (NASH) or alcohol related liver disease (ALD).

47. The method of claim 17, wherein the subject has liver disease with a Child-Pugh score level of A, B or C.

48. A method for treating end-stage liver disease in a subject, comprising:
administering to the subject a therapeutically effective amount of a recombinant mRNA encoding HNF4α2,
thereby treating the end-stage liver disease in the subject.

49. The method of claim 48, wherein the method increases serum albumin, decreases serum ammonia, improve coagulation activity, decreases ascites production, improves neuropsychological status, decreases bilirubin, improves apolipoprotein and/or improves portal vein or systemic blood flow clearance of cholate in the subject.

50. The method of claim 48, wherein the subject is human.

51-74. (canceled)


SEQUENCE LISTING (33 sequences)

SEQ ID NO: 1 - protein, 474 aa, Homo sapiens
MRLSKTLVDMDMADYSAALDPAYTTLEFENVQVLTMGNDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMCEWPRPRGQAATPETPQPSPPGGSGSEPYKLLPGAVATIVKPLSAIPQPTITKQEVI

SEQ ID NO: 2 - RNA, 1425 nt, Homo sapiens
  source 1..1425: mol_type = mRNA
atgcgcctgtccaagaccctggtggacatggacatggccgactactccgccgccctggaccccgcctacaccaccctggagttcgagaacgtgcaggtgctgaccatgggcaacgacacctccccctccgagggcaccaacctgaacgcccccaactccctgggcgtgtccgccctgtgcgccatctgcggcgaccgcgccaccggcaagcactacggcgcctcctcctgcgacggctgcaagggcttcttccgccgctccgtgcgcaagaaccacatgtactcctgccgcttctcccgccagtgcgtggtggacaaggacaagcgcaaccagtgccgctactgccgcctgaagaagtgcttccgcgccggcatgaagaaggaggccgtgcagaacgagcgcgaccgcatctccacccgccgctcctcctacgaggactcctccctgccctccatcaacgccctgctgcaggccgaggtgctgtcccgccagatcacctcccccgtgtccggcatcaacggcgacatccgcgccaagaagatcgcctccatcgccgacgtgtgcgagtccatgaaggagcagctgctggtgctggtggagtgggccaagtacatccccgccttctgcgagctgcccctggacgaccaggtggccctgctgcgcgcccacgccggcgagcacctgctgctgggcgccaccaagcgctccatggtgttcaaggacgtgctgctgctgggcaacgactacatcgtgccccgccactgccccgagctggccgagatgtcccgcgtgtccatccgcatcctggacgagctggtgctgcccttccaggagctgcagatcgacgacaacgagtacgcctacctgaaggccatcatcttcttcgaccccgacgccaagggcctgtccgaccccggcaagatcaagcgcctgcgctcccaggtgcaggtgtccctggaggactacatcaacgaccgccagtacgactcccgcggccgcttcggcgagctgctgctgctgctgcccaccctgcagtccatcacctggcagatgatcgagcagatccagttcatcaagctgttcggcatggccaagatcgacaacctgctgcaggagatgctgctgggcggctccccctccgacgccccccacgcccaccaccccctgcacccccacctgatgcaggagcacatgggcaccaacgtgatcgtggccaacaccatgcccacccacctgtccaacggccagatgtgcgagtggccccgcccccgcggccaggccgccacccccgagaccccccagccctccccccccggcggctccggctccgagccctacaagctgctgcccggcgccgtggccaccatcgtgaagcccctgtccgccatcccccagcccaccatcaccaagcaggaggtgatctaa

SEQ ID NO: 3 - protein, 22 aa, synthetic construct
MDMRVPAQLLGLLLLWLSGARC

SEQ ID NO: 4 - protein, 19 aa, synthetic construct
MEFGLSWVFLVALFRGVQC

SEQ ID NO: 5 - protein, 20 aa, synthetic construct
METDTLLLWVLLLWVPGSTG

SEQ ID NO: 6 - protein, 19 aa, synthetic construct
MKWVTFISLLFLFSSSSRA

SEQ ID NO: 7 - protein, 19 aa, synthetic construct
MRMQLLLLIALSLALVTNS

SEQ ID NO: 8 - RNA, 129 nt, synthetic construct
  source 1..129: mol_type = other RNA
tctcaacacaacatatacaaaacaaacgaatctcaagcaatcaagcattctacttctattgcagcaatttaaatcatttcttttaaagcaaaagcaattttctgaaaattttcaccatttacgaacgat

SEQ ID NO: 9 - RNA, 49 nt, synthetic construct
  source 1..49: mol_type = other RNA
cgaactagtattcttctggtccccacagactcagagagaacccgccacc

SEQ ID NO: 10 - RNA, 15 nt, synthetic construct
  source 1..15: mol_type = other RNA
aggagggtttttacc

SEQ ID NO: 11 - RNA, 122 nt, synthetic construct
  source 1..122: mol_type = other RNA
aaccagcctcaagaacacccgaatggagtctctaagctacataataccaacttacacttacaaaatgttgtcccccaaaatgtagccattcgtatctgctcctaataaaaagaaagtttctt

SEQ ID NO: 12 - RNA, 159 nt, synthetic construct
  source 1..159: mol_type = other RNA
agtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattct

SEQ ID NO: 13 - RNA, 113 nt, synthetic construct
  source 1..113: mol_type = other RNA
taagctggagcctcggtggccatgcttcttgccccttgggcctccccccagcccctcctccccttcctgcacccgtacccccgtggtctttgaataaagtctgagtgggcggc

SEQ ID NO: 14 - RNA, 325 nt, synthetic construct
  source 1..325: mol_type = other RNA
ttctagaggatccagatcttaagtaagtaagctcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtcgagagct

SEQ ID NO: 15 - RNA, 136 nt, synthetic construct
  source 1..136: mol_type = other RNA
tcccagaggttcacaatgttagtgatgggcgctgaaagatgcgtagctacccttctggagccacttcctggtggtaagcagaaatccaagggtacggtggtacggtggaaagcagtcccagctctgcattgggaac

SEQ ID NO: 16 - RNA, 142 nt, synthetic construct
  source 1..142: mol_type = other RNA
aacaacaactgctaacgagctcgtaagcccacattccatcctttccatttctgagatggttcttaatgatccattccctggcaaacttctctgagctttatagctttgtaatgcatgccttggctctaatgggtttcatctt

SEQ ID NO: 17 - RNA, 325 nt, synthetic construct; identical to SEQ ID NO: 14
  source 1..325: mol_type = other RNA

SEQ ID NO: 18 - DNA, 1839 bp, synthetic construct
  source 1..1839: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgataacgccaccatgcgcctgtccaagaccctggtggacatggacatggccgactactccgccgccctggaccccgcctacaccaccctggagttcgagaacgtgcaggtgctgaccatgggcaacgacacctccccctccgagggcaccaacctgaacgcccccaactccctgggcgtgtccgccctgtgcgccatctgcggcgaccgcgccaccggcaagcactacggcgcctcctcctgcgacggctgcaagggcttcttccgccgctccgtgcgcaagaaccacatgtactcctgccgcttctcccgccagtgcgtggtggacaaggacaagcgcaaccagtgccgctactgccgcctgaagaagtgcttccgcgccggcatgaagaaggaggccgtgcagaacgagcgcgaccgcatctccacccgccgctcctcctacgaggactcctccctgccctccatcaacgccctgctgcaggccgaggtgctgtcccgccagatcacctcccccgtgtccggcatcaacggcgacatccgcgccaagaagatcgcctccatcgccgacgtgtgcgagtccatgaaggagcagctgctggtgctggtggagtgggccaagtacatccccgccttctgcgagctgcccctggacgaccaggtggccctgctgcgcgcccacgccggcgagcacctgctgctgggcgccaccaagcgctccatggtgttcaaggacgtgctgctgctgggcaacgactacatcgtgccccgccactgccccgagctggccgagatgtcccgcgtgtccatccgcatcctggacgagctggtgctgcccttccaggagctgcagatcgacgacaacgagtacgcctacctgaaggccatcatcttcttcgaccccgacgccaagggcctgtccgaccccggcaagatcaagcgcctgcgctcccaggtgcaggtgtccctggaggactacatcaacgaccgccagtacgactcccgcggccgcttcggcgagctgctgctgctgctgcccaccctgcagtccatcacctggcagatgatcgagcagatccagttcatcaagctgttcggcatggccaagatcgacaacctgctgcaggagatgctgctgggcggctccccctccgacgccccccacgcccaccaccccctgcacccccacctgatgcaggagcacatgggcaccaacgtgatcgtggccaacaccatgcccacccacctgtccaacggccagatgtgcgagtggccccgcccccgcggccaggccgccacccccgagaccccccagccctccccccccggcggctccggctccgagccctacaagctgctgcccggcgccgtggccaccatcgtgaagcccctgtccgccatcccccagcccaccatcaccaagcaggaggtgatctaaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - DNA, 1425 bp, synthetic construct
  source 1..1425: mol_type = other DNA
atgcgcctgtccaagaccctggtggacatggacatggccgactactccgccgccctggaccccgcctacaccaccctggagttcgagaacgtgcaggtgctgaccatgggcaacgacacctccccctccgagggcaccaacctgaacgcccccaactccctgggcgtgtccgccctgtgcgccatctgcggcgaccgcgccaccggcaagcactacggcgcctcctcctgcgacggctgcaagggcttcttccgccgctccgtgcgcaagaaccacatgtactcctgccgcttctcccgccagtgcgtggtggacaaggacaagcgcaaccagtgccgctactgccgcctgaagaagtgcttccgcgccggcatgaagaaggaggccgtgcagaacgagcgcgaccgcatctccacccgccgctcctcctacgaggactcctccctgccctccatcaacgccctgctgcaggccgaggtgctgtcccgccagatcacctcccccgtgtccggcatcaacggcgacatccgcgccaagaagatcgcctccatcgccgacgtgtgcgagtccatgaaggagcagctgctggtgctggtggagtgggccaagtacatccccgccttctgcgagctgcccctggacgaccaggtggccctgctgcgcgcccacgccggcgagcacctgctgctgggcgccaccaagcgctccatggtgttcaaggacgtgctgctgctgggcaacgactacatcgtgccccgccactgccccgagctggccgagatgtcccgcgtgtccatccgcatcctggacgagctggtgctgcccttccaggagctgcagatcgacgacaacgagtacgcctacctgaaggccatcatcttcttcgaccccgacgccaagggcctgtccgaccccggcaagatcaagcgcctgcgctcccaggtgcaggtgtccctggaggactacatcaacgaccgccagtacgactcccgcggccgcttcggcgagctgctgctgctgctgcccaccctgcagtccatcacctggcagatgatcgagcagatccagttcatcaagctgttcggcatggccaagatcgacaacctgctgcaggagatgctgctgggcggctccccctccgacgccccccacgcccaccaccccctgcacccccacctgatgcaggagcacatgggcaccaacgtgatcgtggccaacaccatgcccacccacctgtccaacggccagatgtgcgagtggccccgcccccgcggccaggccgccacccccgagaccccccagccctccccccccggcggctccggctccgagccctacaagctgctgcccggcgccgtggccaccatcgtgaagcccctgtccgccatcccccagcccaccatcaccaagcaggaggtgatctaa

SEQ ID NO: 20 - DNA, 1836 bp, synthetic construct
  source 1..1836: mol_type = other DNA
agctcattaatacgactcactatacaaaacaaacgaatctcaagcaatcaagcattctacttctattgcagcaatttaaatcatttcttttaaagcaaaagcaattttctgaaaattttcaccatttacgaacgataacgccaccatgcgcctgtccaagaccctggtggacatggacatggccgactactccgccgccctggaccccgcctacaccaccctggagttcgagaacgtgcaggtgctgaccatgggcaacgacacctccccctccgagggcaccaacctgaacgcccccaactccctgggcgtgtccgccctgtgcgccatctgcggcgaccgcgccaccggcaagcactacggcgcctcctcctgcgacggctgcaagggcttcttccgccgctccgtgcgcaagaaccacatgtactcctgccgcttctcccgccagtgcgtggtggacaaggacaagcgcaaccagtgccgctactgccgcctgaagaagtgcttccgcgccggcatgaagaaggaggccgtgcagaacgagcgcgaccgcatctccacccgccgctcctcctacgaggactcctccctgccctccatcaacgccctgctgcaggccgaggtgctgtcccgccagatcacctcccccgtgtccggcatcaacggcgacatccgcgccaagaagatcgcctccatcgccgacgtgtgcgagtccatgaaggagcagctgctggtgctggtggagtgggccaagtacatccccgccttctgcgagctgcccctggacgaccaggtggccctgctgcgcgcccacgccggcgagcacctgctgctgggcgccaccaagcgctccatggtgttcaaggacgtgctgctgctgggcaacgactacatcgtgccccgccactgccccgagctggccgagatgtcccgcgtgtccatccgcatcctggacgagctggtgctgcccttccaggagctgcagatcgacgacaacgagtacgcctacctgaaggccatcatcttcttcgaccccgacgccaagggcctgtccgaccccggcaagatcaagcgcctgcgctcccaggtgcaggtgtccctggaggactacatcaacgaccgccagtacgactcccgcggccgcttcggcgagctgctgctgctgctgcccaccctgcagtccatcacctggcagatgatcgagcagatccagttcatcaagctgttcggcatggccaagatcgacaacctgctgcaggagatgctgctgggcggctccccctccgacgccccccacgcccaccaccccctgcacccccacctgatgcaggagcacatgggcaccaacgtgatcgtggccaacaccatgcccacccacctgtccaacggccagatgtgcgagtggccccgcccccgcggccaggccgccacccccgagaccccccagccctccccccccggcggctccggctccgagccctacaagctgctgcccggcgccgtggccaccatcgtgaagcccctgtccgccatcccccagcccaccatcaccaagcaggaggtgatctaaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 21 - protein, 465 aa, synthetic construct
MRLSKTLLVDMDMADYSAALDPAYTTLEFENVQVLTMGNDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMSTPETPQPSPPGGSGSEPYKLLPGAVATIVKPLSAIPQPTITKQEVI

SEQ ID NO: 22 - RNA, 1395 nt, synthetic construct
  source 1..1395: mol_type = other RNA
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 23 - DNA, 1812 bp, synthetic construct
  source 1..1812: mol_type = other DNA
agctcattaatacgactcactatacaaaacaaacgaatctcaagcaatcaagcattctacttctattgcagcaatttaaatcatttcttttaaagcaaaagcaattttctgaaaattttcaccatttacgaacgatagcgctgccaccatgcgcctgtccaagaccctggtggacatggacatggccgactactccgccgccctggaccccgcctacaccaccctggagttcgagaacgtgcaggtgctgaccatgggcaacgacacctccccctccgagggcaccaacctgaacgcccccaactccctgggcgtgtccgccctgtgcgccatctgcggcgaccgcgccaccggcaagcactacggcgcctcctcctgcgacggctgcaagggcttcttccgccgctccgtgcgcaagaaccacatgtactcctgccgcttctcccgccagtgcgtggtggacaaggacaagcgcaaccagtgccgctactgccgcctgaagaagtgcttccgcgccggcatgaagaaggaggccgtgcagaacgagcgcgaccgcatctccacccgccgctcctcctacgaggactcctccctgccctccatcaacgccctgctgcaggccgaggtgctgtcccgccagatcacctcccccgtgtccggcatcaacggcgacatccgcgccaagaagatcgcctccatcgccgacgtgtgcgagtccatgaaggagcagctgctggtgctggtggagtgggccaagtacatccccgccttctgcgagctgcccctggacgaccaggtggccctgctgcgcgcccacgccggcgagcacctgctgctgggcgccaccaagcgctccatggtgttcaaggacgtgctgctgctgggcaacgactacatcgtgccccgccactgccccgagctggccgagatgtcccgcgtgtccatccgcatcctggacgagctggtgctgcccttccaggagctgcagatcgacgacaacgagtacgcctacctgaaggccatcatcttcttcgaccccgacgccaagggcctgtccgaccccggcaagatcaagcgcctgcgctcccaggtgcaggtgtccctggaggactacatcaacgaccgccagtacgactcccgcggccgcttcggcgagctgctgctgctgctgcccaccctgcagtccatcacctggcagatgatcgagcagatccagttcatcaagctgttcggcatggccaagatcgacaacctgctgcaggagatgctgctgggcggctccccctccgacgccccccacgcccaccaccccctgcacccccacctgatgcaggagcacatgggcaccaacgtgatcgtggccaacaccatgcccacccacctgtccaacggccagatgtccacccccgagaccccccagccctccccccccggcggctccggctccgagccctacaagctgctgcccggcgccgtggccaccatcgtgaagcccctgtccgccatcccccagcccaccatcaccaagcaggaggtgatctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 24 - RNA, 112 nt, synthetic construct
  source 1..112: mol_type = other RNA
caaaacaaacgaatctcaagcaatcaagcattctacttctattgcagcaatttaaatcatttcttttaaagcaaaagcaattttctgaaaattttcaccatttacgaacgat

SEQ ID NO: 25 - RNA, 122 nt, synthetic construct; identical to SEQ ID NO: 11
  source 1..122: mol_type = other RNA

SEQ ID NO: 26 - DNA, 1395 bp, synthetic construct
  source 1..1395: mol_type = other DNA
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 27 - RNA, 1839 nt, synthetic construct
  source 1..1839: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgataacgccaccatgcgcctgtccaagaccctggtggacatggacatggccgactactccgccgccctggaccccgcctacaccaccctggagttcgagaacgtgcaggtgctgaccatgggcaacgacacctccccctccgagggcaccaacctgaacgcccccaactccctgggcgtgtccgccctgtgcgccatctgcggcgaccgcgccaccggcaagcactacggcgcctcctcctgcgacggctgcaagggcttcttccgccgctccgtgcgcaagaaccacatgtactcctgccgcttctcccgccagtgcgtggtggacaaggacaagcgcaaccagtgccgctactgccgcctgaagaagtgcttccgcgccggcatgaagaaggaggccgtgcagaacgagcgcgaccgcatctccacccgccgctcctcctacgaggactcctccctgccctccatcaacgccctgctgcaggccgaggtgctgtcccgccagatcacctcccccgtgtccggcatcaacggcgacatccgcgccaagaagatcgcctccatcgccgacgtgtgcgagtccatgaaggagcagctgctggtgctggtggagtgggccaagtacatccccgccttctgcgagctgcccctggacgaccaggtggccctgctgcgcgcccacgccggcgagcacctgctgctgggcgccaccaagcgctccatggtgttcaaggacgtgctgctgctgggcaacgactacatcgtgccccgccactgccccgagctggccgagatgtcccgcgtgtccatccgcatcctggacgagctggtgctgcccttccaggagctgcagatcgacgacaacgagtacgcctacctgaaggccatcatcttcttcgaccccgacgccaagggcctgtccgaccccggcaagatcaagcgcctgcgctcccaggtgcaggtgtccctggaggactacatcaacgaccgccagtacgactcccgcggccgcttcggcgagctgctgctgctgctgcccaccctgcagtccatcacctggcagatgatcgagcagatccagttcatcaagctgttcggcatggccaagatcgacaacctgctgcaggagatgctgctgggcggctccccctccgacgccccccacgcccaccaccccctgcacccccacctgatgcaggagcacatgggcaccaacgtgatcgtggccaacaccatgcccacccacctgtccaacggccagatgtgcgagtggccccgcccccgcggccaggccgccacccccgagaccccccagccctccccccccggcggctccggctccgagccctacaagctgctgcccggcgccgtggccaccatcgtgaagcccctgtccgccatcccccagcccaccatcaccaagcaggaggtgatctaaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - DNA, 1425 bp, Homo sapiens
  source 1..1425: mol_type = genomic DNA
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 29 - RNA, 1836 nt, synthetic construct
  source 1..1836: mol_type = other RNA
agctcattaatacgactcactatacaaaacaaacgaatctcaagcaatcaagcattctacttctattgcagcaatttaaatcatttcttttaaagcaaaagcaattttctgaaaattttcaccatttacgaacgataacgccaccatgcgcctgtccaagaccctggtggacatggacatggccgactactccgccgccctggaccccgcctacaccaccctggagttcgagaacgtgcaggtgctgaccatgggcaacgacacctccccctccgagggcaccaacctgaacgcccccaactccctgggcgtgtccgccctgtgcgccatctgcggcgaccgcgccaccggcaagcactacggcgcctcctcctgcgacggctgcaagggcttcttccgccgctccgtgcgcaagaaccacatgtactcctgccgcttctcccgccagtgcgtggtggacaaggacaagcgcaaccagtgccgctactgccgcctgaagaagtgcttccgcgccggcatgaagaaggaggccgtgcagaacgagcgcgaccgcatctccacccgccgctcctcctacgaggactcctccctgccctccatcaacgccctgctgcaggccgaggtgctgtcccgccagatcacctcccccgtgtccggcatcaacggcgacatccgcgccaagaagatcgcctccatcgccgacgtgtgcgagtccatgaaggagcagctgctggtgctggtggagtgggccaagtacatccccgccttctgcgagctgcccctggacgaccaggtggccctgctgcgcgcccacgccggcgagcacctgctgctgggcgccaccaagcgctccatggtgttcaaggacgtgctgctgctgggcaacgactacatcgtgccccgccactgccccgagctggccgagatgtcccgcgtgtccatccgcatcctggacgagctggtgctgcccttccaggagctgcagatcgacgacaacgagtacgcctacctgaaggccatcatcttcttcgaccccgacgccaagggcctgtccgaccccggcaagatcaagcgcctgcgctcccaggtgcaggtgtccctggaggactacatcaacgaccgccagtacgactcccgcggccgcttcggcgagctgctgctgctgctgcccaccctgcagtccatcacctggcagatgatcgagcagatccagttcatcaagctgttcggcatggccaagatcgacaacctgctgcaggagatgctgctgggcggctccccctccgacgccccccacgcccaccaccccctgcacccccacctgatgcaggagcacatgggcaccaacgtgatcgtggccaacaccatgcccacccacctgtccaacggccagatgtgcgagtggccccgcccccgcggccaggccgccacccccgagaccccccagccctccccccccggcggctccggctccgagccctacaagctgctgcccggcgccgtggccaccatcgtgaagcccctgtccgccatcccccagcccaccatcaccaagcaggaggtgatctaaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - RNA, 1392 nt, synthetic construct
  source 1..1392: mol_type = other RNA
atgcgcctgtccaagaccctggtggacatggacatggccgactactccgccgccctggaccccgcctacaccaccctggagttcgagaacgtgcaggtgctgaccatgggcaacgacacctccccctccgagggcaccaacctgaacgcccccaactccctgggcgtgtccgccctgtgcgccatctgcggcgaccgcgccaccggcaagcactacggcgcctcctcctgcgacggctgcaagggcttcttccgccgctccgtgcgcaagaaccacatgtactcctgccgcttctcccgccagtgcgtggtggacaaggacaagcgcaaccagtgccgctactgccgcctgaagaagtgcttccgcgccggcatgaagaaggaggccgtgcagaacgagcgcgaccgcatctccacccgccgctcctcctacgaggactcctccctgccctccatcaacgccctgctgcaggccgaggtgctgtcccgccagatcacctcccccgtgtccggcatcaacggcgacatccgcgccaagaagatcgcctccatcgccgacgtgtgcgagtccatgaaggagcagctgctggtgctggtggagtgggccaagtacatccccgccttctgcgagctgcccctggacgaccaggtggccctgctgcgcgcccacgccggcgagcacctgctgctgggcgccaccaagcgctccatggtgttcaaggacgtgctgctgctgggcaacgactacatcgtgccccgccactgccccgagctggccgagatgtcccgcgtgtccatccgcatcctggacgagctggtgctgcccttccaggagctgcagatcgacgacaacgagtacgcctacctgaaggccatcatcttcttcgaccccgacgccaagggcctgtccgaccccggcaagatcaagcgcctgcgctcccaggtgcaggtgtccctggaggactacatcaacgaccgccagtacgactcccgcggccgcttcggcgagctgctgctgctgctgcccaccctgcagtccatcacctggcagatgatcgagcagatccagttcatcaagctgttcggcatggccaagatcgacaacctgctgcaggagatgctgctgggcggctccccctccgacgccccccacgcccaccaccccctgcacccccacctgatgcaggagcacatgggcaccaacgtgatcgtggccaacaccatgcccacccacctgtccaacggccagatgtccacccccgagaccccccagccctccccccccggcggctccggctccgagccctacaagctgctgcccggcgccgtggccaccatcgtgaagcccctgtccgccatcccccagcccaccatcaccaagcaggaggtgatc

SEQ ID NO: 31 - RNA, 10 nt, synthetic construct
  source 1..10: mol_type = other RNA
gccaccatgc

SEQ ID NO: 32 - RNA, 1812 nt, synthetic construct
  source 1..1812: mol_type = other RNA
agctcattaatacgactcactatacaaaacaaacgaatctcaagcaatcaagcattctacttctattgcagcaatttaaatcatttcttttaaagcaaaagcaattttctgaaaattttcaccatttacgaacgatagcgctgccaccatgcgcctgtccaagaccctggtggacatggacatggccgactactccgccgccctggaccccgcctacaccaccctggagttcgagaacgtgcaggtgctgaccatgggcaacgacacctccccctccgagggcaccaacctgaacgcccccaactccctgggcgtgtccgccctgtgcgccatctgcggcgaccgcgccaccggcaagcactacggcgcctcctcctgcgacggctgcaagggcttcttccgccgctccgtgcgcaagaaccacatgtactcctgccgcttctcccgccagtgcgtggtggacaaggacaagcgcaaccagtgccgctactgccgcctgaagaagtgcttccgcgccggcatgaagaaggaggccgtgcagaacgagcgcgaccgcatctccacccgccgctcctcctacgaggactcctccctgccctccatcaacgccctgctgcaggccgaggtgctgtcccgccagatcacctcccccgtgtccggcatcaacggcgacatccgcgccaagaagatcgcctccatcgccgacgtgtgcgagtccatgaaggagcagctgctggtgctggtggagtgggccaagtacatccccgccttctgcgagctgcccctggacgaccaggtggccctgctgcgcgcccacgccggcgagcacctgctgctgggcgccaccaagcgctccatggtgttcaaggacgtgctgctgctgggcaacgactacatcgtgccccgccactgccccgagctggccgagatgtcccgcgtgtccatccgcatcctggacgagctggtgctgcccttccaggagctgcagatcgacgacaacgagtacgcctacctgaaggccatcatcttcttcgaccccgacgccaagggcctgtccgaccccggcaagatcaagcgcctgcgctcccaggtgcaggtgtccctggaggactacatcaacgaccgccagtacgactcccgcggccgcttcggcgagctgctgctgctgctgcccaccctgcagtccatcacctggcagatgatcgagcagatccagttcatcaagctgttcggcatggccaagatcgacaacctgctgcaggagatgctgctgggcggctccccctccgacgccccccacgcccaccaccccctgcacccccacctgatgcaggagcacatgggcaccaacgtgatcgtggccaacaccatgcccacccacctgtccaacggccagatgtccacccccgagaccccccagccctccccccccggcggctccggctccgagccctacaagctgctgcccggcgccgtggccaccatcgtgaagcccctgtccgccatcccccagcccaccatcaccaagcaggaggtgatctaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 33 - DNA, 1392 bp, Synthetic construct
  source 1..1392: mol_type = other DNA
atgcgcctgtccaagaccctggtggacatggacatggccgactactccgccgccctggaccccgcctacaccaccctggagttcgagaacgtgcaggtgctgaccatgggcaacgacacctccccctccgagggcaccaacctgaacgcccccaactccctgggcgtgtccgccctgtgcgccatctgcggcgaccgcgccaccggcaagcactacggcgcctcctcctgcgacggctgcaagggcttcttccgccgctccgtgcgcaagaaccacatgtactcctgccgcttctcccgccagtgcgtggtggacaaggacaagcgcaaccagtgccgctactgccgcctgaagaagtgcttccgcgccggcatgaagaaggaggccgtgcagaacgagcgcgaccgcatctccacccgccgctcctcctacgaggactcctccctgccctccatcaacgccctgctgcaggccgaggtgctgtcccgccagatcacctcccccgtgtccggcatcaacggcgacatccgcgccaagaagatcgcctccatcgccgacgtgtgcgagtccatgaaggagcagctgctggtgctggtggagtgggccaagtacatccccgccttctgcgagctgcccctggacgaccaggtggccctgctgcgcgcccacgccggcgagcacctgctgctgggcgccaccaagcgctccatggtgttcaaggacgtgctgctgctgggcaacgactacatcgtgccccgccactgccccgagctggccgagatgtcccgcgtgtccatccgcatcctggacgagctggtgctgcccttccaggagctgcagatcgacgacaacgagtacgcctacctgaaggccatcatcttcttcgaccccgacgccaagggcctgtccgaccccggcaagatcaagcgcctgcgctcccaggtgcaggtgtccctggaggactacatcaacgaccgccagtacgactcccgcggccgcttcggcgagctgctgctgctgctgcccaccctgcagtccatcacctggcagatgatcgagcagatccagttcatcaagctgttcggcatggccaagatcgacaacctgctgcaggagatgctgctgggcggctccccctccgacgccccccacgcccaccaccccctgcacccccacctgatgcaggagcacatgggcaccaacgtgatcgtggccaacaccatgcccacccacctgtccaacggccagatgtccacccccgagaccccccagccctccccccccggcggctccggctccgagccctacaagctgctgcccggcgccgtggccaccatcgtgaagcccctgtccgccatcccccagcccaccatcaccaagcaggaggtgatc